(19) United States
(12) Patent Application Publication
(10) Pub. No.: US 2011/0306579 A1
(43) Pub. Date: Dec. 15, 2011

(54) METHODS OF NEUROPROTECTION USING NEUROPROTECTIVE STEROIDS AND A VITAMIN D

(75) Inventor: Donald G. Stein, Atlanta, GA (US)

(73) Assignee: EMORY UNIVERSITY, Atlanta, GA (US)

(21) Appl. No.: 13/146,585

(22) PCT Filed: Jan. 28, 2010
(86) PCT No.: PCT/US10/22433

§ 371 Date: Jul. 27, 2011

Related U.S. Application Data
(60) Provisional application No. 61/148,814, filed on Jan. 30, 2009.

(51) Int. Cl. A61K 31/59; A61K 31/593; A61P 25/00; A61K 31/592

(52) U.S. Cl. 514/167

(57) ABSTRACT

Described herein are compositions and methods for treating or preventing nervous system injury. In particular, the methods and compositions relate to the use of at least one neuroprotective steroid, such as progesterone, and vitamin D.

### RELATED APPLICATIONS

This application claims the priority benefits under 35 U.S.C. §119(e) to U.S. provisional application 61/148,814, filed Jan. 30, 2009, the entire contents of which are incorporated herein by reference.

### STATEMENT OF GOVERNMENT SUPPORT

This invention was made using U.S. government funds under NIH grants #1RO1N540825 and #1RO1N538664 and the government has certain rights in the invention.

### FIELD OF THE INVENTION

The present invention is in the area of pharmaceutical chemistry and specifically relates to diagnostic methods and uses of vitamin D and analogs in combination or alternation with certain neuroprotective steroids in treatment of nervous system injury or nervous system inflammation related to injury or disease. Certain pharmaceutical compositions are also provided that allow enhanced recovery of neurological functions after inflammation or injury that include vitamin D in combination with certain steroid compounds, in particular progesterone or its active metabolites, prodrugs, and analogs.

### BACKGROUND OF THE INVENTION

Brain injuries, including traumatic brain injury (TBI) and stroke, affect well over 2 million Americans each year and are a significant health concern worldwide. There are currently approximately 5.7 million stroke survivors in the US, many with permanent disabilities, and more than 5 million Americans who have suffered a TBI resulting in the permanent need for help in performing daily activities. Traumatic brain injuries result from a blow or jolt to the head or a penetrating head injury that disrupts the function of the brain, with severity ranging from “mild,” i.e., a brief change in mental status or consciousness to “severe,” i.e., an extended period of unconsciousness or amnesia after the injury. In contrast, strokes are a result of diseases that affect the blood vessels that supply blood to the brain. A stroke occurs when a blood vessel that brings oxygen and nutrients to the brain either bursts (hemorrhagic stroke) or is clogged by a blood clot or some other mass (ischemic stroke). The majority of strokes are ischemic, however hemorrhagic strokes typically result in more severe injuries.

Despite several decades of effort, scientists have not yet found a pharmacological agent that consistently improves outcomes after brain injuries such as stroke or TBI (see Sauerland, S. et al., Lancet 2004, 364, 1291-1292; Brain Trauma Foundation, American Association of Neurological Surgeons, Joint Section on Neurotrauma and Critical Care. Guidelines for the management of severe head injury. J. Neurotrauma 1996, 13, 641-734).

After TBI or stroke, inflammation is a primary cause of secondary damage and long-term damage. Following insults to the central nervous system, a cascade of physiological events leads to neuronal loss including, for example, an inflammatory immune response and excitotoxicity resulting from disrupting the glutamate, acetylcholine, cholinergic, GABA_{A}, and NMDA receptor systems. In these cases, a complex cascade of events leads to the delivery of blood-borne leucocytes to sites of injury to kill potential pathogens and promote tissue repair. However, the powerful inflammatory response has the capacity to cause damage to normal tissue, and dysregulation of the innate, or acquired immune response is involved in different pathologies.

In addition to TBI and stroke, inflammation is being recognized as a key component of a variety of nervous system disorders. It has long been known that certain diseases such as multiple sclerosis are due to inflammation in the central nervous system, but it is only in recent years that it has been suggested that inflammation may significantly contribute to neurodegenerative disorders such as HIV-related dementia, Alzheimer's and prion diseases. It is now known that the resident macrophages of the central nervous system (CNS), the microglia, when activated may secrete molecules that cause neuronal dysfunction, or degeneration.

While TBI is a leading cause of death and disability among people of all ages in the United States, the rate of death from TBI has declined for most age groups over the past ten years due in large part to improved safety measures such as the use of safety belts. However, the rate of TBI in the elderly it has risen by over 21% (Langlois et al., 2004) and is currently more than twice that of the younger population (Mosenthal et al., 2002). In addition, the risk of stroke increases with age. For each decade after age 55, the risk of stroke doubles and in each year, more than 70 percent of people who suffer a stroke are over the age of 65.

In addition to higher incidence of neurological injuries and disorders, the elderly are also often subject to alterations in certain systemic hormonal levels that may significantly affect their response to injury (Topinkova, 2008). Aside from advanced age, itself a major predictor of injury severity, other potentially exacerbating factors in the aged include systemic issues such as kidney disease, hypertension, atherosclerosis and cardiovascular disease diabetes, cancer and hormonal imbalances such as hyperparathyroidism. While all of these conditions can affect responscs to injury, cach has also been associated in the growing literature with insufficient serum levels of vitamin D as often ignored underlying problem (Grant, 2006; Holick and Chen, 2008; Peterlik and Cross, 2005).

Vitamin D is the term used for a group of fat-soluble prohormones, the two major forms of which are vitamin D₂ (or ergocalciferol) and vitamin D₃ (or cholecalciferol). The term “vitamin D” also refers to metabolites and other analogues of these substances. Vitamin D has historically been known to play an important role in the maintenance of organ systems. For example, vitamin D enables normal mineralization of bone and prevents hypocalccmic tetany and is needed for bone growth and bone remodeling by osteoblasts and osteoclasts, inhibits parathyroid hormone secretion from the parathyroid gland and affects the immune system by promoting phagocytosis, anti-tumor activity, and immunomodulatory functions.

Vitamin D deficiency (D-deficiency) is associated with rickets in children and osteomalacia in adults, but has recently also been linked to other systemic conditions such as secondary hyperparathyroidism (Holick, 2005a; McCarty, 2005), metabolic syndrome (Peterlik and Cross, 2005), hypertension (Li et al., 2002; Wang et al., 2008), obesity (Rajakumar et al., 2008), and diabetes mellitus (Giulietti et al., 2004; Grant, 2006), and cardiovascular disease outcomes such as stroke and congestive heart failure (Michos and Melamed, 2008; Vieth and Kimball, 2006).

Several recent studies also suggest that inadequate vitamin D may predispose towards Parkinson's and other neurodegenerative diseases, mood disorders (Garcion et al., 2002; Kalueff et al., 2004a), and even tuberculosis infection (Zasloff, 2006). Vitamin D deficiency has been associated with increased incidence of multiple sclerosis (MS), Sjögren's syndrome, rheumatoid arthritis, and Crohn's disease. Systemic vitamin D levels have been suggested as a possible explanation for the latitudinal gradient in MS incidence (nearly zero at the equator and increasing with greater distance from it), and correlations have been observed between circulating vitamin D status and the risk of developing MS, as well as a protective effect of vitamin D intake in both human disease and animal models. Vitamin D therapy for MS has been shown to be safe in humans and has recently been recommended for use in double blind controlled clinical trials. Vitamin D deficiencies have also been linked to increased risks of stroke, particularly fatal stroke (Pilz, et al. (2008) Stroke 39:2611-3; Poole (2006) Stroke 37:243).

With respect to inflammation, vitamin D has been shown to decrease levels of pro-inflammatory T_{H}1 cytokines TNFα, IL-1β, IL-12, IL-6, IFNγ; the downstream reactive oxygen species generated by activated macrophages and NF-κB, the central mediator of inflammation which has also been linked with stress-response in humans and stress-induced neuronal loss in rats. Long-term vitamin D deficiency hass been shown to lead to generalized inflammatory conditions that compromise the cardiovascular system and glucose metabolism. In acute injury, chronic D-deficiency leads to a more intense pro-inflammatory type 1 reaction.

A low level of vitamin D is a key marker of frailty, defined as a “global impairment of physiological reserves involving multiple organ systems”. Frailty often results in a reduced capacity to maintain physical and psychosocial homeostasis and greater vulnerability to internal and environmental stressors such as trauma. This could be especially important in the elderly, who are already more vulnerable to TBI, and studies have shown that advanced age is a major predictor of injury severity after TBI. Other potentially exacerbating factors in the aged include systemic issues such as kidney disease, hypertension, atherosclerosis and cardiovascular disease, diabetes, cancer, and hormonal imbalances such as hyperparathyroidism. While all these conditions can independently affect responses to injury, each has also been associated by a growing literature with insufficient serum levels of vitamin D as a key and often ignored underlying problem. Vitamin D status has been specifically associated with functional outcomes in the elderly, suggesting that supplementation could be especially helpful for this segment of the population.

Vitamin D deficiency can result from inadequate intake coupled with inadequate sunlight exposure, disorders that limit its absorption and conditions that impair conversion of vitamin D into active metabolites such as liver or kidney disorders or a number of hereditary disorders. Vitamin D deficiency is very common in industrialized countries and affects certain subsets of the population particularly the old, the ill, and the institutionalized (Calvo and Whiting 2003).

Vitamin D and its metabolites are largely bound in the blood by vitamin D binding protein (DBP), also known as group-specific component of serum or Gc-globulin. DBP serves as the main reservoir and transporter of the vitamin 13 endocrine system, and binds about 88% of the total 25OHD3 and 85% of the total VDH in serum. Only about 5% of DBP is bound to vitamin D metabolites, and its serum concentration is about 20-fold that of the various vitamin D species. DBP is an acute phase protein produced by the liver, and is upregulated by estrogen and during pregnancy when progesterone (PROG) is also very elevated.

There is growing experimental evidence that progesterone, its metabolites and other gonadal steroids such as estrogen and possibly testosterone, are effective neuroprotective agents. Pre-clinical and clinical research demonstrates that the hormone progesterone is a potent neurosteroid that, acutely administered, can dramatically reduce cerebral edema, inflammation, tissue necrosis, and programmed cell death (see Djebaili, M. et al, J. Neurotrauma 2005, 22, 106-118; Pettus, E. H. et al., Brain Res. 2005, 1049, 112-119; Grossman, K. J. et al., Brain Res, 2004, 1008, 29-39; He, J. et al., Exp. Neurol. 2004, 189, 404-412; He, J. et al., Restor. Neurol. Neurosci. 2004, 22, 19-31; Djebaili, M. et al., J. Neuroscience 2004, 123, 349-359; Hoffman, S. W. et al., Academy of Emergency Medicine, 2001, 8, 496-497; and Wright, D. W. et al., J. Neurotrauma. 2001, 18, 901-909).

In vivo data has demonstrated progesterone's neuroprotective effects in injured nervous systems. For example, following a contusion injury, progesterone reduces the severity of post injury cerebral edema. The attenuation of edema by progesterone is accompanied by the sparing of neurons from secondary neuronal death and improvements in cognitive outcome (Roof et al. (1994) Experimental Neurology 129:64-69). Furthermore, following ischemic injury in rats, progesterone has been shown to reduce cell damage and neurological deficit (Jiang et al. (1996) Brain Research 735:101-107). A Phase II, single-center, controlled trial involving 100 moderate to severe TBI patients showed that 3 days of intravenous progesterone treatment reduced mortality by over 60% and significantly improved functional outcomes at 30 days post-injury (see Wright, D. A. et al., Ann. Emerg. Med. 2007, 49, 391). PCT Publication WO 02/30409 to Emory University provides methods for conferring a neuroprotective effect on a population of cells in a subject following a traumatic injury to the central nervous system by administration of a progestin or progestin metabolite following a traumatic brain injury. PCT Publication WO 06/102644 also to Emory University provides methods for the treatment or the prevention of neuronal damage in the CNS by tapered administration of a progestin or progestin metabolite following a traumatic or ischemic injury to the CNS to avoid withdrawal. In addition, PCT Publication No. WO/2006/102596 to Emory University provides certain methods of treating a subject with a traumatic central nervous system injury, more particularly, a traumatic brain injury that include a therapy comprising a constant or a two-level dosing regime of progesterone.

Although progesterone has been shown to be neuroprotective in traumatic brain injury, its efficacy in stroke is less well defined. However, studies have indicated that progesterone may be useful in treating or preventing neurodegeneration following stroke (see Stein, D. (2005) The Case for Progesterone US Ann. N.Y. Acad. Sci. 1052:152-169; Murphy, et al. (2002) Progesterone Administration During Reperfusion, But Not Prcischemia Alone, Reduces Injury in Ovariectomized Rats. J. Cereb. Blood Flow & Metab. 22:1181-1188; Murphy, et al. (2000) Progesterone Exacerbates Striatal Stroke Injury in Progesterone-Deficient Female Animals. Stroke 31:1173). In addition, U.S. Pat. No. 6,245,757, now expired, to Research Corporation Technologies, Inc. provides a method for the treatment of ischemic damage, such as damage due to stroke or myocardial infarction comprising administering to a mammal afflicted with stroke an effective amount of a neuroprotective steroid in a suitable vehicle.

In addition to being a gonadal steroid, progesterone also belongs to a family of autocrine/paracrine hormones called neurosteroids. Neurosteroids are steroids that accumulate in the brain independently of endocrine sources and which can be synthesized from sterol precursors in nervous cells. These neurosteroids can potentiate GABA transmission, modulate the effects of glutamate, enhance the production of myelin, and prevent release of free radicals from activated microglia.

Various metabolites of progesterone have also been suggested to have neuroprotective properties. For instance, the progesterone metabolites allopregnanolone or epipregnanolone are positive modulators of the GABA receptor, increasing the effects of GABA in a manner that is independent of the benzodiazepines (Baulieu, E. E. (1992) Adv. Biochem. Psychopharmacol. 47:1-16; Robel et al. (1995) Crit. Rev. Neurobiol. 9:383-94; Lambert et al. (1995) Trends Pharmacol. Sci. 16:295-303; Baulieu, E. E. (1997) Recent Prog. Horm. Res. 52:1-32; Reddy et al. (1996) Psychopharmacology 128:280-92). In addition, these neurosteroids act as antagonists at the sigma receptor, which can activate the NMDA channel complex (Maurice et al. (1998) Neuroscience 83:413-28; Maurice et al. (1996) J. Neurosci. Res. 46:734-43; Reddy et al. (1998) Neuroreport 9:3069-73). These neurosteroids have also been shown to reduce the stimulation of cholinergic neurons and the subsequent release of acetylcholine by excitability. Numerous studies have shown that the cholinergic neurons of the basal forebrain are sensitive to injury and that excessive release of acetylcholine can be more excitotoxic than glutamate (Lyeth et al. (1992) J. Neurotrauma 9(2):S463-74; Hayes et al. (1992) J. Neurotrauma 9(1):S173-87).

Although successful in many instances, progesterone treatment may not effectively treat all subsets of patients suffering from neural injury or inflammation. A need remains for improved methods for identifying patients at risk of reduced progesterone response or increased tissue injury, and for improved compositions for enhancing the efficacy of progesterone treatment in patients, in particular in patients characterized as low responders.

### SUMMARY OF THE INVENTION

Provided herein are improved methods of treatment and compositions for treatment of patients suffering from nervous system damage, in particular due to neurodegenerative reactions to injury or disease. In certain instances, the patients are also at risk of suffering from a vitamin D deficiency.

In one embodiment, a pharmaceutical composition is provided that includes a vitamin D in combination with a neuroprotective steroid or a pharmaceutically acceptable salt, ester or prodrug thereof, optionally in a pharmaceutically acceptable carrier. In some embodiments, the vitamin D and the neuroprotective steroid are provided and administered in the same composition; in other embodiments, the vitamin D and the neuroprotective steroid are provided and administered in different compostions, by the same route of administration or by different routes of administration, simultaneously, sequentially, or intermittently.

In particular embodiments, the vitamin D is provided in an amount effective to reverse a vitamin D deficiency in a patient. In specific embodiments, the vitamin D is selected from ergocalciferol, cholecalciferol, calcitriol, seocalcitol, doxercalcifcrol or calcipotrienc. In certain embodiments, the analog is a form of 1,25-dihydroxyvitamin D₃ (1,25-diOH-D), including calcitriol. In specific embodiments, the amount of vitamin D is at least 1000 international units (IU), or at least 1500 IU, or at least 2000 IU, or at least 2500 IU, or at least 3000 IU, or at least 3500 IU, at least 4000 IU, at least 5000 IU, at least 10,000 IU, at least 25,000 IU or at least 50,000 IU or greater.

In some embodiments, the neuroprotective steroid is a progesterone analog or prodrug. In specific embodiments, the neuroprotective steroid is progesterone or allopregnanolone. In some embodiments, the amount of neuroprotective steroid is effective to prevent neurodegeneration at 24 hours after administration, or at 48 hours, or at 72 hours, or at about one week, or at about two weeks, or at about three weeks or at about one month from administration. In certain embodiments, the amount of neuroprotective steroid in a unit dosage is from about 0.1 mg to about 5000 mg, or from about 0.5 mg to about 1000 mg, or from about 1 mg to about 500 mg of the active compound. The composition(s) can be provided for oral or nasal administration, however in other embodiments the composition(s) is/are provided for intravenous or intramuscular administration.

In a separate embodiment, a method of treatment or prevention of a nervous system injury is provided that includes administering a vitamin D in combination or alternation with a neuroprotective steroid or a pharmaceutically acceptable salt, ester or prodrug thereof, optionally in a pharmaceutically acceptable carrier, to a patient suffering from, or at risk of suffering from, such an injury. In certain embodiment, the neuroprotective steroid is a progesterone analog or prodrug. In specific embodiments, the neuroprotective steroid is progesterone or allopregnanolone. In specific embodiments, the vitamin D is selected from ergocalciferol, cholecalciferol, calcitriol, seocalcitol, doxercalciferol or calcipotriene. In certain embodiments, the analog is a form of 1,25-diOH-D, including calcitriol. The nervous system injury can be a traumatic brain injury, but in other embodiments the injury is an ischemic injury such as a stroke. In some embodiments the nervous system injury is a neurodegenerative reaction to injury or disease, traumatic brain injury, ischemic CNS injury, hemorrhagic CNS injury, spinal cord injury, ischemic stroke, hemorrhagic stroke and anterior optic nerve ischemic injury. In certain embodiments, neurodegeneration due to apoptosis is avoided or reduced. The method may enhance physical recovery or reduce loss of function, in particular as related to behavioral or motor function in the patient. In some embodiments, the methods achieve one or more beneficial effects such as (i) reduced neurodegeneration due to apoptosis; (ii) enhanced motor function, (iii) reduced loss of motor function, (iv) reduced inflammation, (v) reduced loss of visual function, and (vi) reduced damage from an inflammatory process.

In spccifid embodiments, the administration of neuroprotective steroid and vitamin D is once a day or less than one day, or less than 18 hours, or less than 12 hours, or less than six hours from the injury. In other embodiments, the administration of neuroprotective steroid and vitamin D is commenced at a time selected from the group consisting of (i) one day from the nervous system injury; (ii) less than one day from the nervous system injury; (iii) less than 18 hours from the nervous system injury; (iv) less than 12 hours from the nervous system injury; and (v) less than six hours from the nervous system injury.

In specific embodiments, the amount of vitamin D provided per administration or per day is at least 1000 international units (IU), or at least 1500 IU, or at least 2000 IU, or at least 2500 IU, or at least 3000 IU, or at least 3500 IU, at least 4000 IU, at least 5000 IU, at least 10,000 IU, at least 25,000 IU or at least 50,000 IU or greater. In specific embodiments, the amount of vitamin D is at least 1000 international units (IU) per day, or at least 1500 IU/day, or at least 2000 IU/day, or at least 2500 IU/day, or at least 3000 IU/day, or at least 3500 IU/day, at least 4000 IU/day, at least 5000 IU/day, at least 10,000 IU/day, at least 25,000 IU/day or at least 50,000 IU/day or greater. In some embodiments, the amount of neuroprotective steroid is effective to prevent neurodcgcncration at 24 hours after administration, or at 48 hours, or at 72 hours, or at about one week, or at about two weeks, or at about three weeks or at about one month from administration. In certain embodiments, the amount of neuroprotective steroid is from about 0.001 mg per kilogram body weight to about 1000 mg/kg, or from about 0.05 mg/kg to about 500 mg/kg, or from about 0.1 mg/kg to about 300 mg/kg. In certain embodiments, the amount of neuroprotective steroid is from about 0.001 mg per kilogram body weight per day to about 1000 mg/kg/day, or from about 0.05 mg/kg/day to about 500 mg/kg/day, or from about 0.1 mg/kg/day to about 300 mg/kg/day. In certain embodiments the administration is via oral or nasal administration, however in other embodiments the administration is via intravenous or intramuscular administration.

In specific embodiments of the invention, methods of treating or preventing damage resulting from inflammatory processes that are initiated by a TBI are provided, comprising administering a vitamin D in combination or alternation with a neuroprotective steroid or a pharmaceutically acceptable salt, ester or prodrug thereof, optionally in a pharmaceutically acceptable carrier to a patient in need thereof, in accordance with any embodiments described above. In certain embodiments, the patient is suffering from a vitamin D deficiency.

In certain embodiments, a method of preventing or reducing inflammatory reactions in a patient is provided that includes administering a ncuroprotectivc steroid in combination or alternation with a vitamin D or a pharmaceutically acceptable salt, ester or prodrug thereof, optionally in a pharmaceutically acceptable carrier to a patient in need thereof, in accordance with any embodiments described above. In certain embodiments, the patient is at risk of or suffering from vitamin D deficiency. In certain other embodiments, the patient is not at risk of vitamin D deficiency.

In particular embodiments, a method is provided to treat a brain injury, including a traumatic brain injury or stroke, in a patient comprising assessing the risk of vitamin D deficiency in the patient, administering a neuroprotective steroid or a pharmaceutically acceptable salt, ester or prodrug thereof, optionally in a pharmaceutically acceptable carrier, to the patient, and administering vitamin D in combination or alternation with a neuroprotective steroid or a pharmaceutically acceptable salt, ester or prodrug thereof, optionally in a pharmaceutically acceptable carrier to an at risk patient. In certain embodiments, vitamin D is administred if the patient is determined to suffer from or be at risk of vitamin D deficiency. In certain embodiments, the neuroprotective steroid is progesterone or allopregnanolone. In certain embodiments, a patient is at risk of vitamin D deficiency. In some embodiments, such a deficiency is determined by the blood serum levels of 25-hydroxy-vitamin D (25-OH-D) in the patient. In some embodiments, a patient is at risk of vitamin D deficiency if the 25-hydroxy-vitamin D (25-OH-D) level in the blood serum is less than 30 ng/ml, less than 20 ng/ml, less than 15 ng/ml or is less than 12 ng/ml. In certain embodiments, a patient is at risk of vitamin D deficiency when the patient is at least 50 years old, or at least 60 years old, or at least 70 years old. Alternatively, a patient can be identified as at risk of vitamin D deficiency by a combination of reduced sun exposure on dark skin pigment.

In certain embodiments, a method of reducing damage from a brain injury or disease is provided wherein a patient is treated with a single dose of a vitamin D in combination with a neuroprotective steroid or a pharmaceutically acceptable salt, ester or prodrug thereof, optionally in a pharmaceutically acceptable carrier, and subsequently is treated with at least one additional dose of neuroprotective steroid. In certain embodiments, the neuroprotective steroid is provided in at least one cycle of therapy, wherein the cycle of therapy comprises administering a therapeutically effective two-level intravenous dosing regime of neuroprotective steroid. The two-level dosing regime can comprise a first time period, wherein a higher hourly dose of neuroprotective steroid is administered to the subject, followed by a second time period, wherein a lower hourly dose of neuroprotective steroid is administered to the subject. In specific methods, the first time period comprises an hourly dose of neuroprotective steroid of about 0.1 mg/kg to about 10 mg/kg, and in particular about 0.1 to about 7.1 mg/kg, the second time period comprises an hourly dose of neuroprotective steroid of about 0.05 mg/kg to about 5 mg/kg, and a third time period comprising a tapered administration protocol is added to the dosing regime. In certain embodiments, the vitamin D is provided at intervals in combination with the neuroprotective steroid, for example the vitamin D can be provided at least once a week in combination with the neuroprotective steroid. In certain other embodiments, the vitamin D is provided at least once a month in combination with the neuroprotective steroid. In separate embodiments, the vitamin D is provided in more than one dose, and is, for example, provided as a daily dosing regimen.

In other embodiments, methods of treating or preventing neurodegeneration resulting from ischemic CNS injuries, in particular from ischemic stroke are provided, comprising administering a vitamin D in combination or alternation with a neuroprotective steroid or a pharmaceutically acceptable salt, ester or prodrug thereof optionally in a pharmaceutically acceptable carrier to a patient in need thereof, in accordance with any embodiments described above. In yet other embodiments, methods of treating or preventing neurodegeneration resulting from hemorrhagic CNS injuries, in particular from hemorrhagic stroke are provided comprising administering a vitamin D in combination or alternation with a neuroprotective steroid to a patient in need thereof, in accordance with any embodiments described above. The methods can alleviate the initial damage to the CNS, in particular to patients at risk of or suffering from a vitamin D deficiency. Therefore, in some embodiments, the compounds are administered to a patient at risk of a CNS injury, in particular to a patient at risk of a stroke. The combinations are also effective at reducing or preventing secondary injuries. Therefore, in other embodiments, the vitamin D and neuroprotective steroid are administered to a patient who has suffered a CNS injury within a window of opportunity after an initital insult. The initial insult can be either a TB1 or a stroke, whether that be an ischemic or hemorrhagic stroke.

In any of the embodiments described herein, the neuroprotective steroid may be represented by formula (I):

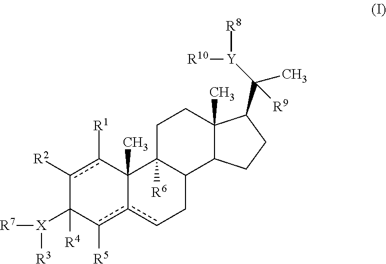

wherein X is O, N or S;

Y is O, N or S;

R¹, R², R⁵ and R⁶ are independently hydrogen, alkyl, halogen, hydroxylcycloalkyl, cycloalkenyl, alkenyl, alkynyl, aryl, alkylaryl, arylalkyl, heterocyclic, heteroaryl, amino, thiol, alkoxy, sulfide, nitro, cyano, azide, sulfonyl, acyl, carboxyl, an ester, an amide, carbamate, carbonate, an amino acid residue or a carbohydrate;

R⁴ is hydrogen or alkyl; or R⁴ and R⁷ together form a double bond;

R³ is hydrogen, optionally substituted acyl, a residue of an amino acid, a carbohydrate, —OR¹¹, —NR¹¹R¹² or R³ is absent;

R⁷ is hydrogen or is absent, or R⁷ together with R⁴ forms a double bond;

R⁸ is hydrogen, optionally substituted acyl, a residue of an amino acid, a carbohydrate, —OR¹¹, —NR¹¹R¹² or R⁸ absent;

R⁹ is hydrogen or alkyl; or R⁹ and R¹⁰ together form a double bond;

R¹⁰ is hydrogen or is absent, or R¹⁰ together with R⁹ forms a double bond;

R¹¹ is the residue of an amino acid, a carbohydrate or an optionally substituted ester or a substituted acyl;

R¹² is hydrogen or alkyl; and

the dotted line indicates the presence of either a single bond or a double bond, wherein the valences of a single bond are completed by hydrogens,

provided that

at least one of XR³R⁷ or YR⁸R¹⁰ is not ═O or OH, and that if the dotted line between C4 and C5 or between C5 and C6 represents a double bond then the other dotted line between C4 and C5 or between C5 and C6 represents a single bond; and with the proviso that neither XR³R⁷ nor YR⁸R¹⁰ represent an ester of aspartic acid, glutamic acid, gama amino butyric acid or a-2-(hydroxyethylamino)-propionic acid; and

with the proviso that when Y is N, R⁸ does not represent aspartic acid, glutamic acid, gama amino butyric acid or a-2-(hydroxyethylamino)-propionic acid.

### BRIEF DESCRIPTION OF THE FIGURES

FIG. 1 shows edema assay data for selected steroid analogues.

FIG. 2A-C show levels of certain inflammatory cytokines in vitamin D deficient and normal animals. A. Uninjured (SHAM) deficient animals show elevated levels of inflammatory cytokines compared to nutritionally normal animals. B. Injured deficient animals treated with vehicle show elevated levels of inflammation at 24 and 72 hours after injury compared to nutritionally normal animals. C. Injured deficient animals treated with PROG also show increased inflammation at 24 and 72 hours compared to normal animals. In B and C, results are normalized to the values for nutritionally normal animals at the same time-point, i.e., all normal values are at value=1 on the vertical axis. Asterisks denote a significant t-test with p<0.05.

FIG. 3A-F shows levels of individual inflammatory cytokines, cleaved caspase-3, and p53 in deficient injured animals under different treatment conditions at 24 and 72 hours after injury. Results are normalized to the vehicle (VH) group within each time-point (vertical axis value=1) and asterisks denote post-hoc p<0.05 significance relative to vehicle. The major treatment effect significantly different from vehicle in most cases is only D+PROG, suggesting a reversal of the injurious effect of deficiency.

FIG. 4A-D shows open-field activity results for normal and deficient animals showing a beneficial effect with combined D+PROG treatment in all cases. Normal animals are shown in darker gray while deficient animals are lighter. All results are normalized to SHAM group results for each nutritional condition. Asterisks denote p<0.05 significance relative to the VH group in each nutritional condition.

FIGS. 5A and 5B are bar graphs showing the effect of PROG on glutamate-induced LDH release (A) and MTT reduction (B) in rat primary cortical neurons. Primary cells were pre-treated with different concentrations of PROG for 24 h and subsequently exposed to glutamate (0.5 μM) for 24 h. PROG was present in the culture medium during the glutamate exposure. The values are expressed as mean±SEM of four experiments. Significant difference #P<0.001 when compared with control; *P<0.001 when compared with vehicle.

FIGS. 6A and 6B are bar graphs showing the effect of VDH on glutamate-induced LDH release (A) and MTT reduction (B) in rat primary cortical neurons. Primary cells were pre-treated with different concentrations of VDH for 24 h and subsequently exposed to glutamate (0.5 μM) for 24 h. VDH was present in the culture medium during the glutamate exposure. The values are expressed as mean±SEM of four experiments. Significant difference #P<0.001 when compared with control; *P<0.001 when compared with vehicle.

FIGS. 7A and 7B are bar graphs showing the effect of combinatorial treatment of PROG and VDH on glutamate-induced LDH release (A) and MTT reduction (B) in rat primary cortical neurons. Primary cells were pre-treated with either best concentrations of PROG and VDH or their combination for 24 h and subsequently exposed to glutamate (0.5 μM) for 24 h. Drugs were present in the culture medium during the glutamate exposure. The values are expressed as mean±SEM of three experiments. Significant difference #P<0.001 when compared with control; *P<0.001 when compared with vehicle.

FIGS. 8A and 8B are bar graphs showing the effect of combinatorial treatment of FROG and VDH on glutamate-induced LDH release (A) and MTT reduction (B) in rat primary cortical neurons. Primary cells were pre-treated with different combinations of PROG and VDH for 24 h and subsequently exposed to glutamate (0.5 μM) for 24 h. Drugs were present in the culture medium during the glutamate exposure. The values are expressed as mean±SEM of four experiments. Significant difference #P<0.001 when compared with control; *P<0.001 when compared with vehicle; and §P<0.01 when compared with P20 group.

FIGS. 9A and 9B show the effect of PROG and VDH exposure on the activation of MAPK in primary cortical neurons. Cells were exposed to hormones either separately or in different combinations for 30 min. Cells were lysed after incubation and lysates were separated on 12.5% SDS gel and transferred onto PVDF membrane. The membrane was probed with either phosphor-ERK1/2 or total ERK1/2 protein (FIG. 9A). Phospho-ERK1/2 data were normalized with total ERK1/2 protein. Data were analyzed using analysis of variance (ANOVA) and Neuman-Keuls test. Values are expressed as mean±standard error of the mean (SEM) of three independent experiments. Significant difference *P<0.05 when compared with control; †P<0.05 as compared to PROG (20 μM) and VDH (20 nM) groups. Values in parenthesis represent fold increase in MAPK over control values (FIG. 9B).

FIG. 10 is a diagram showing brain injury processes affected by PROG and VDH.

### DETAILED DESCRIPTION OF THE INVENTION

Provided herein are methods of treatment and compositions for treatment of patients suffering from nervious system damage, in particular due to inflammatory reactions to injury or disease, and particularly for patients also at risk of suffering from a vitamin D deficiency.

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

### I. VITAMIN D

Vitamin D and related compounds are classed as secosteroids (a steroid in which one of the bonds in the steroid rings is broken). These compounds can exert steroid-like effects throughout the body, directly affecting the expression of over 1,000 genes (Eelen et al., 2004) through the nuclear vitamin D receptor (VDR).

Several forms of vitamin D have been discovered naturally occurring, and a variety of secosteroid analogs have been synthetically designed. The two major forms of vitamin D in nature are vitamin D₂ or ergocalciferol, and vitamin D₃ or cholecalciferol. These are known collectively as calciferol. The structural difference between vitamin D₂ and vitamin D₃ is in their side chains. The side chain of D₂ contains a double bond between carbons 22 and 23, and a methyl group on carbon 24. Vitamin D₂ is derived from fungal and plant sources, and is not produced by the human body. Vitamin D₃ is derived from animal sources and is made in the skin when 7-dehydrocholesterol reacts with UVB ultraviolet light at wavelengths between 270-300 nm, with peak synthesis occurring between 295-297 nm. These wavelengths are present in sunlight when the UV index is greater than 3. At this solar elevation, which occurs daily within the tropics, daily during the spring and summer seasons in temperate regions, and almost never within the arctic circles, adequate amounts of vitamin D₃ can be made in the skin after only ten to fifteen minutes of sun exposure at least two times per week to the face, arms, hands, or back without sunscreen. With longer exposure to UVB rays, an equilibrium is achieved in the skin, and the vitamin simply degrades as fast as it is generated.

In humans, D₃ is as effective as D₂ at increasing the levels of vitamin D hormone in circulation, although certain reports state that D₃ is more effective than D₂. However, in some species, such as rats, vitamin D₂ is more effective than D₃.

The chemical structure of naturally occurring vitamin D2 is:

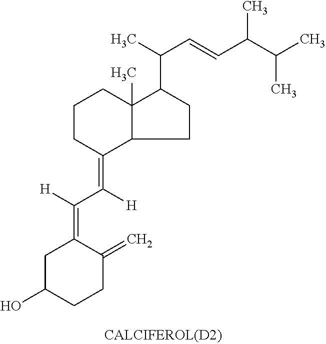

and of vitamin D3 is:

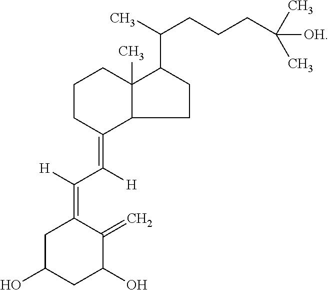

Additional forms of vitamin D that have been discovered include:

| | | |
|---|---|---|
| Vitamin | molecular compound of ergocalciferol | |
| D₁ | with lumisterol, 1:1 | |
| Vitamin D₂ | ergocalciferol (made from ergosterol) | 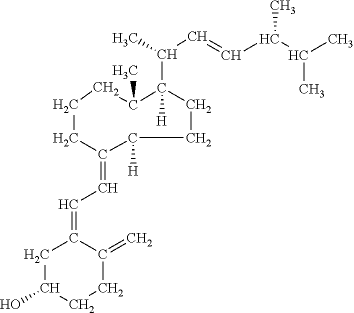 |
| Vitamin D₃ | cholecalciferol (made from 7- dehydrocholesterol in the skin). | 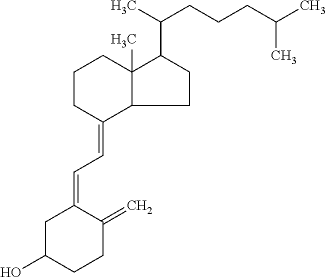 |
| Vitamin D₄ | 22-dihydroergocalciferol | 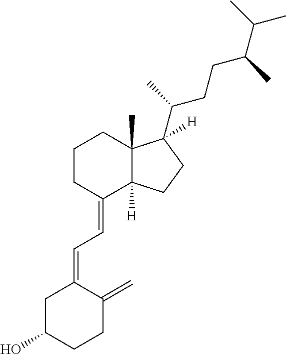 |
| Vitamin D₅ | sitocalciferol (made from 7- dehydrositosterol | 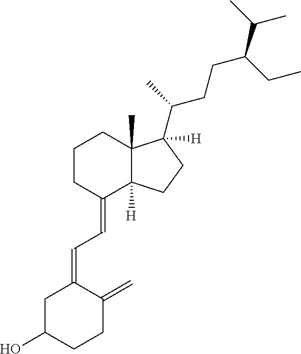 |

In one embodiment, the vitamin D in the methods and compositions of the invention has the structure:

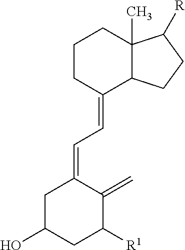

wherein R is alkyl, alkenyl, alkynyl, heteroalkyl, haloalkyl, aryl, heteroaryl or heterocyclyl and wherein each R group may be optionally substituted with one or more of hydroxy, alkoxy, fluoro, chloro, bromo, iodo, CF₃, alkenyl, alkynyl, alkyl, aryl, heteroaryl or heterocycly groups; and

R¹ is H, alkyl or hydroxy.

In another embodiment, the vitamin D is seocalcitol. Seocalcitol (aka CB1089) is courently being tested by Cougar Biotechnology for its potential as an anticancer agent. Seocalcitol is an analog of calcitriol that has been shown, in pre-clinical cancer studies, to be 50-200 times more potent than calcitriol with respect to regulation of cell growth and differentiation in cancer studies. Importantly, pre-clinical studies also indicate that seocalcitol has reduced calcemic activity compared to calcitriol, significantly reducing the incidence of hypercalcemia. The chemical structure is 5-(2-(1-(6-ethyl-6-hydroxy-1-methyl-octa-2,4-dienyl)-7a-methyl-octahydro-inden-4-ylidcnc)-ethylidene)-4-methylene-cyclohexane-1,3-diol.

Certain additional secosteroid or vitamin D analogs are described in U.S. Pat. Nos. 4,996,318, 5,763,234 and 5,789,399. Certain vitamin D analogs include the following:

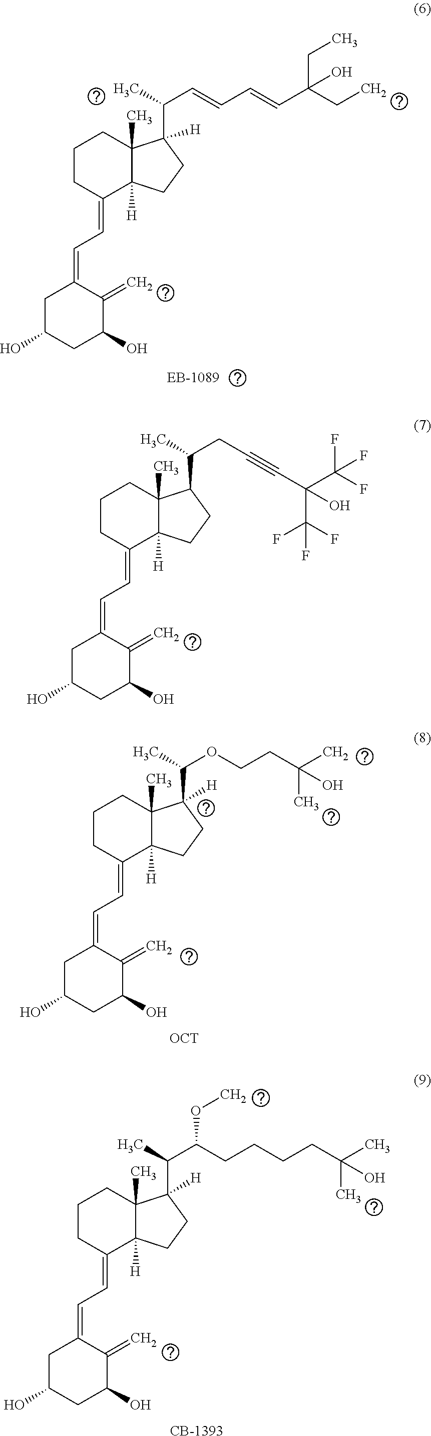

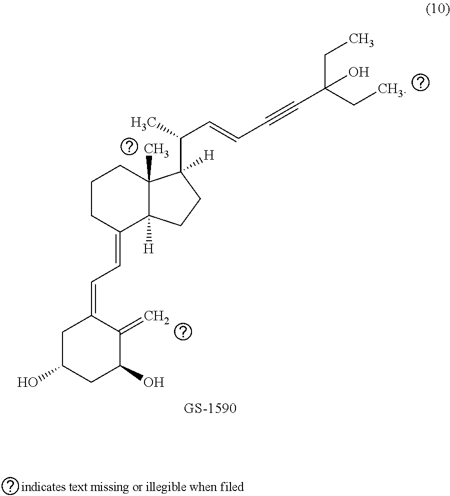

In supplements and fortified foods, vitamin D is available in two forms, D₂ (ergocalciferol) and D₃ (cholecalciferol). Both vitamin D₂ and D₃ are used for human nutritional supplementation, and pharmaceutical forms include calcitriol (1α,25-diOH-D), doxercalciferol and calcipotriene. Vitamin D₂ is manufactured by the UV irradiation of ergosterol in yeast, and vitamin D₃ is manufactured by the irradiation of 7-dehydrocholesterol from lanolin and the chemical conversion of cholesterol. The two forms have traditionally been regarded as equivalent based on their ability to cure rickets, but evidence has been offered that they are metabolized differently. Vitamin D₃ could be more than three times as effective as vitamin D₂ in raising serum 25-OH-D concentrations and maintaining those levels for a longer time, and its metabolites have superior affinity for vitamin D-binding proteins in plasma. Both forms (as well as vitamin D in foods and from cutaneous synthesis) effectively raise serum 25-OH-D levels. In certain embodiments of the invention, the vitamin D of the invention is a vitamin D3 analog. In certain other embodiments, the vitamin D formulation is paricalcitol.

Vitamin D is also both activated by and has direct effects in the CNS (Garcion et al., 2002). The nuclear receptor for vitamin D has been localized in neurons and glial cells and genes encoding the enzymes involved in the metabolism of this hormone are also expressed in brain cells. The reported biological effects of vitamin D in the nervous system include the biosynthesis of neurotrophic factors and at least one enzyme involved in neurotransmitter synthesis, inhibition of the synthesis of inducible nitric oxide synthase and increase glutathione levels. Certain neuroprotective and immunomodulatory effects of this hormone have been described in experimental models of neurodegenerative and neuroimmune diseases. It has been shown to affect certain systems similar to those modulated by certain neurostcroids, in particular estradiol-like compounds (Losem-Heinrichs et al., 2005; Ray and Gupta (2006) Drugs Put. 31:65).

Vitamin D is highly susceptible to oxidation. Therefore, the compound should be formulated in a way that protects the active ingredient from oxidizing. In certain embodiments, the vitamin D is provided as a cross-linked formulation, with an appropriate polymer. In other embodiments, the compound is provided in a microcncapsulated formulation. In certain embodiments, the compound is in a microsphere or microbead formulation for enhanced stability, and in certain instances for extended release. Vitamin D supplements are available over the counter in certain formulations. For example, calcitriol is a form of vitamin D that is used to treat and prevent low levels of calcium in the blood of patients whose kidneys or parathyroid glands are not working normally. Calcitriol comes as a capsule and a solution (liquid) to take by mouth. Calcitriol is also sometimes used to treat rickets (softening and weakening of bones in children caused by lack of vitamin D), osteomalacia (softening and weakening of bones in adults caused by lack of vitamin D), and familial hypophosphatemia (rickets or osteomalacia caused by dccreascd ability to break down vitamin D in the body). Calcitriol is also sometimes used to increase the amount of calcium in the blood of premature (born early) babies.

### II. ASSESSING VITAMIN D DEFICIENCY

In certain embodiments of the invention, a patient is assessed for a risk of vitamin D deficiency or vitamin D insufficiency. Certain secondary indicia of risk include age, darker skin color or if the person lives in a northern climate. A prolonged deficiency of vitamin D in adults results in osteomalacia and in children in rickets. Both diseases involve defects in bones. Vitamin D deficiency can be caused by conditions that result in little exposure to sunlight. These conditions include: living in northern countries; having dark skin; being elderly or an infant, and having little chance to go outsidc; and covering one's face and body, such as for religious reasons. Most foods contain little or no vitamin D. As a result, sunshine is often a deciding factor in whether vitamin D deficiency occurs. Although fortified milk and fortified infant formula contain high levels of vitamin D, human breast milk is rather low in the vitamin.

Vitamin D levels are usually determined by measuring the blood serum levels of 25-OH-D. In some embodiments, levels of 25-OH-D below 25 nmol/L are defined as vitamin D deficiency, levels between 25 nmol/L and 50 nmol/L are defined as insufficiency, and blood serum levels of 25-OH-D higher than 50 nmoVL are defined as normal. In other embodiments, a normal blood serum concentration of 25-OH-D is 25-50 ng/ml. However, a patient can be at risk of deficiency if a blood serum level is less than about 30 ng/ml. In some embodiments, a vitamin D deficiency is correlated with clinical symptoms or disease, such osteomalacia or rickets, while a vitamin D insufficiency is not correlated with any disease, although it may be correlated with clinical symptoms. In some embodiments, a vitamin D insufficiency is defined as a vitamin D level between a level indiciative of vitamin D deficiency and a level deemed healthy or normal.

In one particular embodiment, a method is provided to treat a brain injury, including a traumatic brain injury or stroke, in a patient comprising assessing the risk of vitamin D deficiency in the patient, administering a neuroprotective steroid to the patient and administering vitamin D in combination with progesterone to an at risk patient. In certain embodiments, the neuroprotective steroid is progesterone or allopregnanolone. In certain embodiments, a patient is at risk of vitamin D deficiency if a measurement of 25-hydroxy-vitamin D (25-OH-D) in the blood serum is less than 30 ng/ml. In other embodiments, a patient is at risk of vitamin D deficiency if a measurement of 25-OH-D in the blood serum is less than 20 ng/ml, or is less than 15 ng/ml or is less than 12 ng/ml. In certain embodiments, a patient at risk of vitamin D deficiency is at least 50 years old, or at least 60 years old, or at least 70 years old. Alternatively, a patient can be identified as at risk of vitamin D deficiency by a combination of reduced sun exposure on dark skin pigment.

Vitamin D deficiency can be directly diagnosed by measuring the level of 25-hydroxy-vitamin D in the blood serum. 25-OH-D is not the active form of the vitamin. It must be converted to 1,25-diOH-D in order to cause responses in various organs of the body. However, the levels of vitamin D, or of 1,25-diOH-D in the blood, do not give a reliable picture of whether a person is deficient in the vitamin. For this reason, they typically are not measured when testing for vitamin D deficiency.

In certain embodiments, a patient is at risk of vitamin D deficiency if a measurement of 25-OH-D in the blood serum is less than 30 ng/ml. In other embodiments, a patient is at risk of vitamin D deficiency if a measurement of 25-OH-D in the blood serum is less than 20 ng/ml, or is less than 15 ng/ml or is less than 12 ng/ml. Patients at risk of vitamin D deficiency can be identified as patients above 50 years old, or above 60 years old, or above 70 years old. Alternatively, patients are identified as at risk of vitamin D deficiency by assessment of a combination of their location, skin color and age.

Several blood test proccdurcs have been tried over the years to predict vitamin D levels through indirect measures of related blood chemistry. One research study (Singh, et. al., Journal of Orthopaedic Surgery 2004; 12(1):31-34) demonstrated that using routine blood hone chemistry tests for “plasma calcium, alkaline phosphatase, and phosphate cannot detect vitamin D insufficiency”. One must measure blood levels of vitamin D directly.

Blood tests are available that measure the two forms of vitamin D: 25-OH-D (circulating) and 1,25-diOH-D (active). Each test is given for specific diagnostic purposes. They can be used to monitor disease state or supplement effects. Whatever test is used, it must be measured and interpreted accurately. Recent studies have shown that some testing methods give inaccurate results (Garland, et. al. Int. J. Epidemiol. 2006 April; 35(2):217-20) and the mistaken belief that vitamin D levels are normal when in reality they are much lower. Two main types of 25-OH-D assays are available, based on either high-performance liquid chromatography with UV or mass detection or higher throughput kits based on protein (competitive protein binding assay or radioimmunoassay) binding. Both assays may be used for testing blood serum levels (for review of available techniques see Jones, et al. (2007) J. Bone Miner. Res. 22 Supp 2: V11-5).

In accordance with any embodiments described herein, a vitamin D insufficiency may be assessed and/or treated in the same manner as a vitamin D deficiency.

### III. NEUROPROTECTIVE STEROIDS

The invention provides improved methods and compositions for treatment of neural injury and inflammation, particularly in patients deficient in, or at risk of deficiency in, vitamin D. The treatment of neural injuries with certain neuroprotective steroids can effectively reduce secondary damage and improve therapeutic outcome, however in certain patients these compounds are not effective. It has been found that combination therapy with a vitamin D enhances efficacy of the combination and provides improved therapeutic outcome over administration of either substance alone.

The term “neuroprotective steroid” as used herein is intended to encompass progesterone as well as prodrugs, analogues of progesterone, analogues of progesterone metabolites or derivatives and other non-progestin steroid compounds that exhibit in vivo efficacy in the methods described herein, and/or that exhibit efficacy in the in vitro assays described herein. Exemplary neuroprotective steroids include those described herein and in U.S. provisional application 61/032,315, U.S. provisional application 61/031,629, U.S. provisional application 61/031,567, U.S. provisional application 61/148,811 and PCT application PCT/US2009/03533, each of which is incorporated herein by reference in its entirety. In some embodiments, the neuroprotective steroids of the invention exhibit increased solubility in aqueous solvents and are capable of forming pharmaceutically acceptable salts that further increase their aqueous solubility as compared to a reference steroid, such as progesterone. As used herein, a prodrug designates a neuroprotective steroid that is administered in an inactive or less active form and that, once administered, is metabolized in vivo into an active form. In some embodiments, the prodrug may provide improved solubility, absorption, distribution, metabolism, and/or excretion as compared to the reference drug. Also provided are pharmaceutical compositions comprising the neuroprotective steroid, pharmaceutically acceptable salts, esters or prodrugs thereof, and methods for the treatment or prevention of nervous system injuries, CNS injuries, including traumatic brain injury and stroke, and other injuries as described herein above and below.

In particular embodiments, the present invention relates to neuroprotective steroids that comprise amino acid residues, carbohydrates or other suitable polar groups at the 3- and/or 20-positions of the steroid ring system. The improved water solubility of certain neuroprotective steroids described herein can facilitate the administration of the compounds, in particular intravenous administration, which provides the fastest possible exposure of the active agent to the brain or other CNS sites where it is needed, increasing the efficacy of the drug. In addition, the neuroprotective steroids will minimize undesired side effects that are typically accompany acute or prolonged treatment with progesterone, such as sleepiness, reduced arousal and increased blood clotting.

Certain progestins useful in the present methods and compositions include progesterone, 5-dehydroprogesterone, 6-dehydro-retroprogesterone(dydrogesterone), allopregnanolone (allopregnan-3α, or 3β-ol-20-one), ethynodiol diacctatc, hydroxyprogesterone caproate (pregn-4-ene-3,20-dione, 17-(1-oxohexy)oxy); levonorgestrel, norethindrone, norethindrone acetate (19-norpregn-4-en-20-yn-3-one, 17-(acetyloxy)-,(17α)-); norethynodrel, norgestrel, pregnenolone, and megestrol acetate. Useful compounds also can include allopregnone-3α or 3β, 20α or 20β-diol (see Merck Index, 12th ed., 266-286); allopregnane-3β, 21-diol-11,20-dione; allopregnane-3β, 17α-diol-20-one; 3,20-allopregnanedione, allopregnane, 3β, 11β, 17α,20β,21-pentol; allopregnane-3β, 17α,20β, 21-tetrol; allopregnane-3α or 3β, 11β, 17α,21-tetrol-20-one, allopregnane-3β, 17α,20α or 200-trial; allopregnane-3β, 17α, 21-triol-11,20-dione; allopregnane-3β, 11β,21-triol-20-one; allopregnane-3β, 17α, 21-triol-20-one; allopregnane-3α or 3β-ol-20-one; pregnanediol; 3,20-pregnanedione; pregnari-3α-ol-20-one; 4-pregnene-20,21-diol-3,11-dione; 4-pregnene-11β, 17α,20β,21-tetrol-3-one; 4-pregnene-17α,20β,21-triol-3,11-dione; 4-pregnene-17α,20β,21-triol-3-one, and pregnenolonc methyl ether, as well as derivatives thereof such as esters with non-toxic organic acids such as acetic acid, benzoic acid, maleic acid, malic acid, caproic acid, citric acid and the like.

In one embodiment, the neuroprotective steroid is ganaxolone (3a-hydroxy-3b-methyl-5a-pregnan-20-one). This compound is a 3b-methylated synthetic analog of the neurosteroid allopregnanolone (3a,5a-P), a metabolite of progesterone. Importantly, ganaxolone does not have significant classical nuclear steroid hormone activity and, unlike 3a,5a-P, cannot be converted to metabolites with such activity. Phase 1 and Phase 2 human trials indicate that ganaxolone is well tolerated and that it may be efficacious in the treatment of diverse forms of epilepsy in children and adults in the description of the steroids. This compound is being developed by Marinus Pharmaceuticals.

Progesterone itself is lipid-soluble and essentially water insoluble. Therefore, in certain embodiments, the compound is a neuroprotective steroid that comprises polar groups and exhibit increased aqueous solubility. In certain embodiments, the progesterone analogs are neuroprotective steroids functionalized with polar groups at the C3 and C20 positions that exhibit greater water solubility than the parent compounds and are useful for the prevention and treatment of central nervous system injury, particularly traumatic brain injury and stroke. In one embodiment, the neuroprotective steroids of the invention are derivatized at the 3- and/or 20-positions of the steroid ring to yield analogs that comprise polar amino acid substitutents capable of forming water soluble salts. In other embodiments, the neuroprotective steroids are derivatized at the 3- and/or 20-positions a carbohydrate or a substituted acyl group. The neuroprotective steroids are optionally substituted with non-hydrogen substituents at the 9-, 1-, 2-, 3-, and 4-positions and may contain double bonds between C1 and C2, C4 and C5 and between C5 and C6. The amino acids may be either the naturally occurring or synthetic amino acids in either the D, L configuration or may be a mixture of D and L forms.

In one embodiment, analogues of steroid compounds are provided that are modified at the 3- and/or 20-position of the steroid ring system to incorporate polar groups. The ring numbering shown below for the structure of progesterone is maintained throughout this document to avoid ambiguity.

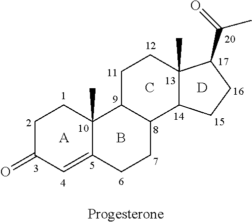

Substituents on the neuroprotective steroids that lie below the plane of the paper as drawn are termed in the “α” or “alpha” configuration. Substituents that lie above the plane of the paper are termed in the “β” or “beta” configuration. For example the two methyl groups shown in the progesterone structure below are in the beta configuration.

In one embodiment of the invention are provided steoid analogues, such as progesterone, pregnenolone and the like, comprising an amino acid residue, a carbohydrate or other polar group bonded to the 3-position of the steroid ring system. In another embodiment of the invention, neuroprotective steroids that comprise an amino acid residue, a carbohydrate or other polar group bonded to the 20-position of the ring system are provided. In still another embodiment, neuroprotective steroids comprising amino acid residues and/or carbohydrates or other polar groups at the 3- and at the 20-positions of the ring system are provided. These neuroprotective steroids have greater aqueous solubility than the parent compounds and are thus advantageous for administration, in particular in situations in which rapid availability and effective dosing of the compounds are critical. In some embodiments, the neuroprotective steroids comprise a basic nitrogen group that enables the formation of pharmaceutically acceptable salts and prodrugs. The neuroprotective steroids are useful for the treatment or prevention of central nervous system injury, particularly traumatic brain injury and stroke.

In one embodiment, the neuroprotective steroid has the Formula I:

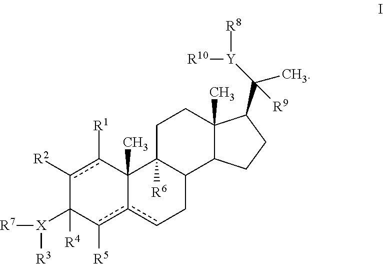

wherein X is O, N or S;

Y is O, N or S;

R¹, R², R⁵ and R⁶ are independently hydrogen, alkyl, halogen, hydroxylcycloalkyl, cycloalkenyl, alkenyl, alkynyl, aryl, alkylaryl, arylalkyl, heterocyclic, heteroaryl, amino, thiol, alkoxy, sulfide, nitro, cyano, azide, sulfonyl, acyl, carboxyl, an ester, an amide, carbamate, carbonate, an amino acid residue or a carbohydrate;

R⁴ is hydrogen or alkyl; or R⁴ and R⁷ together form a double bond;

R³ is hydrogen, optionally substituted acyl, a residue of an amino acid, a carbohydrate, —OR¹¹, —NR¹¹R¹² or R³ is absent;

R⁷ is hydrogen or is absent, or R⁷ together with R⁴ forms a double bond;

R⁸ is hydrogen, optionally substituted acyl, a residue of an amino acid, a carbohydrate, —OR¹¹, —NR¹¹R¹² or R⁸ absent;

R⁹ is hydrogen or alkyl; or R⁹ and R¹⁰ together form a double bond;

R¹⁰ is hydrogen or is absent, or R¹⁰ together with R⁹ forms a double bond;

R¹¹ is the residue of an amino acid, a carbohydrate or an optionally substituted ester;

R¹² is hydrogen or alkyl; and the dotted line indicates the presence of either a single bond or a double bond, wherein the valences of a single bond are completed by hydrogens, provided that at least one of XR³R⁷ or YR⁸R¹⁰ is not ═O or OH; and that if the dotted line between C4 and C5 or between C5 and C6 represents a double bond then the other dotted line between C4 and C5 or between C5 and C6 represents a single bond; and with the proviso that neither XR³R⁷ nor YR⁸R¹⁰ represent an ester of aspartic acid, glutamic acid, gama amino butyric acid or a-2-(hydroxyethylamino)-propionic acid; and with the proviso that when Y is N, R⁸ does not represent aspartic acid, glutamic acid, gama amino butyric acid or a-2-(hydroxyethylamino)-propionic acid.

In one embodiment, R¹, R², R⁵ and R⁶ are independently hydrogen, alkyl, halogen or hydroxyl.

In another embodiment, R¹, R², R⁵ and R⁶ are independently methyl, ethyl or propyl.

In still another embodiment, R¹, R², R⁵ and R⁶ are independently thiomethyl, hydroxymethyl or cyano.

In another embodiment, R¹, R², R⁵ and R⁶ are independently vinyl or ethynyl.

In still another embodiment, R¹, R², R⁵ and R⁶ are independently fluoro, bromo, chloro or iodo.

In still another embodiment, R⁴ and R⁹ are independently hydrogen or methyl.

In some embodiments, X and Y are O. In other embodiments, X is O and Y is N or X is N and Y is O. In other embodiments, both X and Y are N. In certain embodiments in which Y is O, R⁹ and R¹⁰ come together to form a double bond.

In certain embodiments, one of R³ and R⁸ is a residue of an amino acid. In particular embodiments, the amino acid is a naturally occurring amino acid. In certain embodiments, R³ is a residue of an amino acid. In certain other embodiments, R⁸ is a residue of an amino acid. In yet further embodiments, both R³ and R⁸ are residues of an amino acid.

In one embodiment of Formula I, X is O, R³ is the residue of an amino acid, and R⁷ is absent.

In another emobidment of Formula I, Y is O, R⁸ is the residue of an amino acid, and R¹⁰ is absent;

In another embodiment of Formula I, X is N; R⁷ together with R⁴ form a double bond; R³ is OR¹¹ or NR¹¹R¹²; and R¹¹ is the residue of an amino acid.

In another embodiment of Formula I, Y is N; R¹⁰ together with R⁹ form a double bond; R⁸ is OR¹¹ or NR¹¹R¹²; and R¹¹ is the residue of an amino acid.

In another embodiment of Formula I, X is O; R³ is the residue of a naturally occurring amino acid; R⁷ is absent; Y is O; R⁸ is absent; and R⁹ and R¹⁰ together form a double bond.

In still another embodiment, Y is O; R⁸ is the residue of a naturally occurring amino acid; R¹⁰ is absent; X is O; R⁷ is absent; and R³ and R⁴ together form a double bond.

In another embodiment of Formula I, X is O; R³ is the residue of an amino acid; R⁷ is absent; Y is N; R¹⁰ together with R⁹ form a double bond; R⁸ is OR¹¹ or NR¹¹R¹²; and R¹¹ is the residue of an amino acid.

In yet another embodiment of Formula I, X is N; R⁷ together with R⁴ form a double bond; R³ is OR¹¹ or NR¹¹R¹²; R¹¹ is the residue of an amino acid; Y is O; R⁸ is the residue of an amino acid; and is absent.

In another embodiment of Formula I, X is O; R³ is the residue of an amino acid; R⁷ is absent; Y is O, R⁸ is the residue of an amino acid, and R¹⁰ is absent.

In yet another embodiment of Formula I, X is N; R⁷ together with R⁴ form a double bond; R³ is OR¹¹ or NR¹¹R¹²; Y is N; R¹⁰ together with R⁹ form a double bond; R⁸ is OR¹¹ or NR¹¹R¹²; and R¹¹ is the residue of an amino acid.

In yet another embodiment of Formula I, X is O; R³ is the residue of an amino acid; R⁷ is absent; R¹, R², R⁴, R⁵ and R⁶ are independently hydrogen, alkyl, halogen or hydroxyl.

In yet another embodiment of Formula I, X is O; R³ is the residue of an amino acid; R⁷ is absent; R¹, R², R⁴, R⁵ and R⁶ are hydrogen.

In another embodiment of Formula I, Y is O; R⁸ is the residue of an amino acid; R¹⁰ is absent; R¹, R², R⁴, R⁵ and R⁶ are independently hydrogen, alkyl, halogen or hydroxyl.

In still another embodiment of Formula I, Y is O; R⁸ is the residue of an amino acid; R¹⁰ is absent; R¹, R², R⁴, R⁵ and R⁶ are hydrogen.

In one embodiment of Formula I, the dotted line between C4 and C5 represents a single bond and the dotted line between C5 and C6 represents a single bond.

In another embodiment of Formula I, the dotted line between C4 and C5 represents a single bond and the dotted line between C5 and C6 represents a double bond.

In another embodiment of Formula I, the dotted line between C4 and C5 represents a double bond and the dotted line between C5 and C6 represents a single bond.

In still another embodiment, the dotted line between C1 and C2 represents a single bond. In another embodiment, the dotted line between C1 and C2 represents a double bond.

In certain embodiments of Formula I, a residue of an amino acid is connected to the steroid ring system at the carboxyl group of the amino acid. In other embodiments, a residue of an amino acid is connected to the steroid at the amino acid side chain. For example, amino acids that contain side chains with functional groups that are capable of forming a bond with a hydroxy or a ketone group may be boded to the steroid ring by such a group. In other embodiments, the reactive groups on the amino acid side chains may displace leaving groups formed on the steroid moiety to form a covalent bond. Non-limiting examples of amino acids with reactive groups in the side chain include lysine, cysteine, serine, tyrosine, aspartic acid, arginine and the like.

The amino acid(s) in any of the embodiments of the invention described herein may be naturally occurring or synthetic amino acids and may be in the D or L stereoisomeric form or may exist as a D, L mixture. For example the 20 naturally occurring α-amino acids in the L-configuration are encompassed by the invention as well as β-amino acids in the D-configuration. Synthetic amino acids in either stereoisomeric form are also encompassed.

In another embodiment, the enantiomers of the compounds of Formula I are provided. In this embodiment, the stereochemical configuration of each asymmetric carbon is opposite that of the natural steroids and analogues of the natural steroids. For example, the configuration of C9, C10, C13 and C17 carbon atoms would be opposite to the configuration as drawn in the structure above.

In another embodiment, a neuroprotective steroid of Formula II is provided:

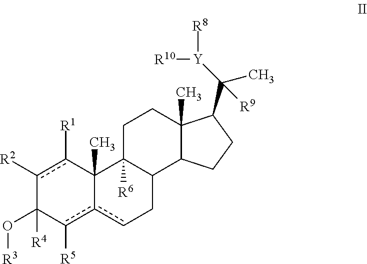

wherein Y is O, N or S;

R¹, R², R⁵ and R⁶ are independently hydrogen, alkyl, halogen, hydroxylcycloalkyl, cycloalkenyl, alkenyl, alkynyl, aryl, alkylaryl, arylalkyl, heterocyclic, heteroaryl, amino, thiol, alkoxy, sulfide, nitro, cyano, azide, sulfonyl, acyl, carboxyl, an ester, an amide, carbamate, carbonate, an amino acid residue or a carbohydrate;

R³ is hydrogen, optionally substituted acyl, a residue of an amino acid or a carbohydrate;

R⁴ is hydrogen or alkyl;

R⁸ is hydrogen, optionally substituted acyl, a residue of an amino acid, a carbohydrate, —O¹¹, —NR¹¹R¹² or R⁸ is absent;

R⁹ is hydrogen or alkyl; or R⁹ and R¹⁰ together form a double bond;

R¹⁰ is hydrogen or absent, or R¹⁰ together with R⁹ form a double bond;

R¹¹ is the residue of an amino acid, a carbohydrate or optionally substituted acyl;

R¹² is hydrogen or alkyl; and the dotted lines indicate the presence of either a single bond or a double bond, wherein the valences of a single bond are completed by hydrogens, provided that if the dotted line between C4 and C5 or between C5 and C6 represents a double bond then the other dotted line between C4 and C5 or between C5 and C6 represents a single bond; and with the proviso that neither R³ nor YR⁸R¹⁰ represent an ester of aspartic acid, glutamic acid, gama amino butyric acid or a-2-(hydroxyethylamino)-propionic acid; and with the proviso that when Y is N, R⁸ does not represent aspartic acid, glutamic acid, gama amino butyric acid or a-2-(hydroxyethylamino)-propionic acid.

In some embodiments, Y is O. In other embodiments, Y is N. In certain embodiments in which Y is O, R⁹ and R¹⁰ come together to form a double bond. In certain embodiments, one of R³ and R⁸ is a residue of an amino acid. In particular embodiments, the amino acid is a naturally occurring amino acid. In certain embodiments, R³ is a residue of an amino acid. In certain other embodiments, R⁸ is a residue of an amino acid. In yet further embodiments, both R³ and R⁸ are residues of an amino acid.

In one embodiment, R¹, R², R⁵ and R⁶ are independently hydrogen, alkyl, halogen or hydroxyl.

In another embodiment, R¹, R², R⁵ and R⁶ are independently methyl, ethyl or propyl.

In still another embodiment, R¹, R², R⁵ and R⁶ are independently thiomethyl, hydroxymethyl or cyano.

In another embodiment, R¹, R², R⁵ and R⁶ are independently vinyl or ethynyl.

In still another embodiment, R¹, R², R⁵ and R⁶ are independently fluoro, bromo, chloro or iodo.

In still another embodiment, R⁴ and R⁹ are independently hydrogen or methyl.

In one embodiment of Formula II, Y is O; R⁸ is the residue of an amino acid; and R¹⁰ is absent.

In another embodiment of Formula II, Y is N; R¹⁰ together with R⁹ form a double bond; R⁸ is OR¹¹; and R¹¹ is the residue of an amino acid.

In another embodiment of Formula II, Y is N; R¹⁰ together with R⁹ form a double bond; R⁸ is —NR¹¹R¹²; R¹¹ is the residue of an amino acid; and R¹² is hydrogen.

In another embodiment of Formula II, R³ is the residue of a naturally occurring amino acid; R⁴ is hydrogen; Y is O; R¹⁰ together with R⁹ form a double bond; and R⁸ is absent.

In another embodiment of Formula II, R³ is a carbohydrate; R⁴ is hydrogen; Y is O; R¹⁰ together with R⁹ form a double bond; and R⁸ is absent.

In another embodiment of Formula II, R³ is the residue of a naturally occurring amino acid; R⁴ is hydrogen; Y is O; R⁸ and R⁹ are hydrogen; and R¹⁰ is absent.

In another embodiment of Formula II, R⁶ is alkyl or fluoro. In yet another embodiment of Formula II, R¹, R² and R⁵ are independently hydrogen or alkyl.

In another embodiment, R¹ and R² are hydroxyl. In still another embodiment, R¹ and R² are independently hydroxyl or halogen. In another embodiment of Formula II, R¹ is alkyl; and R² and R⁵ are hydrogen. In another embodiment, of Formula II, R² is alkyl; and R¹ and R⁵ are hydrogen. In still another embodiment, of Formula II, R⁵ is alkyl; and R¹ and R² are hydrogen.

In another embodiment of Formula II, R³ is the residue of a naturally occurring amino acid; R⁴ is hydrogen; Y is O; R¹⁰ together with R⁹ form a double bond; R⁸ is absent; and R¹, R², R⁵ and R⁶ are hydrogen.

In another embodiment of Formula II, R³ is the residue of a naturally occurring amino acid; R⁴ is alkyl; Y is O; R¹⁰ together with R⁹ form a double bond; R⁸ is absent; and R¹, R², R⁵ and R⁶ are hydrogen.

In one embodiment of Formula II, the dotted line between C4 and C5 represents a single bond and the dotted line between C5 and C6 represents a single bond.

In another embodiment of Formula II, the dotted line between C4 and C5 represents a single bond and the dotted line between C5 and C6 represents a double bond.

In another embodiment of Formula II, the dotted line between C4 and C5 represents a double bond and the dotted line between C5 and C6 represents a single bond.

In still another embodiment of Formula II, the dotted line between C1 and C2 represents a single bond. In still another embodiment, the dotted line between C1 and C2 represents a double bond.

In another embodiment of Formula II, OR³ is in the alpha configuration. In still another embodiment, OR³ is in the beta configuration.

In one embodiment of Formula II, the dotted lines between C4 and C5 and between C5 and C6 represent a single bond, and the hydrogen at the C5 bridgehead carbon is in the alpha configuration. In another embodiment, the dotted lines between C4 and C5 and between C5 and C6 represent a single bond, and the hydrogen at the C5 bridgehead carbon is in the beta configuration

In one embodiment of Formula II, R³ represents a naturally occurring α-amino acid in the L-configuration. In another embodiment, R³ is a residue of L-valine. In other embodiments, R³ represents an amino acid residue with the D-configuration or R³ represents a non-natural amino acid. In other embodiments, R³ represents the residue of a β γ or δ amino acid.

In one preferred embodiment of Formula II, R³ represents an ester of an amino acid. In another embodiment, R³ represents an ester of an amino acid residue where the ester bond is formed with a carboxylate group on the side chain of the amino acid. In certain embodiments of Formula II, a residue of an amino acid is connected to the steroid ring system at the carboxyl group of the amino acid. In other embodiments, a residue of an amino acid is connected to the steroid at the amino acid side chain. For example, amino acids that contain side chains with functional groups that are capable of forming a bond with a hydroxy or a ketone group may be boded to the steroid ring by such a group. In other embodiments, the reactive groups on the amino acid side chains may displace leaving groups formed on the steroid moiety to form a covalent bond. Non-limiting examples of amino acids with reactive groups in the side chain include lysine, cysteine, serine, tyrosine, aspartic acid, arginine and the like.

The amino acid(s) in any of the embodiments of the invention described herein may be naturally occurring or synthetic amino acids and may be in the D or L stereoisomeric form or may exist as a D, L mixture. For example the 20 naturally occurring α-amino acids in the L-configuration are encompassed by the invention as well as β-amino acids in the D-configuration. Synthetic amino acids in either stereoisomeric form are also encompassed.

In another embodiment, the enantiomers of the compounds of Formula II are provided. In this embodiment, the stereochemical configuration of each asymmetric carbon is opposite that of the natural steroids and analogues of the natural steroids. For example, the configuration of C9, C10, C13 and C17 carbon atoms would be opposite to the configuration as drawn in the structure above.

In another embodiment, a progesterone analogue of Formula III is provided:

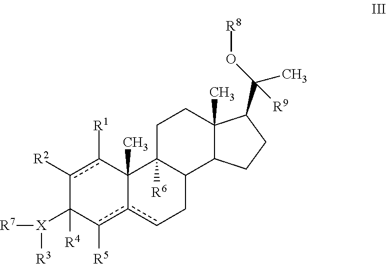

wherein X is O, N or S;

R¹, R², R⁵ and R⁶ are independently hydrogen, alkyl, halogen, hydroxylcycloalkyl, cycloalkenyl, alkenyl, alkynyl, aryl, alkylaryl, arylalkyl, heterocyclic, heteroaryl, amino, thiol, alkoxy, sulfide, nitro, cyano, azide, sulfonyl, acyl, carboxyl, an ester, an amide, carbamate, carbonate, an amino acid residue or a carbohydrate;

R³ is hydrogen, optionally substituted acyl, a residue of an amino acid a carbohydrate; —OR¹¹; —NR¹¹R¹² or R³ is absent;

R⁴ is hydrogen or alkyl; or R⁴ together with R⁷ form a doubleb bond;

R⁸ is hydrogen, optionally substituted acyl, a residue of an amino acid or a carbohydrate;

R⁹ is hydrogen or alkyl;

R¹¹ is the residue of an amino acid, a carbohydrate or optionally substituted acyl;

R¹² is hydrogen or alkyl; and the dotted lines indicate the presence of either a single bond or a double bond, wherein the valences of a single bond are completed by hydrogens, provided that if the dotted line between C4 and C5 or between C5 and C6 represents a double bond then the other dotted line between C4 and C5 or between C5 and C6 represents a single bond; and with the proviso that neither XR³R⁷ nor R⁸ represent an ester of aspartic acid, glutamic acid, gama amino butyric acid or a-2-(hydroxyethylamino)-propionic acid.

In one embodiment, R¹, R², R⁵ and R⁶ are independently hydrogen, alkyl, halogen or hydroxyl.

In another embodiment, R¹, R², R⁵ and R⁶ are independently methyl, ethyl or propyl.

In still another embodiment, R¹, R², R⁵ and R⁶ are independently thiomethyl, hydroxymethyl or cyano.

In another embodiment, R¹, R², R⁵ and R⁶ are independently vinyl or ethynyl.

In still another embodiment, R¹, R², R⁵ and R⁶ are independently fluoro, bromo, chloro or iodo.

In still another embodiment, R⁴ and R⁹ are independently hydrogen or methyl.

In some embodiments, X is O. In other embodiments, X is N. In certain embodiments in which X is O, R³ and R⁴ come together to form a double bond. In certain embodiments, one of R³ and R⁸ is a residue of an amino acid. In particular embodiments, the amino acid is a naturally occurring amino acid. In certain embodiments, R³ is a residue of an amino acid. In certain other embodiments, R⁸ is a residue of an amino acid. In yet further embodiments, both R³ and R⁸ are residues of an amino acid.

In one embodiment of Formula III, X is O; R³ is the residue of an amino acid; and R⁷ is absent.

In another embodiment of Formula III, X is N; R⁴ together with R⁷ form a double bond; R³ is OR¹¹; and R¹¹ is the residue of an amino acid.

In another embodiment of Formula III, X is N; R⁴ together with R⁷ form a double bond; R³ is —NR¹¹R¹²; R¹¹ is the residue of an amino acid; and R¹² is hydrogen.

In another embodiment of Formula III, R⁸ is the residue of a naturally occurring amino acid; R⁹ is hydrogen; X is O; R⁴ together with R⁷ form a double bond; and R³ is absent.

In another embodiment of Formula III, R⁸ is a carbohydrate; R⁹ is hydrogen; X is O; R⁴ together with R⁷ form a double bond; and R³ is absent.

In another embodiment of Formula III, R⁸ is the residue of a naturally occurring amino acid; R⁹ is hydrogen; X is O; R³ and R⁴ are hydrogen; and R⁷ is absent.

In another embodiment of Formula III, R⁶ is alkyl or fluoro. In yet another embodiment of Formula III, R¹, R² and R⁵ are independently hydrogen or alkyl. In another embodiment, R¹ and R² are hydroxyl. In still another embodiment, R¹ and R² are independently hydroxyl or halogen. In another embodiment of Formula III, R¹ is alkyl; and R² and R⁵ are hydrogen. In another embodiment, of Formula III, R² is alkyl; and R¹ and R⁵ are hydrogen. In still another embodiment, of Formula III, R⁵ is alkyl; and R¹ and R² are hydrogen.

In another embodiment of Formula III, R⁸ is the residue of a naturally occurring amino acid; R⁹ is hydrogen; X is O; R⁴ together with R⁷ form a double bond; R³ is absent; and R¹, R², R⁵ and R⁶ are hydrogen.

In another embodiment of Formula III, R⁸ is the residue of a naturally occurring amino acid; R⁹ is alkyl; X is O; R⁴ together with R⁷ form a double bond; R³ is absent; and R¹, R², R⁵ and R⁶ are hydrogen.

In one embodiment of Formula III, the dotted line between C4 and C5 represents a single bond and the dotted line between C5 and C6 represents a single bond.

In another embodiment of Formula III, the dotted line between C4 and C5 represents a single bond and the dotted line between C5 and C6 represents a double bond.

In another embodiment of Formula III, the dotted line between C4 and C5 represents a double bond and the dotted line between C5 and C6 represents a single bond.

In still another embodiment of Formula III, the dotted line between C1 and C2 represents a single bond. In still another embodiment, the dotted line between C1 and C2 represents a double bond.

In another embodiment of Formula III, —XR³R⁷ is in the alpha configuration. In still another embodiment, —XR³R⁷ is in the beta configuration.

In one embodiment of Formula III, the dotted lines between C4 and C5 and between C5 and C6 represent a single bond, and the hydrogen at the C5 bridgehead carbon is in the alpha configuration. In another embodiment, the dotted lines between C4 and C5 and between C5 and C6 represent a single bond, and the hydrogen at the C5 bridgehead carbon is in the beta configuration

In one embodiment of Formula III, R⁸ represents a naturally occurring ce-amino acid in the L-configuration. In another embodiment, R⁸ is a residue of L-valine. In another embodiment, R⁸ represents an amino acid residue with the D-configuration. In another embodiment, R⁸ represents a non-natural amino acid. In other embodiments, R⁸ represents the residue of a β γ or δ amino acid.

In one preferred embodiment of Formula III, R⁸ represents an ester of an amino acid. In another embodiment, R⁸ represents an ester of an amino acid residue where the ester bond is formed with a carboxylate group on the side chain of the amino acid.

In one preferred embodiment of Formula III, R³ represents an ester of an amino acid. In another embodiment, R³ represents an ester of an amino acid residue where the ester bond is formed with a carboxylate group on the side chain of the amino acid. In certain embodiments of Formula III, a residue of an amino acid is connected to the steroid ring system at the carboxyl group of the amino acid. In other embodiments, a residue of an amino acid is connected to the steroid at the amino acid side chain. For example, amino acids that contain side chains with functional groups that are capable of forming a bond with a hydroxy or a ketone group may be boded to the steroid ring by such a group. In other embodiments, the reactive groups on the amino acid side chains may displace leaving groups formed on the steroid moiety to form a covalent bond. Non-limiting examples of amino acids with reactive groups in the side chain include lysine, cysteine, serine, tyrosine, aspartic acid, arginine and the like.

The amino acid(s) in any of the embodiments of the invention described herein may be naturally occurring or synthetic amino acids and may be in the D or L stereoisomeric form or may exist as a D, L mixture. For example the 20 naturally occurring α-amino acids in the L-configuration are encompassed by the invention as well as β-amino acids in the D-configuration. Synthetic amino acids in either stereoisomeric form are also encompassed.

In another embodiment, the enantiomers of the compounds of Formula III are provided. In this embodiment, the stereochemical configuration of each asymmetric carbon is opposite that of the natural steroids and analogues of the natural steroids. For example, the configuration of C9, C10, C13 and C17 carbon atoms would be opposite to the configuration as drawn in the structure above.

In another embodiment a compound of Formula IV is provided:

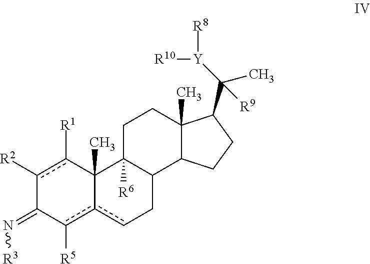

wherein Y is O, N or S;

R¹, R², R⁵ and R⁶ are independently hydrogen, alkyl, halogen, hydroxylcycloalkyl, cycloalkenyl, alkenyl, alkynyl, aryl, alkylaryl, arylalkyl, heterocyclic, heteroaryl, amino, thiol, alkoxy, sulfide, nitro, cyano, azide, sulfonyl, acyl, carboxyl, an ester, an amide, carbamate, carbonate, an amino acid residue or a carbohydrate;

R³ is an —OR¹¹, —NR¹¹R¹² or a carbohydrate;

R⁸ is hydrogen, optionally substituted acyl, a residue of an amino acid, a carbohydrate, —OR¹¹, —NR¹¹R¹² or R⁸ is absent;

R⁹ is hydrogen or alkyl; or R⁹ and R¹⁰ together form a double bond;

R¹⁰ is hydrogen or absent, or R¹⁰ together with R⁹ form a double bond;

R¹¹ is the residue of an amino acid, a carbohydrate or an optionally substituted cstcr;

R¹² is hydrogen or alkyl; and the dotted lines indicate the presence of either a single bond or a double bond, wherein the valences of a single bond are completed by hydrogens, provided that if the dotted line between C4 and C5 or between C5 and C6 represents a double bond then the other dotted line between C4 and C5 or between C5 and C6 represents a single bond; and with the proviso that YR⁸R¹⁰ does not represent an ester of aspartic acid, glutamic acid, gama amino butyric acid or a-2-(hydroxyethylamino)-propionic acid; and with the proviso that when Y is N, R⁸ does not represent aspartic acid, glutamic acid, gama amino butyric acid or a-2-(hydroxyethylamino)-propionic acid.

In one embodiment, R¹, R², R⁵ and R⁶ are independently hydrogen, alkyl, halogen or hydroxyl.

In another embodiment, R¹, R², R⁵ and R⁶ are independently methyl, ethyl or propyl.

In still another embodiment, R¹, R², R⁵ and R⁶ are independently thiomethyl, hydroxymethyl or cyano.

In another embodiment, R¹, R², R⁵ and R⁶ are independently vinyl or ethynyl.

In still another embodiment, R¹, R², R⁵ and R⁶ are independently fluoro, bromo, chloro or iodo.

In still another embodiment, R⁹ is hydrogen or methyl.

In some embodiments, Y is O. In other embodiments, Y is N. In certain embodiments in which Y is O, R⁸ and R¹⁰ come together to form a double bond. In certain embodiments, one of R³ and R⁸ is a residue of an amino acid. In particular embodiments, the amino acid is a naturally occurring amino acid. In certain embodiments, R³ is a residue of an amino acid. In certain other embodiments, R⁸ is a residue of an amino acid. In yet further embodiments, both R³ and R⁸ are residues of an amino acid.

In one embodiment of Formula IV, Y is O; R⁸ is the residue of an amino acid; and R¹⁰ is absent.

In another embodiment of Formula IV, Y is N; R¹⁰ together with R⁹ form a double bond; R⁸ is OR¹¹; and R¹¹ is the residue of an amino acid.

In another embodiment of Formula IV, Y is N; R¹⁰ together with R⁹ form a double bond; R⁸ is —NR¹¹R¹²; R¹¹ is the residue of an amino acid; and R¹² is hydrogen.

In another embodiment of Formula IV, R³ is —OR¹¹ and R¹¹ is the residue of a naturally occurring amino acid; Y is O; R¹⁰ together with R⁹ form a double bond; and R⁸ is absent.

In another embodiment of Formula IV, R³ is —NR¹¹R¹²; R¹¹ is the residue of a naturally occurring amino acid; R¹² is hydrogen; Y is O; R¹⁰ together with R⁹ form a double bond; and R⁸ is absent.

In another embodiment of Formula IV, R³ is a carbohydrate; Y is O; R¹⁰ together with R⁹ form a double bond; and R⁸ is absent.

In another embodiment of Formula IV, R³ is —OR¹¹ and R¹¹ is the residue of a naturally occurring amino acid; Y is O; R⁸ and R⁹ are hydrogen; and R¹⁰ is absent.

In another embodiment of Formula IV, R³ is —NR¹¹R¹²; R¹¹ is the residue of a naturally occurring amino acid; R¹² is hydrogen; Y is O; R⁸ and R⁹ are hydrogen; and R¹⁰ is absent.

In another embodiment of Formula IV, R⁶ is alkyl or fluoro. In yet another embodiment of Formula IV, R¹, R² and R⁵ are independently hydrogen or alkyl. In another embodiment, R¹ and R² are hydroxyl. In still another embodiment, R¹ and R² are independently hydroxyl or halogen. In another embodiment of Formula IV, R¹ is alkyl; and R² and R⁵ are hydrogen. In another embodiment, of Formula IV, R² is alkyl; and R¹ and R⁵ are hydrogen. In still another embodiment, of Formula IV, R⁵ is alkyl; and R¹ and R² are hydrogen.

In another embodiment of Formula IV, R³ is —OR¹¹ and R¹¹ is the residue of a naturally occurring amino acid; Y is O; R¹⁰ together with R⁹ form a double bond; R⁸ is absent; and R¹, R², R⁵ and R⁶ are hydrogen.

In another embodiment of Formula IV, R³ is —NR¹¹R¹²; R¹¹ is the residue of a naturally occurring amino acid; R¹² is hydrogen; Y is O; R¹⁰ together with R⁹ form a double bond; R⁸ is absent; and R¹, R², R⁵ and R⁶ are hydrogen.

In one embodiment of Formula IV, the dotted line between C4 and C5 is a single bond and the dotted line between C5 and C6 is a single bond.

In another embodiment of Formula IV, the dotted line between C4 and C5 is a single bond and the dotted line between C5 and C6 is a double bond.

In another embodiment of Formula IV, the dotted line between C4 and C5 is a double bond and the dotted line between C5 and C6 is a single bond.

In still another embodiment of Formula IV, the dotted line between C1 and C2 represents a single bond. In still another embodiment, the dotted line between C1 and C2 represents a double bond.

In one embodiment of Formula IV, the dotted lines between C4 and C5 and between C5 and C6 represent a single bond, and the hydrogen at the C5 bridgehead carbon is in the alpha configuration. In another embodiment, the dotted lines between C4 and C5 and between C5 and C6 represent a single bond, and the hydrogen at the C5 bridgehead carbon is in the beta configuration

In one embodiment of Formula IV, R³ comprises a residue of a naturally occurring α-amino acid in the L-configuration. In another embodiment, R³ comprises a residue of L-valine. In another embodiment, R³ comprises an amino acid residue with the D-configuration. In another embodiment, R³ comprises a non-natural amino acid. In other embodiments, R³ comprises the residue of a β γ or δ amino acid.

In one preferred embodiment of Formula IV, R³ represents an ester of an amino acid. In another embodiment, R³ represents an ester of an amino acid residue where the ester bond is formed with a carboxylate group on the side chain of the amino acid. In certain embodiments of Formula IV, a residue of an amino acid is connected to the steroid ring system at the carboxyl group of the amino acid. In other embodiments, a residue of an amino acid is connected to the steroid at the amino acid side chain. For example, amino acids that contain side chains with functional groups that are capable of forming a bond with a hydroxy or a ketone group may be boded to the steroid ring by such a group. In other embodiments, the reactive groups on the amino acid side chains may displace leaving groups formed on the steroid moiety to form a covalent bond. Non-limiting examples of amino acids with reactive groups in the side chain include lysine, cysteine, serine, tyrosine, aspartic acid, arginine and the like.

The amino acid(s) in any of the embodiments of the invention described herein may be naturally occurring or synthetic amino acids and may be in the D or L stereoisomeric form or may exist as a D, L mixture. For example the 20 naturally occurring α-amino acids in the L-configuration are encompassed by the invention as well as β-amino acids in the D-configuration. Synthetic amino acids in either stereoisomeric form are also encompassed.

In another embodiment, the enantiomers of the compounds of Formula IV are provided. In this embodiment, the stereochemical configuration of each asymmetric carbon is opposite that of the natural steroids and analogues of the natural steroids. For example, the configuration of C9, C10, C13 and C17 carbon atoms would be opposite to the configuration as drawn in the structure above.

In another embodiment, a neuroprotective steroid of Formula V is provided:

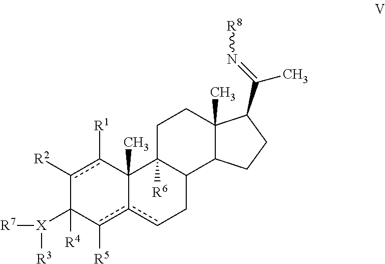

wherein X is O, N or S;

R¹, R², R⁵ and R⁶ are independently hydrogen, alkyl, halogen, hydroxylcycloalkyl, cycloalkenyl, alkenyl, alkynyl, aryl, alkylaryl, arylalkyl, heterocyclic, heteroaryl, amino, thiol, alkoxy, sulfide, nitro, cyano, azide, sulfonyl, acyl, carboxyl, an ester, an amide, carbamate, carbonate, an amino acid residue or a carbohydrate;

R³ is hydrogen, optionally substituted acyl, a residue of an amino acid a carbohydrate; —OR¹¹; —NR¹¹R¹² or R³ is absent;

R⁴ is hydrogen or alkyl; or R⁴ together with R⁷ form a double bond;

R⁸ is —OR¹¹, —NR¹¹R¹² or a carbohydrate;

R¹¹ is the residue of an amino acid, a carbohydrate or an optionally substituted ester;

R¹² is hydrogen or alkyl; and the dotted lines indicate the presence of either a single bond or a double bond, wherein the valences of a single bond are completed by hydrogens, provided that if the dotted line between C4 and C5 or between C5 and C6 represents a double bond then the other dotted line between C4 and C5 or between C5 and C6 represents a single bond; and with the proviso that XR³R⁷ does not represent an ester of aspartic acid, glutamic acid, gama amino butyric acid or a-2-(hydroxyethylamino)-propionic acid; and with the proviso that R⁸ docs not represent aspartic acid, glutamic acid, gama amino butyric acid or a-2-(hydroxyethylamino)-propionic acid.

In one embodiment, R¹, R², R⁵ and R⁶ are independently hydrogen, alkyl, halogen or hydroxyl.

In another embodiment, R¹, R², R⁵ and R⁶ are independently methyl, ethyl or propyl.

In still another embodiment, R¹, R², R⁵ and R⁶ are independently thiomethyl, hydroxymethyl or cyano.

In another embodiment, R¹, R², R⁵ and R⁶ are independently vinyl or ethynyl.

In still another embodiment, R¹, R², R⁵ and R⁶ are independently fluoro, bromo, chloro or iodo.

In still another embodiment, R⁴ is hydrogen or methyl.

In some embodiments, X is O. In other embodiments, X is N. In certain embodiments in which X is O, R³ and R⁴ come together to form a double bond. In certain embodiments, one of R³ and R⁸ is a residue of an amino acid. In particular embodiments, the amino acid is a naturally occurring amino acid. In certain embodiments, R³ is a residue of an amino acid. In certain other embodiments, R⁸ is a residue of an amino acid. In yet further embodiments, both R³ and R⁸ are residues of an amino acid.

In one embodiment of Formula V, X is O; R³ is the residue of an amino acid; and R⁷ is absent.

In another embodiment of Formula V, X is N; R⁴ together with R⁷ form a double bond; R³ is OR¹¹; and R¹¹ is the residue of an amino acid.

In another embodiment of Formula V, X is N; R⁴ together with R⁷ form a double bond; R³ is —NR¹¹R¹²; R¹¹ is the residue of an amino acid; and R¹² is hydrogen.

In another embodiment of Formula V, R⁸ is —OR¹¹; R¹¹ is the residue of a naturally occurring amino acid; X is O; R⁴ together with R⁷ form a double bond; and R³ is absent.

In another embodiment of Formula V, R⁸ is —NR¹¹R¹²; R¹¹ is the residue of a naturally occurring amino acid; R¹² is hydrogen; X is O; R⁴ together with R⁷ form a double bond; and R³ is absent.

In another embodiment of Formula V, R⁸ is a carbohydrate; X is O; R⁴ together with R⁷ form a double bond; and R³ is absent.

In another embodiment of Formula V, R⁸ is —OR¹¹; R¹¹ is the residue of a naturally occurring amino acid; X is O; R³ and R⁴ are hydrogen; and R⁷ is absent.

In another embodiment of Formula V, R⁸ is —NR¹¹R¹²; R¹¹ is the residue of a naturally occurring amino acid; R¹² is hydrogen; X is O; R³ and R⁴ are hydrogen; and R⁷ is absent.

In another embodiment of Formula V, R⁶ is alkyl or fluoro. In yet another embodiment of Formula V, R¹, R² and R⁵ are independently hydrogen or alkyl. In another embodiment, R¹ and R² are hydroxyl. In still another embodiment, R¹ and R² are independently hydroxyl or halogen. In another embodiment of Formula V, R¹ is alkyl; and R² and R⁵ are hydrogen. In another embodiment, of Formula V, R² is alkyl; and R¹ and R⁵ are hydrogen. In still another embodiment, of Formula V, R⁵ is alkyl; and R¹ and R² are hydrogen.

In another embodiment of Formula V, R⁸ is —OR¹¹; R¹¹ is the residue of a naturally occurring amino acid; X is O; R⁴ together with R⁷ form a double bond; R³ is absent; and R¹, R², R⁵ and R⁶ are hydrogen.

In another embodiment of Formula V, R⁸ is —NR¹¹R¹²; R¹¹ is the residue of a naturally occurring amino acid; R¹² is hydrogen; X is O; R⁴ together with R⁷ form a double bond; R³ is absent; and R¹, R², R⁵ and R⁶ are hydrogen.

In another embodiment of Formula V, R⁸ is —OR¹¹; R¹¹ is the residue of a naturally occurring amino acid; X is O; R³ and R⁴ are hydrogen; R⁷ is absent; and R¹, R², R⁵ and R⁶ are hydrogen.

In another embodiment of Formula V, R⁸ is —NR¹¹R¹²; R¹¹ is the residue of a naturally occurring amino acid; R¹² is hydrogen; X is O; R³ and R⁴ are hydrogen; R⁷ is absent; and R¹, R², R⁵ and R⁶ are hydrogen; and R¹, R², R⁵ and R⁶ are hydrogen.

In one embodiment of Formula V, the dotted line between C4 and C5 is a single bond and the dotted line between C5 and C6 is a single bond.

In another embodiment of Formula V, the dotted line between C4 and C5 is a single bond and the dotted line between C5 and C6 is a double bond.

In another embodiment of Formula V, the dotted line between C4 and C5 is a double bond and the dotted line between C5 and C6 is a single bond.

In still another embodiment of Formula V, the dotted line between C1 and C2 represents a single bond. In still another embodiment, the dotted line between C1 and C2 represents a double bond.

In another embodiment of Formula V, —XR³R⁷ is in the alpha configuration. In still another embodiment, —XR³R⁷ is in the beta configuration.

In one embodiment of Formula V, the dotted lines between C4 and C5 and between C5 and C6 represent a single bond, the hydrogen at the C5 bridgehead carbon is in the alpha configuration. In another embodiment, the dotted lines between C4 and C5 and between C5 and C6 represent a single bond, the hydrogen at the C5 bridgehead carbon is in the beta configuration

In one embodiment of Formula V, R⁸ comprises a naturally occurring α-amino acid in the L-configuration. In another embodiment, R⁸ comprises a residue of L-valine. In another embodiment, R⁸ comprises an amino acid residue with the D-configuration. In another embodiment, R⁸ comprises a non-natural amino acid. In other embodiments, R⁸ comprises the residue of a β γ or δ amino acid.

In one preferred embodiment of Formula V, R³ represents an ester of an amino acid. In another embodiment, R³ represents an ester of an amino acid residue where the ester bond is formed with a carboxylate group on the side chain of the amino acid. In certain embodiments of Formula V, a residue of an amino acid is connected to the steroid ring system at the carboxyl group of the amino acid. In other embodiments, a residue of an amino acid is connected to the steroid at the amino acid side chain. For example, amino acids that contain side chains with functional groups that are capable of forming a bond with a hydroxy or a ketone group may be boded to the steroid ring by such a group. In other embodiments, the reactive groups on the amino acid side chains may displace leaving groups formed on the steroid moiety to form a covalent bond. Non-limiting examples of amino acids with reactive groups in the side chain include lysine, cysteine, serine, tyrosine, aspartic acid, arginine and the like.

The amino acid(s) in any of the embodiments of the invention described herein may be naturally occurring or synthetic amino acids and may be in the D or L stereoisomeric form or may exist as a D, L mixture. For example the 20 naturally occurring α-amino acids in the L-configuration are encompassed by the invention as well as β-amino acids in the D-configuration. Synthetic amino acids in either stereoisomeric form are also encompassed.

In another embodiment, the enantiomers of the compounds of Formula V are provided. In this embodiment, the stereochemical configuration of each asymmetric carbon is opposite that of the natural steroids and analogues of the natural steroids. For example, the configuration of C9, C10, C13 and C17 carbon atoms would be opposite to the configuration as drawn in the structure above.

In another embodiment of the invention, a progesterone analogue of Formula VI is provided:

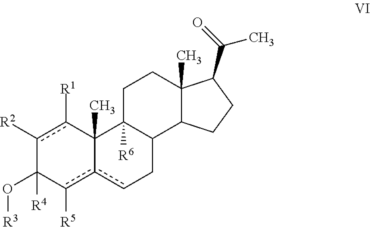

wherein R¹, R², R⁵ and R⁶ are independently hydrogen, alkyl, halogen, hydroxylcycloalkyl, cycloalkenyl, alkenyl, alkynyl, aryl, alkylaryl, arylalkyl, heterocyclic, heteroaryl, amino, thiol, alkoxy, sulfide, nitro, cyano, azide, sulfonyl, acyl, carboxyl, an ester, an amide, carbamate, carbonate, an amino acid residue or a carbohydrate;
R³ is hydrogen, optionally substituted acyl, a residue of an amino acid or a carbohydrate;
R⁴ is hydrogen or alkyl; and the dotted lines indicate the presence of either a single bond or a double bond, wherein the valences of a single bond are completed by hydrogens, provided that if the dotted line between C4 and C5 or between C5 and C6 represents a double bond then the other dotted line between C4 and C5 or between C5 and C6 represents a single bond; and with the proviso that neither R³ does not represent an ester of aspartic acid, glutamic acid, gama amino butyric acid or a-2-(h ydroxyethylamino)-propionic acid.

In another embodimcnt of Formula VI, R⁶ is alkyl or fluoro. In yet another embodimcnt of Formula VI, R¹, R² and R⁵ are independently hydrogen or alkyl. In another embodiment, R¹ and R² are hydroxyl. In still another embodiment, R¹ and R² are independently hydroxyl or halogen. In another embodiment of Formula VI, R¹ is alkyl; and R² and R⁵ are hydrogen. In another embodiment, of Formula VI, R² is alkyl; and R¹ and R⁵ are hydrogen. In still another embodiment, of Formula VI, R⁵ is alkyl; and R¹ and R² are hydrogen.

In one embodiment, R¹, R², R⁵ and R⁶ are independently hydrogen, alkyl, halogen or hydroxyl.

In another embodimcnt, R¹, R², R⁵ and R⁶ are independently methyl, ethyl or propyl.

In still another embodiment, R¹, R², R⁵ and R⁶ are independently thiomethyl, hydroxymethyl or cyano.

In another embodiment, R¹, R², R⁵ and R⁶ are independently vinyl or ethynyl.

In still another embodiment, R¹, R², R⁵ and R⁶ are independently fluoro, bromo, chloro or iodo.

In still another embodiment, R⁴ is hydrogen or methyl.

In another embodiment of Formula VI, R³ is the residue of a naturally occurring amino acid; and R¹, R², R⁴, R⁵ and R⁶ are hydrogen.

In another embodiment of Formula VI, R³ is the residue of a naturally occurring amino acid; R⁴ is alkyl; and R¹, R², R⁵ and R⁶ are hydrogen.

In one embodiment of Formula VI, R¹ is alkyl; and R², R⁴ and R⁵ are hydrogen.

In another embodiment of Formula VI, R¹ and R⁴ are alkyl; and R² and R⁵ are hydrogen.

In another embodiment of Formula VI, R³ is a carbohydrate; and R¹, R², R⁴, R⁵ and R⁶ are hydrogen.

In one embodiment of Formula VI, the dotted line between C4 and C5 represents a single bond and the dotted line between C5 and C6 represents a single bond.

In another embodiment of Formula VI, the dotted line between C4 and C5 represents a single bond and the dotted line between C5 and C6 represents a double bond.

In another embodiment of Formula VI, the dotted line between C4 and C5 represents a double bond and the dotted line between C5 and C6 represents a single bond.

In still another embodiment of Formula VI, the dotted line between C1 and C2 represents a single bond. In still another embodiment, the dotted line between C1 and C2 represents a double bond.

In another embodiment of Formula VI, OR³ is in the alpha configuration. In still another embodiment, OR³ is in the beta configuration.

In one embodiment of Formula VI, the dotted lines between C4 and C5 and between C5 and C6 represent a single bond, and the hydrogen at the C5 bridgehead carbon is in the alpha configuration. In another embodiment, the dotted lines between C4 and C5 and between C5 and C6 represent a single bond, and the hydrogen at the C5 bridgehead carbon is in the beta configuration

In one embodiment of Formula VI, R³ represents a naturally occurring α-amino acid in the L-configuration. In another embodiment, R³ is a residue of L-valine. In another embodiment, R³ represents an amino acid residue with the D-configuration. In another embodiment, R³ represents a non-natural amino acid. In other embodiments, R³ represents the residue of a β γ or δ amino acid.

In one preferred embodiment of Formula VI, R³ represents an ester of an amino acid. In another embodiment, R³ represents an ester of an amino acid residue where the ester bond is formed with a carboxylate group on the side chain of the amino acid. In certain embodiments of Formula VI, a residue of an amino acid is connected to the steroid ring system at the carboxyl group of the amino acid. In other embodiments, a residue of an amino acid is connected to the steroid at the amino acid side chain. For example, amino acids that contain side chains with functional groups that are capable of forming a bond with a hydroxy or a ketone group may be boded to the steroid ring by such a group. In other embodiments, the reactive groups on the amino acid side chains may displace leaving groups formed on the steroid moicty to form a covalent bond. Non-limiting examples of amino acids with reactive groups in the side chain include lysine, cysteine, mine, tyrosine, aspartic acid, arginine and the like.

The amino acid(s) in any of the embodiments of the invention described herein may be naturally occurring or synthetic amino acids and may be in the D or L stereoisomeric form or may exist as a D, L mixture. For example the 20 naturally occurring α-amino acids in the L-configuration are encompassed by the invention as well as β-amino acids in the D-configuration. Synthetic amino acids in either stereoisomeric form are also encompassed.

In another embodiment, the enantiomers of the compounds of Formula VI are provided. In this embodiment, the stcreochemical configuration of each asymmetric carbon is opposite that of the natural steroids and analogues of the natural steroids. For example, the configuration of C9, C10C, C13 and C17 carbon atoms would be opposite to the configuration as drawn in the structure above.

In still another embodiment, a progesterone analogue of Formula VII is provided:

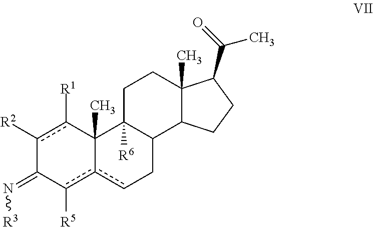

wherein, R¹, R², R⁵ and R⁶ are independently hydrogen, alkyl, halogen, hydroxyl cycloalkyl, cycloalkenyl, alkenyl, alkynyl, aryl, alkylaryl, arylalkyl, heterocyclic, heteroaryl, amino, thiol, alkoxy, sulfide, nitro, cyano, azide, sulfonyl, acyl, carboxyl, an ester, an amide, carbamate, carbonate, an amino acid residue or a carbohydrate;

R³ is —OR¹¹, —NR¹¹R¹² or a carbohydrate;

R¹¹ is the residue of an amino acid, a carbohydrate or an optionally substituted ester;

R¹² is hydrogen or alkyl; and the dotted lines indicate the presence of either a single bond or a double bond, wherein the valences of a single bond are completed by hydrogens, provided that if the dotted line between C4 and C5 or between C5 and C6 represents a double bond then the other dotted line between C4 and C5 or between C5 and C6 represents a single bond.

In one embodiment, R¹, R², R⁵ and R⁶ are independently hydrogen, alkyl, halogen or hydroxyl.

In another embodiment, R¹, R², R⁵ and R⁶ are independently methyl, ethyl or propyl.

In still another embodiment, R¹, R², R⁵ and R⁶ are independently thiomethyl, hydroxymethyl or cyano.

In another embodiment, R¹, R², R⁵ and R⁶ are independently vinyl or ethynyl.

In still another embodiment, R¹, R², R⁵ and R⁶ are independently fluoro, bromo, chloro or iodo.

In one embodiment of Formula VII, R⁶ is alkyl or fluoro. In yet another embodiment of Formula VII, R¹, R² and R⁵ are independently hydrogen or alkyl. In another embodiment, R¹ and R² are hydroxyl. In still another embodiment, R¹ and R² are independently hydroxyl or halogen. In another embodiment of Formula VII, R¹ is alkyl; and R² and R⁵ are hydrogen. In another embodiment, of Formula VII, R² is alkyl; and R¹ and R⁵ are hydrogen. In still another embodiment, of Formula VII, R⁵ is alkyl; and R¹ and R² are hydrogen.

In another embodiment of Formula VII, R³ is —OR¹¹ and R¹¹ is the residue of a naturally occurring amino acid; and R¹, R², R⁵ and R⁶ are hydrogen.

In another embodiment of Formula VII, R³ is —NR¹¹R¹²; R¹¹ is the residue of a naturally occurring amino acid; R¹² is hydrogen; and R¹, R², R⁵ and R⁶ are hydrogen.

In another embodiment of Formula VII, R³ is a carbohydrate; and R¹, R², R⁵ and R⁶ are hydrogen.

In one embodiment of Formula VII, the dotted line between C4 and C5 represents a single bond and the dotted line between C5 and C6 represents a single bond.

In another embodiment of Formula VII, the dotted line between C4 and C5 represents a single bond and the dotted line between C5 and C6 represents a double bond.

In another embodiment of Formula VII, the dotted line between C4 and C5 represents a double bond and the dotted line between C5 and C6 represents a single bond.

In still another embodiment of Formula VII, the dotted line between C1 and C2 represents a single bond. In still another embodiment, the dotted line between C1 and C2 represents a double bond.

In one embodiment of Formula VII, the dotted lines between C4 and C5 and between C5 and C6 represent a single bond, and the hydrogen at the C5 bridgehead carbon is in the alpha configuration. In another embodiment, the dotted lines between C4 and C5 and between C5 and C6 represent a single bond, and the hydrogen at the C5 bridgehead carbon is in the beta configuration

In one embodiment of Formula VII, R³ comprises a naturally occurring α-amino acid in the L-configuration. In another embodiment, R³ comprises a residue of L-valine. In another embodiment, R³ comprises an amino acid residue with the D-configuration. In another embodiment, R³ comprises a non-natural amino acid. In other embodiments, R³ comprises the residue of a β γ or δ amino acid.

In one preferred embodiment of Formula V, R³ represents an ester of an amino acid. In another embodiment, R³ represents an ester of an amino acid residue where the ester bond is formed with a carboxylate group on the side chain of the amino acid. In certain embodiments of Formula V, a residue of an amino acid is connected to the steroid ring system at the carboxyl group of the amino acid. In other embodiments, a residue of an amino acid is connected to the steroid at the amino acid side chain. For example, amino acids that contain side chains with functional groups that are capable of forming a bond with a hydroxy or a ketone group may be boded to the steroid ring by such a group. In othcr embodiments, the reactive groups on the amino acid side chains may displace leaving groups formed on the steroid moiety to form a covalent bond. Non-limiting examples of amino acids with reactive groups in the side chain include lysine, cysteine, serine, tyrosine, aspartic acid, arginine and the like.

The amino acid(s) in any of the embodiments of the invention described herein may be naturally occurring or synthetic amino acids and may be in the D or L stereoisomeric form or may exist as a D, L mixture. For example the 20 naturally occurring α-amino acids in the L-configuration are encompassed by the invention as well as β-amino acids in the D-configuration. Synthetic amino acids in either stereoisomeric form are also encompassed.

In another embodiment, the enantiomers of the compounds of Formula VII are provided. In this embodiment, the stereochcmical configuration of each asymmetric carbon is opposite that of the natural steroids and analogues of the natural steroids. For example, the configuration of C9, C10, C13 and C17 carbon atoms would be opposite to the configuration as drawn in the structure above.

In another embodiment of the invention, a progesterone analogue of Formula VIII is provided:

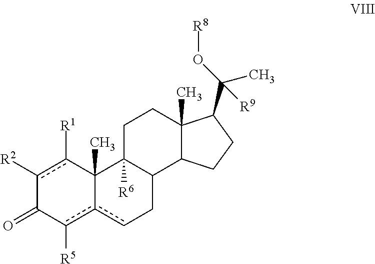

R¹, R², R⁵ and R⁶ are independently hydrogen, alkyl, halogen, hydroxylcycloalkyl, cycloalkenyl, alkenyl, alkynyl, aryl, alkyl aryl, arylalkyl, heterocyclic, heteroaryl, amino, thiol, alkoxy, sulfide, nitro, cyano, azide, sulfonyl, acyl, carboxyl, an ester, an amide, carbamate, carbonate, an amino acid residue or a carbohydrate;

R⁸ is hydrogen, optionally substituted acyl, a residue of an amino acid or a carbohydrate;

R⁹ is hydrogen or alkyl; and the dotted lines indicate the presence of either a single bond or a double bond, wherein the valences of a single bond are completed by hydrogens, provided that if the dotted line between C4 and C5 or between C5 and C6 represents a double bond then the other dotted line between C4 and C5 or between C5 and C6 represents a single bond; and with the proviso that R⁸ does not represent an ester of aspartic acid, glutamic acid, gama amino butyric acid or a-2-(hydroxyethylamino)-propionic acid; and with the proviso that when Y is N, R⁸ does not represent aspartic acid, glutamic acid, gama amino butyric acid or a-2-(hydroxyethylamino)-propionic acid.

In one embodiment, R¹, R², R⁵ and R⁶ are independently hydrogen, alkyl, halogen or hydroxyl.

In another embodiment, R¹, R², R⁵ and R⁶ are independently methyl, ethyl or propyl.

In still another embodiment, R¹, R², R⁵ and R⁶ are independently thiomethyl, hydroxymethyl or cyano.

In another embodiment, R¹, R², R⁵ and R⁶ are independently vinyl or ethynyl.

In still another embodiment, R¹, R², R⁵ and R⁶ are independently fluoro, bromo, chloro or iodo.

In still another embodiment, R⁹ is hydrogen or methyl.

In another embodiment of Formula VIII, R⁶ is alkyl or fluoro. In yet another embodiment of Formula VIII, R¹, R² and R⁵ are independently hydrogen or alkyl. In another embodiment, R¹ and R² are hydroxyl. In still another embodiment, R¹ and R² are independently hydroxyl or halogen. In another embodiment of Formula VIII, R¹ is alkyl; and R² and R⁵ are hydrogen. In another embodiment, of Formula VIII, R² is alkyl; and R¹ and R⁵ are hydrogen. In still another embodiment, of Formula VIII, R⁵ is alkyl; and R¹ and R² are hydrogen.

In another embodiment of Formula VIII, R⁸ is the residue of a naturally occurring amino acid; and R¹, R², R⁵, R⁶ and R⁹ are hydrogen.

In another embodiment of Formula VIII, R⁸ is the residue of a naturally occurring amino acid; R⁹ is alkyl; and R¹, R², R⁵ and R⁶ are hydrogen.

In one embodiment of Formula VIII, R¹ is alkyl; and R², R⁵ and R⁹ are hydrogen.

In another embodiment of Formula VIII, R¹ and R⁹ are alkyl; and R² and R⁵ are hydrogen.

In another embodiment of Formula VIII, R⁸ is a carbohydrate; and R¹, R², R⁵, R⁶ and R⁹ are hydrogen.

In one embodiment of Formula VIII, the dotted line between C4 and C5 represents a single bond and the dotted line between C5 and C6 represents a single bond.

In another embodiment of Formula VIII, the dotted line between C4 and C5 represents a single bond and the dotted line between C5 and C6 represents a double bond.

In another embodiment of Formula VIII, the dotted line between C4 and C5 represents a double bond and the dotted line between C5 and C6 represents a single bond.

In still another embodiment of Formula VIII, the dotted line between C1 and C2 represents a single bond. In still another embodiment, the dotted line between C1 and C2 represents a double bond.

In one embodiment of Formula VIII, the dotted lines between C4 and C5 and between C5 and C6 represent a single bond, and the hydrogen at the C5 bridgehead carbon is in the alpha configuration. In another embodiment, the dotted lines between C4 and C5 and between C5 and C6 represent a single bond, and the hydrogen at the C5 bridgehead carbon is in the beta configuration

In one embodiment of Formula VIII, R⁸ is the residue of a naturally occurring α-amino acid in the L-configuration. In another embodiment, R⁸ is a residue of L-valine. In another embodiment, R⁸ is an amino acid residue with the D-configuration. In another embodiment, R⁸ represents a residue of a non-natural amino acid. In other embodiments, R⁸ represents the residue of a β γ or δ amino acid.

In one preferred embodiment of Formula VIII, R⁸ represents an ester of an amino acid. In another embodiment, R⁸ represents an ester of an amino acid residue where the ester bond is formed with a carboxylate group on the side chain of the amino acid.

In another embodiment, the enantiomers of the compounds of Formula VIII are provided. In this embodiment, the stereochemical configuration of each asymmetric carbon is opposite that of the natural steroids and analogues of the natural steroids. For example, the configuration of C9, C10, C13 and C17 carbon atoms would be opposite to the configuration as drawn in the structure above.

In still another embodiment of the invention, a neuroprotective steroid of Formula IX is provided:

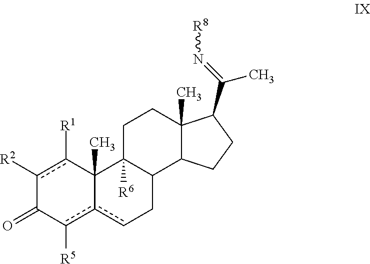

wherein R¹, R², R⁵ and R⁶ are independently hydrogen, alkyl, halogen, hydroxylcycloalkyl, cycloalkenyl, alkenyl, alkynyl, aryl, alkylaryl, arylalkyl, heterocyclic, heteroaryl, amino, thiol, alkoxy, sulfide, nitro, cyano, azide, sulfonyl, acyl, carboxyl, an ester, an amide, carbamate, carbonate, an amino acid residue or a carbohydrate;

R⁸ is —OR¹¹, —NR¹¹R¹² or a carbohydrate;

R¹¹ is the residue of an amino acid, a carbohydrate or an optionally substituted ester;

R¹² is hydrogen or alkyl; and the dotted lines indicate the presence of either a single bond or a double bond, wherein the valences of a single bond are completed by hydrogens, provided that if the dotted line between C4 and C5 or between C5 and C6 represents a double bond then the other dotted line between C4 and C5 or between C5 and C6 represents a single bond; and with the proviso that R⁸ does not represent aspartic acid, glutamic acid, gama amino butyric acid or a-2-(hydroxyethylamino)-propionic acid.

In one embodiment, R¹, R², R⁵ and R⁶ are independently hydrogen, alkyl, halogen or hydroxyl.

In another embodiment, R¹, R², R⁵ and R⁶ are independently methyl, ethyl or propyl.

In still another embodiment, R¹, R², R⁵ and R⁶ are independently thiomethyl, hydroxymethyl or cyano.

In another embodiment, R¹, R², R⁵ and R⁶ are independently vinyl or ethynyl.

In still another embodiment, R¹, R², R⁵ and R⁶ are independently fluoro, bromo, chloro or iodo.

In one embodiment of Formula IX, R⁶ is alkyl or fluoro. In yet another embodiment of Formula IX, R¹, R² and R⁵ are independently hydrogen or alkyl. In another embodiment, R¹ and R² are hydroxyl. In still another embodiment, R¹ and R² are independently hydroxyl or halogen. In another embodiment of Formula IX, R¹ is alkyl; and R² and R⁵ are hydrogen. In another embodiment, of Formula IX, R² is alkyl; and R¹ and R⁵ are hydrogen. In still another embodiment, of Formula IX, R⁵ is alkyl; and R¹ and R² are hydrogen.

In another embodiment of Formula IX, R³ is —OR¹¹ and R¹¹ is the residue of a naturally occurring amino acid; and R¹, R², R⁵ and R⁶ are hydrogen.

In another embodiment of Formula IX, R⁸ is —NR¹¹R¹²; R¹¹ is the residue of a naturally occurring amino acid; R¹² is hydrogen; and R¹, R², R⁵ and R⁶ are hydrogen.

In another embodiment of Formula IX, R⁸ is a carbohydrate; and R¹, R², R⁵ and R⁶ are hydrogen.

In one embodiment of Formula IX, the dotted line between C4 and C5 represents a single bond and the dotted line between C5 and C6 represents a single bond.

In another embodiment of Formula IX, the dotted line between C4 and C5 represents a single bond and the dotted line between C5 and C6 represents a double bond.

In another embodiment of Formula IX, the dotted line between C4 and C5 represents a double bond and the dotted line between C5 and C6 represents a single bond.

In still another embodiment of Formula IX, the dotted line between C1 and C2 represents a single bond. In still another embodiment, the dotted line between C1 and C2 represents a double bond.

In one embodiment of Formula IX, the dotted lines between C4 and C5 and between C5 and C6 represent a single bond, and the hydrogen at the C5 bridgehead carbon is in the alpha configuration. In another embodiment, the dotted lines between C4 and C5 and between C5 and C6 represent a single bond, and the hydrogen at the C5 bridgehead carbon is in the beta configuration

In one embodiment of Formula IX, R⁸ comprises a naturally occurring α-amino acid in the L-configuration. In another embodiment, R⁸ comprises a residue of L-valinc. In another embodiment, R⁸ comprises an amino acid residue with the D-configuration. In another embodiment, R⁸ comprises a non-natural amino acid. In other embodiments, R⁸ comprises the residue of a β γ or δ amino acid.

In another embodiment, the enantiomers of the compounds of Formula IX are provided. In this embodiment, the stereochemical configuration of each asymmetric carbon is opposite that of the natural steroids and analogues of the natural steroids. For example, the configuration of C9, C10, C13 and C17 carbon atoms would bc opposite to the configuration as drawn in the structure above.

In still another embodiment of the invention, a neuroprotective steroid of Formula X is provided:

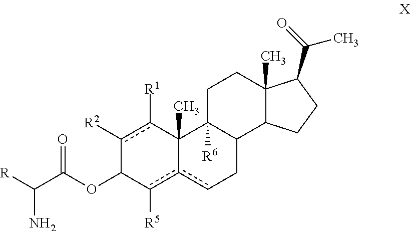

wherein R is the side chain of a naturally occurring amino acid; and R¹, R², R⁵ and R⁶ are independently hydrogen, alkyl, halogen, hydroxylcycloalkyl, cycloalkenyl, alkenyl, alkynyl, aryl, alkylaryl, arylalkyl, heterocyclic, heteroaryl, amino, thiol, alkoxy, sulfide, nitro, cyano, azide, sulfonyl, acyl, carboxyl, an ester, an amide, carbamate, carbonate, an amino acid residue or a carbohydrate; and with the proviso that R does not represent the side chain of aspartic acid, glutamic acid, gama amino butyric acid or a-2-(hydroxyethylamino)-propionic acid.

In one embodiment, R¹, R², R⁵ and R⁶ are independently hydrogen, alkyl, halogen or hydroxyl.

In another embodiment, R¹, R², R⁵ and R⁶ are independently methyl, ethyl or propyl.

In still another embodiment, R¹, R², R⁵ and R⁶ are independently thiomethyl, hydroxymethyl or cyano.

In another embodiment, R¹, R², R⁵ and R⁶ are independently vinyl or ethynyl.

In still another embodiment, R¹, R², R⁵ and R⁶ are independently fluoro, bromo, chloro or iodo.

In one embodiment of Formula X, R⁶ is alkyl or fluoro. In yet another embodiment of Formula X, R¹, R² and R⁵ are independently hydrogen or alkyl. In another embodiment, R¹ and R² are hydroxyl. In still another embodiment, R¹ and R² are independently hydroxyl or halogen. In another embodiment of Formula X, R¹, R², R⁵ and R⁶ are hydrogen. In one embodiment of Formula X, R¹ is alkyl; and R², and R⁵ are hydrogen. In another embodiment, of Formula X, R² is alkyl; and R¹ and R⁵ are hydrogen. In still another embodiment, of Formula X, R⁵ is alkyl; and R¹ and R² are hydrogen.

In one embodiment of Formula X, the dotted line between C4 and C5 represents a single bond and the dotted line between C5 and C6 represents a single bond.

In another embodiment of Formula X, the dotted line between C4 and C5 represents a single bond and the dotted line between C5 and C6 represents a double bond.

In another embodiment of Formula X, the dotted line between C4 and C5 represents a double bond and the dotted line between C5 and C6 represents a single bond.

In still another embodiment of Formula X, the dotted line between C1 and C2 represents a single bond. In still another embodiment, the dotted line between C1 and C2 represents a double bond.

In one embodiment of Formula X, the dotted lines between C4 and C5 and between C5 and C6 represent a single bond, and the hydrogen at the C5 bridgehead carbon is in the alpha configuration. In another embodiment, and the dotted lines between C4 and C5 and between C5 and C6 represent a single bond, the hydrogen at the C5 bridgehead carbon is in the beta configuration

In one embodiment of FOrmula X, R comprises the side chain of a naturally occurring α-amino acid in the L-configuration. In another embodiment, R comprises a residue of L-alanine, L-leucine, L-isoleucine, L-proline, or L-valine. In another embodiment, R comprises an amino acid residue with the D-configuration.

In another embodiment, the enantiomers of the compounds of Formula X are provided. In this embodiment, the stereochemical configuration of each asymmetric carbon is opposite that of the natural steroids and analogues of the natural steroids. For example, the configuration of C9, C10, C13 and C17 carbon atoms would be opposite to the configuration as drawn in the structure above.

In still another embodiment of the invention, a neuroprotective steroid of Formula XI is provided:

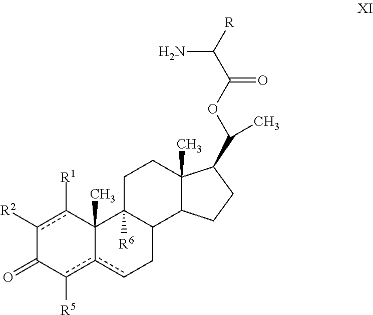

where R is the side chain of a naturally occurring amino acid; and R¹, R², R⁵ and R⁶ are independently hydrogen, alkyl, halogen, hydroxylcycloalkyl, cycloalkenyl, alkenyl, alkynyl, aryl, alkylaryl, arylalkyl, heterocyclic, heteroaryl, amino, thiol, alkoxy, sulfide, nitro, cyano, azide, sulfonyl, acyl, carboxyl, an ester, an amide, carbamate, carbonate, an amino acid residue or a carbohydrate; and with the proviso that R does not represent the side chain of aspartic acid, glutamic acid, gama amino butyric acid or a-2-(hydroxyethylamino)-propionic acid.

In one embodiment, R¹, R², R⁵ and R⁶ are independently hydrogen, alkyl, halogen or hydroxyl.

In another embodiment, R¹, R², R⁵ and R⁶ are independently methyl, ethyl or propyl.

In still another embodiment, R¹, R², R⁵ and R⁶ are independently thiomethyl, hydroxymethyl or cyano.

In another embodiment, R¹, R², R⁵ and R⁶ are independently vinyl or ethynyl.

In still another embodiment, R¹, R², R⁵ and R⁶ are independently fluoro, bromo, chloro or iodo.

In one embodiment of Formula X, R⁶ is alkyl or fluoro. In yet another embodiment of Formula X, R¹, R² and R⁵ are independently hydrogen or alkyl. In another embodiment, R¹ and R² are hydroxyl. In still another embodiment, R¹ and R² are independently hydroxyl or halogen. In another embodiment of Formula X, R¹, R², R⁵ and R⁶ are hydrogen. In one embodiment of Formula X, R¹ is alkyl; and R², and R⁵ are hydrogen. In another embodiment, of Formula X, R² is alkyl; and R¹ and R⁵ are hydrogen. In still another embodiment, of Formula X, R⁵ is alkyl; and R¹ and R² are hydrogen.

In one embodiment of Formula X, the dotted line between C4 and C5 represents a single bond and the dotted line between C5 and C6 represents a single bond.

In another embodiment of Formula X, the dotted line between C4 and C5 represents a single bond and the dotted line between C5 and C6 represents a double bond.

In another embodiment of Formula X, the dotted line between C4 and C5 represents a double bond and the dotted line between C5 and C6 represents a single bond.

In still another embodiment of Formula X, the dotted line between C1 and C2 represents a single bond. In still another embodiment, the dotted line between C1 and C2 represents a double bond.

In one embodiment of Formula X, the dotted lines between C4 and C5 and between C5 and C6 represent a single bond, and the hydrogen at the C5 bridgehead carbon is in the alpha configuration. In another embodiment, and the dotted lines between C4 and C5 and between C5 and C6 represent a single bond, the hydrogen at the C5 bridgehead carbon is in the beta configuration

In one embodiment of Formula X, R comprises the side chain of a naturally occurring aLamino acid in the L-configuration. In another embodiment, R comprises a residue of L-7 alanine, L-leucine, L-isoleucine, L-proline, or L-valine. In another embodiment, R comprises an amino acid residue with the D-configuration.

In another embodiment, the enantiomers of the compounds of Formula XI are provided. In this embodiment, the stereochemical configuration of each asymmetric carbon is opposite that of the natural steroids and analogues of the natural steroids. For example, the configuration of C9, C10, C13 and C17 carbon atoms would be opposite to the configuration as drawn in the structure above.

In particular embodimcnts of the invention, the neuroprotective steroids will have the formulas nresented in Table 1 below.

TABLE 1

| Com- | |
|---|---|
| pound # | Structure |
| P1-31 | 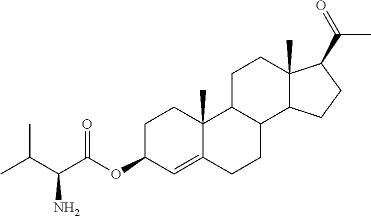 |
| P1-57 | 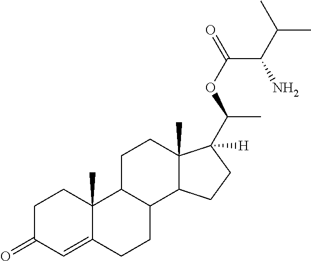 |
| P1-79 | 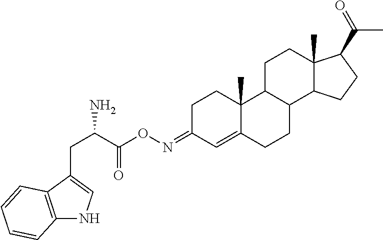 |
| P1-113 | 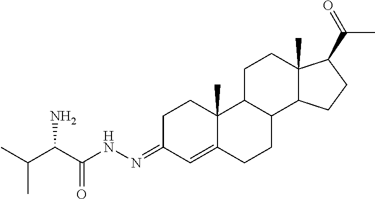 |
| P1-123 | 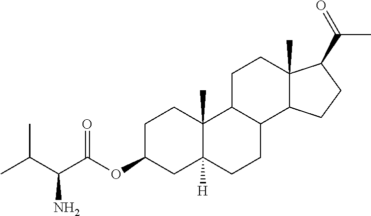 |
| P1-131 | 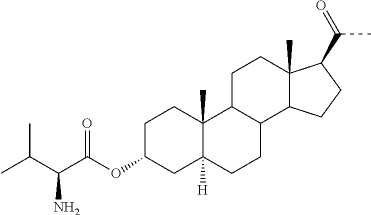 |
| P1-133 | 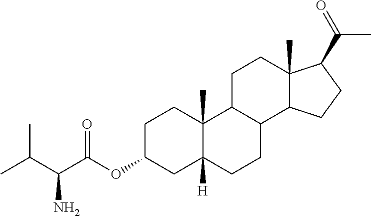 |
| P1-135 | 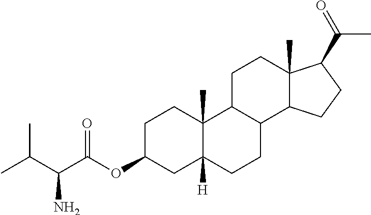 |
| P1-29 | 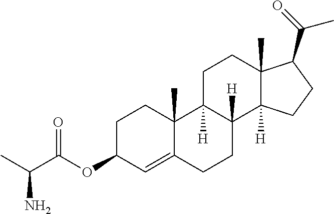 |
| P1-32 | 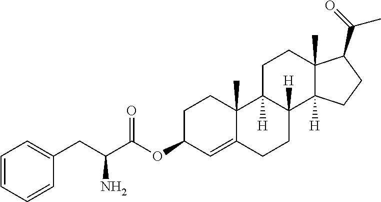 |
| P1-33 | 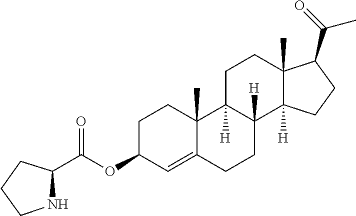 |
| P1-34 | 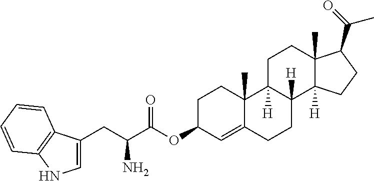 |
| P1-163 | 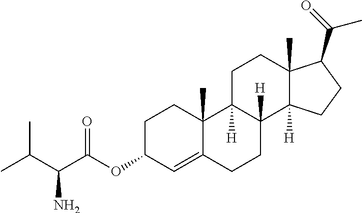 |
| P1-185 | 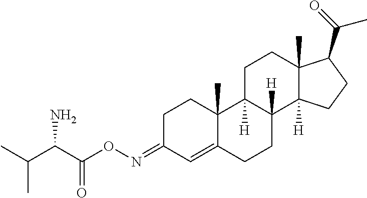 |
| P1-186 | 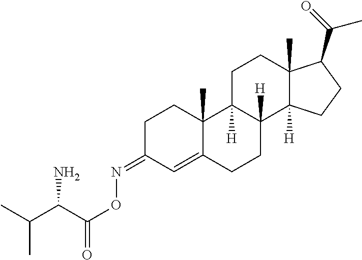 |
| P2-29-E | 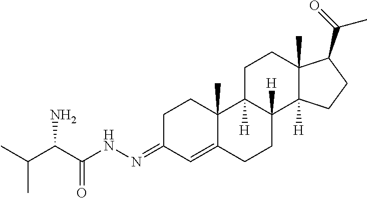 |
| P2-29-Z | 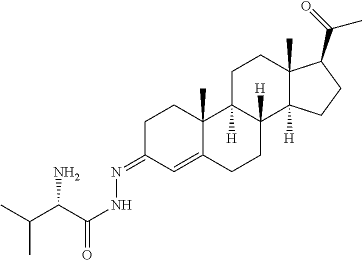 |
| P2-13 | 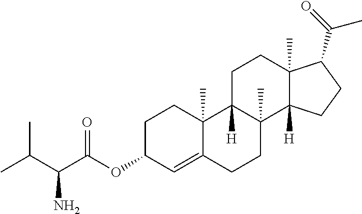 |
| | 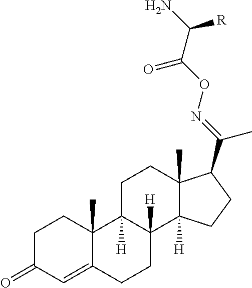 where R is a naturally-occurring amino acid sidechain |
| | 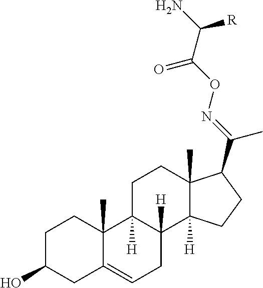 where R is a naturally-occurring amino acid sidechain |
| | 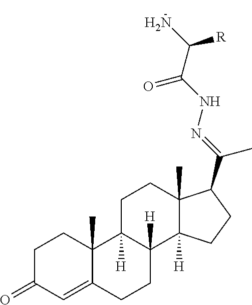 where R is a naturally-occurring amino acid sidechain |
| | 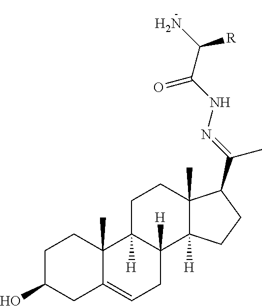 where R is a naturally-occurring amino acid sidechain |
| | 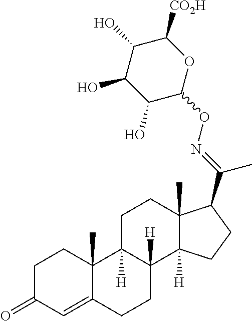 |
| | 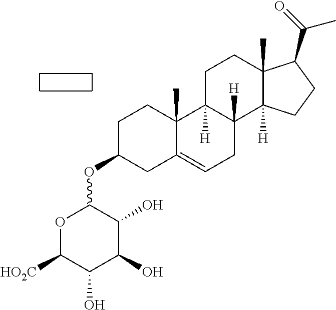 |
| | 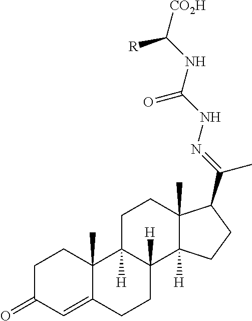 where R is a naturally-occurring amino acid sidechain |
| | 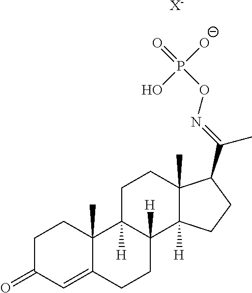 where X⁻ is a counterion |
| | 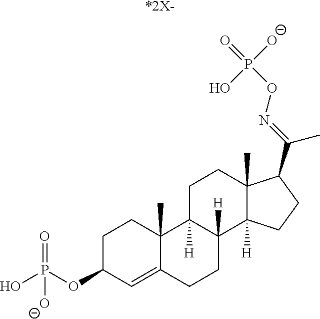 where X⁻ is a counterion, and R is the sidechain of a naturally occurring amino acid |
| | 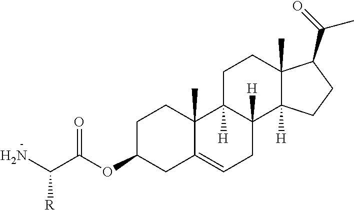 where R is the sidechain of a naturally occurring amino acid |
| | 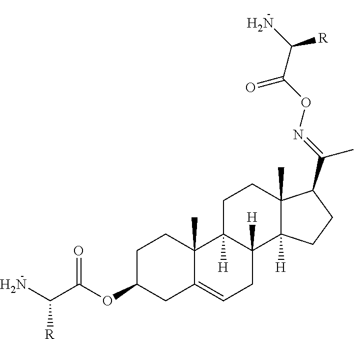 where R is the sidechain of a naturally occurring amino acid |
| | 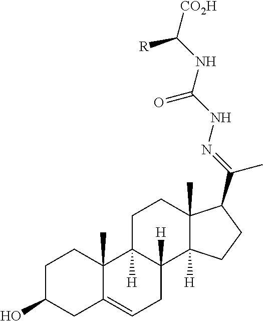 where R is the sidechain of a naturally occurring amino acid |
| | 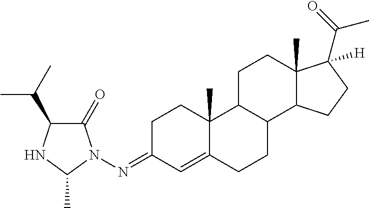 |
| | 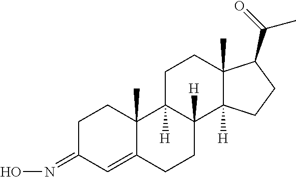 |
| | 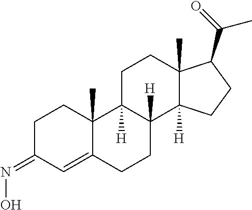 |

In one embodiment of the invention, the pure E- or Z— isomers of the carbonyl-derivatives of the steroid compounds, such as oximes derivatives and the like, are provided. In another embodiment, the invention provides mixtures of E- and Z-isomers of the carbonyl derivatives of the neuroprotective compuonds.

### Stereochemistry

It is understood that based on the number of asymmetric centers, a total number of 2ⁿ possible stereochemical isomers is possible. The present invention includes all possible stereochemical configurations of the compounds.

In some embodiments the stereochemistry of the compounds of the invention will retain the natural stereochemistry of the natural steroid. For example, the stereochemistry at C8, C9, C10, C13, C14 and C17 will retain the stereochemistry of the natural steroid compounds. In contrast, the compounds of the invention include compounds with variable configurations at C-3 and C-5 of the steroid ring system. In some embodiments, the configuration of C-3 is alpha. In other embodiments, the configuration of C-3 is beta. Similarly, in some embodiments, the confirugarion of C-5 is alpha, and in other embodiments the configuration at C-5 is beta. All possible combinations of stereochemical configurations at C-3 and C-5 are embraced by the invention.

In other embodiments, the invention provides enantiomers of the neuroprotective steroids of Formulae I-XI and of the specific compounds in Table 1. In these embodiments, the stereochemical configuration of the asymmetric carbons will be opposite that of the natural steroid compounds.

In a representative embodiment, enantiomers of Formula I of the structure 1-a are provided.

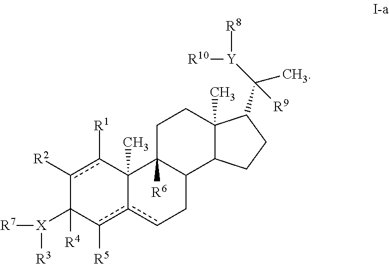

Unless otherwise indicated, the stereochemistry of the compounds of the invention will retain the natural stereochemistry of progesterone at the bridgehead carbon atoms C-8, C-9,C-14 and C-17. In addition, the stereochemistry of the quaternary carbons C-10 and C-13 will also retain the stereochemistry of the progesterone, unless indicated othewise. In contrast, the compounds of the invention include compounds with variable configurations at C₁₋₃ and C-5 of the steroid ring system. In some embodiments, the configuration of C-3 is alpha. In other embodiments, the configuration of C-3 is beta. Similarly, in some embodiments, the confirugarion of C-5 is alpha, and in other embodiments the configuration at C-5 is beta. All possible combinations of stereochemical configurations at C-3 and C-5 are embraced by the invention.

The present invention also encompasses all possible stereochemical configurations of asymmetric substituents, such as amino acids. As described above, the naturally ocurring α-amino acids in L, D, and D,L configurations are encompassed. Furthermore, all possible stereochemical configurations of non-natural synthetic amino acids are encompassed by the invention.

### IV. DEFINITIONS

It should be understood that the various possible stereoisomers of the groups mentioned above and herein are within the meaning of the individual terms and examples, unless otherwise specified. As an illustrative example, “1-methyl-butyl” exists in both the (R) and the (S) form, thus, both (R)-1-methyl-butyl and (S)-1-methyl-butyl is covered by the term “1-methyl-butyl,” unless otherwise specified. Several biological compounds are designed by the (D) and the (L) form, rather than the (R) and the (S) form, respectively. As an another illustrative example, “glycine” exists in both the (D) and the (L) form; therefore, both (D)-glycine and (L)-glycine are covered by the term “glycine” unless otherwise specified.

The term “patient” as used herein is also synonymous with the term “host” and includes any animal. In particular, the term is intended to identify those animals in need of the treatments described herein, whether to treat disease or injury, prevent disease or injury, or maintain health. Although in many embodiments the patient is a human, other animals and in particular mammals are also encompassed in the invention.

As used herein, the term “isolated enantiomer” refers to a composition that includes at least approximately 95% to 100%, or more preferably, over 97% of a single enantiomer of that compound.

As used herein, the term “substantially free of” or “substantially in the absence of” refers to a composition that includes at least 85 or 90% by weight, preferably 95% to 98% by weight, and even more preferably 99% to 100% by weight, of the designated enantiomer of that compound.

The term “independently” is used herein to indicate that the variable that is independently applied varies independently from application to application. Thus, in a compound such as R″XYR″, wherein R¹¹ is “independently carbon or nitrogen,” both R″ can be carbon, both R″ can be nitrogen, or one R″ can be carbon and the other R″ nitrogen.

The term “alkyl,” as used herein unless otherwise specified, is intended to have its customary meaning in the art and includes optionally substituted saturated straight, branched, or cyclic, primary, secondary, or tertiary hydrocarbons. Alkyl, for example, includes methyl, ethyl, propyl, isopropyl, butyl, isobutyl, t-butyl, pentyl, cyclopentyl, isopentyl, neopentyl, hexylisohexyl, cyclohexyl, cyclohexylmethyl, 3-methylpentyl, 2,2-dimethylbutyl and 2,3-dimethylbutyl. The alkyl group can be optionally substituted with one or more moieties. Examples of suitable substituents include alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, thiol, imine, sulfonic acid, sulfate, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphate, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “protected” as used herein and unless otherwise defined refers to a group that is added to an oxygen, nitrogen, sulfur or phosphorus atom to prevent its further reaction or for other purposes. A wide variety of oxygen and nitrogen protecting groups are known to those skilled in the art or organic synthesis. Suitable protecting groups are described, for example, in Greene, et al., “Protective Groups in Organic Synthesis,” John Wiley and Sons, Second Edition, 1991, hereby incorporated by reference.

The term “aryl,” as used herein, is intended to have its customary meaning in the art and includes, for example, phenyl, biphenyl, and naphthyl and the like. The aryl group can be optionally substituted. Non-limiting examples of substituents include hydroxyl, amino, amido, alkylamino, dialkylamino, haloalkyl, arylamino, alkoxy, aryloxy, halo, nitro, cyano, sulfonic acid, thiol, imine, sulfonyl, sulfonyl, fulfinyl, fulfamonyl, ester, sulfate, phosphonic acid, phosphate, phosphonyl, phosphinyl, phosphoryl, phosphonate, phosphine, thioester, thioether, acid halide, anhydride, oxime, hydrozine, carbamate or carboxyl, either unprotected, or protected as necessary, as known to those skilled in the art, for example, as taught in Greene, et al., “Protective Groups in Organic Synthesis,” John Wiley and Sons, Second Edition, 1991.

The term “aralkyl,” as used herein, and unless otherwise specified, refers to an optionally substituted aryl group as defined above linked to the molecule through an alkyl group as defined above. The term alkaryl or alkylaryl as used herein, and unless otherwise specified, refers to an alkyl group as defined above linked to the molecule through an aryl group as defined above. In each of these groups, the alkyl group can be optionally substituted as describe above and the aryl group can be optionally substituted as described above or with any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference. Specifically included within the scope of the term aryl are phenyl; naphthyl; phenylmethyl; phenylethyl; 3,4,5-trihydroxyphenyl; 3,4,5-trimethoxyphenyl; 3,4,5-triethoxyphenyl; 4-chlorophenyl; 4-methylphenyl; 3,5-di-tertiarybutyl-4-hydroxyphenyl; 4-fluorophenyl; 4-chloro-1-naphthyl; 2-methyl-1-naphthylm ethyl; 2-n aphthylmethyl; 4-chlorophenylmethyl; 4-tertiarybutylphenyl; 4-tertiarybutylphenylmethyl and the like.

The term “halo” or “halogen,” as used herein includes chloro, bromo, iodo and fluoro.

The term “heteroatom,” as used herein, refers to oxygen, sulfur, nitrogen or phosphorus.

The term “alkylamino” or “arylamino” refers to an amino group that has one or two alkyl or aryl substituents, respectively.

The term “alkoxy,” as used herein, and unless otherwise specified, refers to a moiety of the structure —O-alkyl, wherein alkyl is as defined above.

The term “acyl” refers to moiety of the formula —C(O)R′, wherein R′ is alkyl, aryl, alkaryl, aralkyl, heteroaromatic, heterocyclic, alkoxyalkyl including methoxymethyl, arylalkyl including benzyl, aryloxyalkyl, such as phenoxymethyl, aryl including optionally substituted phenyl.

As used herein, a “leaving group” means a functional group that is cleaved from the molecule to which it is attached under appropriate conditions.

The term “heteroaryl” or “heteroaromatic,” as used herein are intended to have their customary meaning in the art, and include an aromatic group that includes at least one sulfur, oxygen, nitrogen or phosphorus in the aromatic ring. The term “heterocyclic” refers to a nonaromatic cyclic group wherein there is at least one heteroatom, such as oxygen, sulfur, nitrogen or phosphorus in the ring. Nonlimiting examples of heteroaryl and heterocyclic groups include furyl, furanyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, pyrrole, isopyrrole, pyrazole, or imidazole. The heteroaromatic group can be optionally substituted as described above for aryl. The heterocyclic group can be optionally substituted with one or more moieties. Non-limiting examples of suitable substituents include alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference. The heteroaromatic can be partially or totally hydrogenated as desired. As a nonlimiting example, dihydropyridine can be used in place of pyridine. Functional oxygen and nitrogen groups on the heteroaryl group can be protected as necessary or desired. Suitable protecting groups are well known to those skilled in the art, and include, but are not limited to, 9-fluorenylmethoxycarbonyl (Fmoc), benzyl, trimethylsilyl, dimethylhexylsilyl, t-butyldimethylsilyl, and t-butyldiphenylsilyl, trityl or substituted trityl, alkyl groups, acyl groups such as acetyl, benzoyl; and propionyl, methanesulfonyl, and p-toluenesulfonyl.

Unless otherwise specified, the term “amino acid” includes naturally occurring and synthetic α, β γ or δ amino acids. The naturally occurring amino acids are glycine, alanine, valine, leucine, isoleucine, methionine, phenylalanine, tryptophan, proline, serine, threonine, cysteine, tyrosine, asparagine, glutamine, aspartate, glutamate, lysinc, arginine and histidine. In certain embodiments, the amino acid is in the L-configuration. Alternatively, the amino acid can be a derivative of alanyl, valinyl, leucinyl, isoleuccinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleuccinyl, β-prolinyl, β-phenylalaninyl, β-tryptophanyl, β-methioninyl, β-glycinyl, β-serinyl, β-thrconinyl, β-cysteinyl, β-tyrosinyl, β-asparaginyl, β-glutaminyl, β-aspartoyl, β-glutaroyl, β-lysinyl, β-argininyl or β-histidinyl. When the term amino acid is used, it is considered to be a specific and independent disclosure of each of the esters of α, β γ or δ glycine, alanine, valine, leucinc, isoleucine, methionine, phenylalanine, tryptophan, proline, serine, threonine, cysteine, tyrosine, asparagine, glutamine, aspartate, glutamate, lysine, arginine and histidine in the D and L-configurations.

The term “thio” refers to a sulfur covalently bound to a hydrogen or a carbon based group. Some non-limiting examples include methylmercapto, ethylmercapto, n-propylmercapto, isopropylmercapto or n-butylmercapto, ethylthio, n-propylthio or isopropylthio group. The thio group also can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozinc, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “ester” refers to a carbonyl flanked by an alkoxy group and a carbon based group. Some non-limiting examples include hydroxycarbonyl, methoxycarbonyl, ethoxycarbonyl, n-propyloxycarbonyl, isopropyloxycarbonyl, n-butyloxycarbonyl, isobutyloxycarbonyl, tert-butyloxycarbonyl or 1-(cinnamyloxycarbonyloxy)-ethoxy-carbonyl. Esters of amino acids, as used herein, include groups where a carboxyl group of the amino acid forms an ester bond with a hydroxyl group of the molecule. Also included are groups where a hydroxyl group on the amino acid forms a ester bond with a carboxyl group on the molecule. The ester group also can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

### V. PHARMACEUTICALLY ACCEPTABLE SALT FORMULATIONS

Modifications of the active compound can affect the bioavailability and rate of metabolism of the active species, thus providing control over the delivery of the active species. Further, the modifications can affect the activity of the compound, in some cases increasing the activity over the parent compound. This can easily be assessed by preparing the derivative and testing its activity according to the methods described herein, or other method known to those skilled in the art.

In cases where compounds are sufficiently basic or acidic to form stable nontoxic acid or base salts, administration of the compound as a pharmaceutically acceptable salt may be appropriate. The term “pharmaceutically acceptable salts” or “complexes” refers to salts or complexes that retain the desired biological activity of the compounds of the present invention and exhibit minimal undesired toxicological effects.

Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, ketoglutarate and α-glycerophosphate. Suitable inorganic salts may also be formed, including, hydrochloride, sulfate, nitrate, bicarbonate and carbonate salts. Alternatively, the pharmaceutically acceptable salts may be made with sufficiently basic compounds such as an amine with a suitable acid affording a physiologically acceptable anion. Alkali metal (for example, sodium, potassium or lithium) or alkaline earth metal (for example calcium) salts of carboxylic acids can also be made.

Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalcturonic acid; (b) base addition salts formed with metal cations such as zinc, calcium, bismuth, barium, magnesium, aluminum, copper, cobalt, nickel, cadmium, sodium, potassium, and the like, or with a cation formed from ammonia, N,N-dibenzylethylenediamine, D-glucosamine, tetraethylammonium, or ethylenediamine; or (c) combinations of (a) and (b); e.g., a zinc tannate salt or the like. Also included in this definition are pharmaceutically acceptable quaternary salts known by those skilled in the art, which specifically include the quaternary ammonium salt of the formula —NR⁺A⁻, wherein R is as defined above and A is a counterion, including chloride, bromide, iodide, —O-alkyl, toluenesulfonate, methylsulfonate, sulfonate, phosphate, or carboxylate (such as benzoatc, succinate, acetate, glycolate, maleate, malate, citrate, tartrate, ascorbatc, benzoate, cinnamoate, mandeloate, benzyloate, and diphenylacetate).

Pharmaceutically acceptable prodrugs refer to a compound that is metabolized, for example hydrolyzed or oxidized, in the patient to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound.

Any of the compounds described herein can be administered as a prodrug to increase the activity, bioavailability, stability or otherwise alter the properties of the compound. A number of prodrug ligands are known. In general, alkylation, acylation or other lipophilic modification of the compound will increase the stability of the compound. Examples of substituent groups that can replace one or more hydrogens on the compound are alkyl, aryl, steroids, carbohydrates, including sugars, 1,2-diacylglycerol and alcohols. Many are described in R. Jones and N. Bischofberger, Antiviral Research, 27 (1995) 1-17. Any of these can be used in combination with the disclosed compounds to achieve a desired effect.

### VI. TREATMENT OF CNS INJURIES

The present invention provides methods and compositions for the treatment or prevention of neurodegeneration following an injury to the central nervous system or due to certain neurodegenerative disorders, comprising administering an effective amount of a neuroprotective steroid in combination or alternation with a vitamin D, or a pharmaceutically acceptable salt, ester or prodrug thereof. Multiple physiological events lead to neurodegeneration. These events include, for example, increase in the immune and inflammatory response, demyelinization, and lipid peroxidation. The present invention provides compositions and methods for reducing or eliminating neuronal cell death, edema, ischemia, and enhancing tissue viability following injury to the central nervous system or certain disorders. The analogues, salts, esters or prodrugs of the steroid or secosteroid analogs may be optionally administered with a pharmaceutically acceptable carrier or diluent.

By “treatment or prevention” is intended in some embodiments to mean any enhanced survival, proliferation, and/or neurite outgrowth of the neurons that either prevents or retards neurodegeneration, the progressive loss of neurons. As used herein, “neuroprotection” is the prevention, arrest or reverse progression of neurodegeneration following a central nervous system injury. The neuroprotective effect includes both improved morphological (i.e., enhanced tissue viability) and/or behavioral recovery. CNS injuries that are encompassed within the scope of treatment of the present invention include both traumatic injuries, in particular traumatic brain injury (TBI), and physiological insults such as an ischemic or hemorrhagic stroke. In both instances, a progressive loss of neurons after the initial insult occurs and can be alleviated by use of the inventive compounds, compositions and methods. In accordance with some embodiments, the nervous system injury to be treated or prevented may include neurodegenerative reactions to injury or disease, traumatic brain injury, ischemic CNS injury, hemorrhagic CNS injury, spinal cord injury, ischemic stroke, hemorrhagic stroke and anterior optic nerve ischemic injury. The compositions and methods may achieve one or more effects such as (i) reduced ncurodcgeneration due to apoptosis; (ii) enhanced motor function, (iii) reduced loss of motor function, (iv) reduced inflammation, (v) reduced loss of visual function, and (vi) reduced damage from an inflammatory process.

In some embodiments, a method of treatment or prevention of a nervous system injury is provided that includes administering a neuroprotective steroid in combination or alternation with a vitamin D to a patient suffering from, or at risk of suffering from, such an injury. In certain embodiment, the neuroprotective steroid is a progesterone analog or prodrug. In specific embodiments, the neuroprotective steroid is progesterone or allopregnanolone. In certain embodiments, the vitamin D is 1,25-dihydroxyvitamin D₃ (1,25-diOH-D). The nervous system injury can be a traumatic brain injury, but in other embodiments the injury is an ischemic injury such as a stroke, or any of the other injuries noted above. In certain embodiments, physical damage to neurons is avoided or reduced. The method may enhance physical recovery or reduce loss of function, in particular as related to behavioral or motor function in the patient. Additionally or alternatively, the method may achieve any one or more of the effects noted above.

In specific embodiments of the invention, methods of treating or preventing damage resulting from a nervous system injury, such as from inflammatory processes that are initiated by a TBI, are provided comprising administering a vitamin D in combination or alternation with a neuroprotective neuroprotective steroid or a pharmaceutically acceptable salt, ester or prodrug thereof in a pharmaceutically acceptable carrier to a patient in need thereof. In certain embodiments, the patient is at suffering from a vitamin D deficiency, or from a vitamin D insufficiency. In specific embodiments, the amount of vitamin D administered is sufficient to reduce or reverse a vitamin D deficiency or vitamin D insufficiency.

In certain embodiments, a method of preventing or reducing inflammatory reactions in a patient is provided that includes administering a neuroprotective steroid in combination or alternation with a vitamin D. In certain embodiments, the patient is at risk of or suffering from vitamin D deficiency. In certain other embodiments, the patients not at risk of vitamin D deficiency. In certain embodiment, the neuroprotective steroid is a progesterone analog. In specific embodiments, the neuroprotective steroid is progesterone. In certain embodiments, the vitamin D is 1,25-diOH-D.

In certain embodiments, methods of neuroprotection are provided comprising administering a vitamin D in combination or alternation with a neuroprotective steroid, its physiologically acceptable salt or prodrug, optionally in a pharmaceutically acceptable carrier, to a patient at risk of suffering from a stroke. In other embodiments, methods of treating or preventing neuronal damage are provided comprising administering a vitamin D in combination or alternation with a neuroprotective steroid or its physiologically acceptable salt or prodrug, optionally in a pharmaceutically acceptable carrier, to a paticnt who has suffered from an ischemic stroke. The method can reduce prevent neurodegeneration such as that caused by excitotoxic or inflammatory reactions, or can enhance neuronal proliferation, growth or differentiation in the period after the injury. In yet further embodiments, methods of treating or preventing cognitive or behavioral deficits after a stroke is provided comprising administering a compound of the invention or its physiologically acceptable salt or prodrug, optionally in a pharmaceutically acceptable carrier, to a patient who has suffered a stroke. In certain embodiments, the stroke is an ischemic stroke, but it can alternatively be a hemorrhagic stroke.

In other embodiments, the present invention provides a method to achieve a neuroprotective effect following a traumatic CNS injury in a mammal, in particular in a human, comprising administering a therapeutically effective amount of a vitamin D in combination or alternation with a neuroprotective steroid to a patient following a traumatic CNS injury. A traumatic injury to the CNS is characterized by a physical impact to the central nervous system. The physical forces resulting in a traumatic brain injury cause their effects by inducing three types of injury: skull fracture, parenchymal injury, and vascular injury. A blow to the surface of the brain typically leads to rapid tissue displacement, disruption of vascular channels, and subsequent hemorrhage, tissue injury and edema. Morphological evidence of injury in the neuronal cell body includes pyknosis of nucleus, eosinophilia of the cytoplasm, and disintegration of the cell. Furthermore, axonal swelling can develop in the vicinity of damage neurons and also at great distances away from the site of impact.

In certain embodiments, the vitamin D and neuroprotective steroid is administered within six hours after onset of a stroke or after an injury, such as a TBI. In some embodiments, the vitamin D and neuroprotective steroid are administered within three hours of a TBI, stroke or other injury to the brain, such as within two or one hour. In some other embodiments, the compounds are administered within one day (i.c. 24 hours) of the injury, or within any other timeframe described herein above and below. In certain embodiments, the compounds are provided to individuals at risk of a stroke, such as those who are suffering from atherosclerosis or have a family history of heart disease. In other embodiments, the compounds are provided to individuals at risk of any other injury or disease discussed herein, such as those whose work, status or lifestyle places them at risk for nervous system injury, such as CNS injury or TBI, such as athletes and soldiers. These compounds can be provided to individuals as a preventative therapy to descrease neural trauma.

In another embodiment, a method for decreasing ischemia following a brain injury is provided comprising administering an effective amount of a vitamin D in combination or alternation with a neuroprotective steroid to a patient suffering from a brain injury. The methods of the invention provide a means to reduce or eliminate the inflammatory immune reactions that follow a CNS injury. By reducing the inflammatory response, the combinations of the present invention can substantially reduce brain swelling and reduce the amount of neurotoxic substances (e.g., free radicals and excitotoxins) that are released from the site of injury.

The present invention provides for a method of treating a brain injury by administering to a subject a vitamin D in combination or alternation with a neuroprotective steroid, a pharmaceutically acceptable salt or a prodrug or ester thereof. The concentration of the neuroprotective steroid and vitamin D, or salt, ester or prodrug thereof, in accordance with the present invention may be effective in the treatment or prevention of neuronal damage that follows either a traumatic, ischemic or hemorrhagic injury to the CNS and hence, elicit a neuroprotective effect. The therapeutically effective amount will depend on many factors including, for example, the specific activity of the neuroprotective steroid administered, the type of injury, the severity and pattern of the injury, the resulting neuronal damage, the responsiveness of the patient, the weight of the patient along with other intraperson variability, the method of administration, and the formulation used.

It is recognized that a traumatic injury to the CNS results in multiple physiological events that impact the extent and rate of neurodegeneration, and thus the final clinical outcome of the injury. The treatment of a traumatic injury to the CNS encompasses any reduction and/or prevention in one or more of the various physiological events that follow the initial impact. For example, cerebral edema frequently develops following a traumatic injury to the CNS and is a leading cause of death and disability. Cortical contusions, for example, produce massive increases in brain tissue water content which, in turn, can cause increased intracranial pressure leading to reduced cerebral blood flow and additional neuronal loss. Hence, the methods of the invention find use in reducing and/or eliminating cerebral edema and/or reducing the duration of the edemic event following a traumatic injury to the CNS. Assays to determine a reduction in edema are known in the art and include, but are not limited to, a decrease in tissue water content following the administration of the compounds (Betz et al. (1990) Stroke 21:1199-204). Furthermore, an overall improvement in behavioral recovery can also be used as a measure for a decrease in edema. A decrease in edema in the effected tissue by at least about 15% to 30%, about 30% to 45%, about 45% to 60%, about 60% to 80%, or about 80% to 95% or greater will be therapeutically beneficial, as will any reduction in the duration of the edemic event.

Further physiological effects of brain injury include an inflammatory response. In particular, some studies indicate that the acute inflammatory response contributes significantly to injury after ischemia (see Perera, et al. (2005) Inflammation following stroke. J. Clin. Neurosc. 13:1-8; Barone and Feuerstein (1999) Inflammatory mediators and stroke: new opportunities for novel therapeutics). The stroke process triggers an inflammatory reaction that may last up to several months. Suppression of inflammation can reduce infarct volume and improve clinical outcomes even with the initiation of therapy after 3 hours of onset of stroke. In addition, an immune response can be triggered both by strokes. Infiltrating leukocytes are thought to contribute to secondary ischemic damage by producing toxic substances that kill brain cells and disrupt the blood-brain barrier (see del Zoppo, et al. (2000) Advances in the vascular pathophysiology of ischemic stroke. Thromb Res. 98:73-81) Infiltration occurs when leukocytes bind endothelial intercellular adhesion molecule-1 (ICAM-1) and ICAM-1 is upregulated after ischemia.

TBI also elicits inflammatory, and in particular a immune responses. See, for example, Soares et al. (1995) J. Ncurosci. 15:8223-33; Holmin et al. (1995) Acta Neurochir. 132:110-9; Arvin et al. (1996) Neurosci. Biobehay. Rev. 20:445-52. Following a cortical impact, severe inflammatory reactions and gliosis at the impact site and at brain areas distal to the primary site of injury occurs. The inflammatory response is characterized by the expression of adhesion molecules on the vascular surfaces, resulting in the adherence of immune cells and subsequent extravasation into the brain parenchyma. By releasing cytokines, the invading macrophages and neutrophils stimulate reactive astrocytbsis. Release of different chemokines by other cell types induces these immune cells to become phagocytic, with the simultaneous release of free radicals and pro-inflammatory compounds, e.g., cytokines, prostaglandins, and excitotoxins (Arvin et al. (1996) Ncurosci. Biobchay. Ref. 20:445-52; Raivich et al. (1996) Kelo J. Med. 45:239-47; Mattson et al. (1997) Brain Res. Rev. 23:47-61; all of which are herein incorporated by reference).

Assays for assessing the efficacy of the compounds described herein include assays to determine a decrease in an ischemic event include, for example, a decrease in infarct area, improved body weight, and improved neurological outcome. Assays to measure a reduction in lipid peroxidation in both brain homogenate and in mitochondria are known in the art and include, for example, the thiobarhituric acid method (Roof et al. (1997) Mol. Chem. Neuropathol. 31: 1-11; Subramanian et al. (1993) Neurosci. Lett. 155:151-4; Goodman et al. (1996) J. Neurochem. 66:1836-44; Vedder et al. (1999) J. Neurochem. 72:2531-8; all of which are herein incorporated by reference) and various in vitro free radical generating systems Furthermore, alterations in the levels of critical free radical scavenger enzymes, such as mitochondrial glutathione can be assayed. See, for example, Subramanian et al. (1993) Neurosci. Lett. 155:151-4; and Vedder et al. (1999) J. Neurochem. 72:2531-8; both of which are herein incorporated by reference.

Methods to quantify the extent of central nervous system damage (i.e., neurodegeneration) and to determine if neuronal damage was treated or prevented following the administration of the compositions described herein are well known in the art. Such neuroprotective effects can be assayed at various levels, including, for example, by promoting behavioral and morphological (i.e., enhancing tissue viability) recovery after traumatic brain injury. A variety of anatomical, immunocytochemical and immunological assays to determine the effect of the neuroprotective steroid on necrosis, apoptosis, and neuronal glial repair are known in the art. As such, the neuroprotection resulting from the methods of the present invention will result in at least about a 10% to 20%, 20% to 30%, 30% to 40%, 40% to 60%, 60% to 80% or greater increase in neuronal survival and/or behavioral recovery as compared to the control groups.

Histological and molecular marker assays for an increase in neuronal survival are known. For example, Growth Associated Protein 43 (GAP-43) can be used as a marker for new axonal growth following a CNS insult. See, for example, Stroemer et al. (1995) Stroke 26:2135-2144, Vaudano et al. (1995) J. of Neurosci 15:3594-3611. Other histological markers can include a decrease in astrogliosis and microgliosis. Alternatively, a delay in cellular death can be assayed using TUNEL labeling in injured tissue. Further anatomical measures that can be used to determine an increase in neuroprotection include counting specific neuronal cell types to determine if the neuroprotective steroid is preferentially preserving a particular cell type (e.g., cholinergic cells) or neurons in general.

In addition, behavioral assays can be used to determine the rate and extent of behavior recovery in response to the treatment. Improved patient motor skills, spatial learning performance, cognitive function, sensory perception, speech and/or a decrease in the propensity to seizure may also be used to measure the neuroprotective effect. Such functional/behavioral tests used to assess sensorimortor and reflex function are described in, for example, Bcderson et al. (1986) Stroke 17:472-476, DeRyck et al. (1992) Brain Res. 573:44-60, Markgraf et al. (1992) Brain Res. 575:238-246, Alexis et al. (1995) Stroke 26:2336-2346; all of which are herein incorporated by reference. Enhancement of neuronal survival may also be measured using the Scandinavian Stroke Scale (SSS) or the Barthl Index. Behavioral recovery can be further assessed using the recommendations of the Subcommittee of the NIH/NINDS Head Injury Centers in Humans (Hannay et al. (1996) J. Head Trauma Rehabil. 11:41-50), herein incorporated by reference. Behavioral recovery can be further assessed using the methods described in, for example, Beaumont et al. (1999) Neurol Res. 21:742-754; Becker et al. (1980) Brain Res. 200:07-320; Buresov et al. (1983) Techniques and Basic Experiments for the Study of Brain and Behavior; Kline et al. (1994) Pharmacol. Biochem. Behay. 48:773-779; Lindner et al. (1998) J. Neurotrauma 15:199-216; Morris (1984) J. Neurosci. Methods 11:47-60; Schallert et al. (1983) Pharmacol. Biochem. Behay. 18:753-759.

Assays that can be used to determine if the combinations described herein are imparting an anti-inflammatory and a nonspecific suppressive effect on the immune system following a injury include, for example, a reduction in cytokine induced microglial proliferation in vitro (Hoffman et al. (1994) J. Neurotrauma 11:417-31; Garcia-Estrada et al. (1993) Brain Res. 628:271-8; both of which are herein incorporated by reference); a reduction in the generation of cytotoxic free radicals by activated macrophages (Chao et al. (1994) Am. J. Reprod. Immunol. 32:43-52; Robert et al. (1997) Nitric Oxide 1:453-62; Kelly et al. (1997) Biochem. Biophys. Res. Commun. 239:557-61; Ganter et al. (1992) J. Neurosci. Res. 33:218-30; all of which are herein incorporated by reference); a reduction in the expression of inducible nitric oxide synthetase and the amount of nitric oxide release by macrophages (Robert et al. (1997) Nitric Oxide 1:453-62; Miller et al. (1996) J. Leukoc. Biol. 59:442-50; both of which are herein incorporated by reference); the release of a “progesterone-induced blocking factor” that inhibits natural killer cell activity (Check et al. (1997) Am. J. Reprod. Immunol. 37:17-20; Szekeres-Bartho et al. (1997) Cell Immunol. 177:194-9; Szekeres-Bartho et al. (1996) Am. J. Reprod. Immunol. 35:348-51; all of which are herein incorporated by reference); a decrease in the number of GFAP-positive astrocytes after brain injury which is suggestive of less secondary damage (Garcia-Estrada et al. (1993) Brain Res. 628:271-8; Garcic-Estrada et al. (1999) Int. J. Dev. Neurosci. 17:145-51; Cheek et al. (1997) Am. J. Reprod. Immunol. 37:17-20; Szekeres-Bartho et al. (1997) Cell Immunol. 177:194-9; Szekeres-Bartho et al. (1996) Am. J. Reprod. Immunol. 35:348-51; all of which are herein incorporated by reference); a reduction in the number of inflammatory immune cells (OX42-positive cells); a reduction in the loss of ChAT-positive and COX-positive neurons; a reduction in the number of TUNEL-positive and MnSOD-positive neurons; and an increase in the intensity of succinate dehydrogenase and cytochrome oxidase activity.

Furthermore, a reduction in the inflammatory immune reactions following a traumatic brain injury can be assayed by measuring the cytokines level following the injury in the sham controls versus the treated subjects. Cytokines are mediators of inflammation and are released in high concentrations after brain injury. The level of pro-inflammatory cytokines (e.g., interleukin 1-beta, tumor necrosis factor, and interleukin 6) and the level of anti-inflammatory cytokines (e.g., interleukin 10 and transforming growth factor-beta) can be measured. For instance, “real-time” polymerase chain reactions (PCR) can be used to measure the strength of the mRNA signal and ELISA can be used to determine protein levels. In addition, histological analysis for different inflammatory cell types (e.g., reactive astrocytes, macrophages and microglia) can be used to measure a reduction in the inflammatory response.

The compositions and methods of the invention can also have potential for use in other disorders including multiple sclerosis, catamenial epilepsy, diabetic neuropathy, inflammatory disorders (e.g., rheumatoid arthritis, inflammatory bowel disease), hemorrhagic shock, Niemann-Pick disorder, cerebral palsy, and congenital heart disorders.

In specific embodiments, a method of treatment or prevention of neural degeneration related to Amyotrophic Lateral Sclerosis (ALS), is provided comprising administering a vitamin D in combination or alternation with a neuroprotective steroid to a patient suffering from or at risk of suffering from ALS. ALS, more commonly known as Lou Gehrig's Disease, strikes both males and females, typically between the ages of 40 and 70. This is a motor neuron disorder in which both the upper and lower motor neurons are affected. Patients' muscles atrophy as the motor neurons cease sending signals to initiate movement. This affects not only muscles required for locomotion but also the muscles used in swallowing. Up until the age of 60, males are disproportionally affected at a ratio of 1.5 to 1. After the age of 60, the numbers are equal across genders. The incidence of ALS is approximately ½ that of multiple sclerosis. Life expectancy post-diagnosis is 2-5 years. There are 120,000 cases of ALS diagnosed worldwide and 350,000 patients coping with the disease at any given time. A treatment for ALS will clearly qualify for orphan drug status. The cause of ALS has not been identified. The pathogencsis is poorly understood but cxcitotoxicity, inflammation, oxidative stress and protein aggregation have been shown. In some cases, super oxide dismutase 1 (SOD1) has been determined to be aberrant. Glutamate toxicity is now generally accepted as part of AS pathology. Progesterone has proven to protect neurons from the effects of this toxicity. The only compound approved for the treatment of ALS is Rilutek™ which may reduce glutamate levels. It is not curative but has reduced the rate of progression in some patients.

In another specific embodiments, a method of treatment or prevention of neural degeneration related to Parkinson's Disease (PD), is provided comprising administering a vitamin D in combination or alternation with a neuroprotective steroid to a patient suffering from or at risk of suffering from PD. PD is a neurodegenerative disease of unknown etiology that results in the progressive loss of nerve cell function in the brain. Life expectancy is 15-25 years post-diagnosis; however, there is no cure. It is estimated that one million people in the U.S. are living with Parkinson's; a number that is greater than the combined total of multiple sclerosis, muscular dystrophy and amyotrophic lateral sclerosis patients. The incidence of PD increases with age. Nearly 40,000 people are diagnosed each year with PD, of which ˜15% will be less than 50 years in age. The cost of PD annually exceeds $25 billion when both direct and indirect costs are combined. In PD, cells in the substania nigra of the brain cease to function properly and die. These cells produce dopamine, a neurotransmitter. Dopamine regulates those parts of the brain which control the initiation of movement and coordination. Without dopamine, a patient will begin to experience tremors, bradykinesia, postural instability, rigidity of limbs and trunk, and/or impaired balance and coordination. Not all patients experience all symptoms nor do they progress at the same rate. PD is ultimately debilitating for many sufferers who require assistance in everyday living.

In another specific embodiments, a method of treatment or prevention of neural degeneration related to spinal cord trauma is provided comprising administering a vitamin D in combination or alternation with a neuroprotective steroid to a patient in need thereof. In another specific embodiments, a method of treatment or prevention of neural degeneration related to hypoxia is provided comprising administering a vitamin D in combination or alternation with a neuroprotective steroid to a patient in need thereof.

### VII. COMBINATION AND ALTERNATION THERAPY

In further embodiments of the present invention, the compositions of the invention may be administered in combination or alternation with at least one additional neuroprotective agent to enhance neuroprotection following a traumatic CNS injury. In one embodiment, the neuroprotective steroids of the invention may be administered in combination or alternation with other steroid anaologues or with progesterone.

Other neuroprotective agents of interest include, for example, compounds that reduce glutamate excitotoxicity and enhance neuronal regeneration. Such agents may be selected from, but not limited to, the group comprising growth factors. By “growth factor” is meant an extracellular polypeptide-signaling molecule that stimulates a cell to grow or proliferate. Preferred growth factors are those to which a broad range of cell types respond. Examples of neurotrophic growth factors include, but are no limited to, fibroblast growth factor family members such as basic fibroblast growth factor (bFGF) (Abraham et al. (1986) Science 233:545-48), acidic fibroblast growth factor (aFGF) (Jaye et al. (1986) Science 233:541-45), the hst/Kfgf gene product, FGF-3 (Dickson et al. (1987) Nature 326-833), FGF-4 (Zhan et al. (1988) Mol. Cell. Biol. 8:3487-3495), FGF-6 (deLapeyriere et al. (1990) Oncogene 5:823-831), keratinocyte growth factor (KGF) (Finch et al. (1989) Science 245:752-755), and androgen-induced growth factor (A1GF) (Tanaka et al. (1992) Proc. Natl. Acad. Sci. USA 89:8928-8923).

Additional neuroprotective agents include, ciliary neurotrophic factor (CNTF), nerve growth factor (NGF) (Seiler, M. (1984) Brain Research 300:33-39; Hagg T. et al. (1988) Exp Neurol 101:303-312; Kromer L. F. (1987) Science 235:214-216; and Hagg T. et al. (1990) J. Neurosci 10(9):3087-3092), brain derived neurotrophic factor (BDNF) (Kiprianova, I. et al. (1999) J. Neurosci. Res. 56:21-27), Neurotrophin 3 (NT3), Neurotrophin 4 (NT4), transforming growth factor-.beta.1 (TGF-.beta.1) (Henrick-Noack, P. et al. (1996) Stroke 27:1609-14), bone morphogenic protein (BMP-2) (Hattori, A. et al. (1999) J. Neurochem. 72:2264-71), glial-cell line derived neurotrophic factor (GDNF) (Miyazaki, H. et al. (1999) Neuroscience 89:643-7), activity-dependant neurotrophic factor (ADNF) (Zamostiano, R. et al. (1999) Neurosci Letter 264:9-12), cytokine leukemia inhibiting factor (LIF) (Blcsch, A. et al. (1999) J. Neurosci. 19:3356-66), oncostatin M, interleukin, and the insulin-like growth factors 1 and 2.

Other forms of neuroprotective therapeutic agents include, for example, Clomethiazolc (Zcndra) (Marshal, J. W. et al. (1999) Exp. Neurol. 156:121-9); kynurenic acid (KYNA) (Salvati, P. et al. (1999) Prog Neruopsychopharmacol Biol Psychiatry 23:741-52), Semax (Miasoedova, N. F. et al. (1999) Zh Nevrol Psikhiatr Imss Korsakova 99:15-19), FK506 (tacrolimus) (Gold, B. G. et al. (1999) J. Pharmacol. Exp. Ther. 289:1202-10), L-threo-1-phenyl-2-decanoylamino-3-morpholino-1-propanol (Inokuchi, J. et al. (1998) Act Biochim Pol 45:479-92), andrenocorticotropin-(4-9) analoge (ORG 2766) and dizolcipine (MK-801) (Herz, R. C. et al. (1998) Eur J. Pharmacol 346:159-65), cerebral interleukin-6) (Loddick, S. A. et al. (1998) J. Cereb Blood Flow Metab 18:176-9), selegiline (Semkova, I. et al. (1996) Eur J. Pharmacol 315:19-30), MK-801 (Barth, A. et al. (1996) Neuro Report 7:1461-4; glutamate antagonist such as, NPS1506, GV1505260, MK801 (Baumgartner, W. A. et al. (1999) Ann Thorac Surg 67:1871-3), GV150526 (Dyker, A. G. et al. (1999) Stroke 30:986-92); AMPA antagonist such as NBQX (Baumgartner, W. A. (1999) et al. Ann Thorac Surg 67:1871-3, PD152247 (PNQX) (Schielke, G. P. et al. (1999) Stroke 30:1472-7), SPD 502 (Nielsen, E. O. et al. (1999) J. Pharmacol Exp Ther 289:1492-501), LY303070 and LY300164 (May, P. C. et al. (1999) Neuroscience Lett 262:219-221).

When the compositions of the present invention are administered in combination or alternation with other pharmaceutically active agents, (i.e., other neuroprotective agents) a lower level of either or both vitamin D or neuroprotective steroid may be used. In particular embodiments, reduced levels of steroids may be used, however a vitamin D will still be provided in equivalent dosages.

The compositions may be administered once or several times a day. The duration of the treatment may be once per day for a period of up to from two to three weeks and may continue for a period of months or even years. The daily dose can be administered either by a single dose in the form of an individual dosage unit or several smaller dosage units or by multiple administration of subdivided dosages at certain intervals.

For example, a dosage unit can be administered from 0 hours to 1 hr, 1 hr to 24 hr or 24 hours to at least 100 hours post injury. Alternatively, the dosage unit can be administered from about 0.5, 1, 2, 3, 4, 5, 6, 7, 8, 9, 10, 11, 12, 13, 14, 15, 16, 17, 18, 19, 20, 21, 22, 23, 24, 30, 40, 48, 72, 96, 120 hours or longer post injury. Subsequent dosage units can be administered any time following the initial administration such that a therapeutic effect is achieved. For instance, additional dosage units can be administered to protect the subject from the secondary wave of edema that may occur over the first several days post-injury.

In combination therapy, effective dosages of two or more agents are administered together, such as in the same composition or in different compositions administered by the same or different routes at about the same time, whereas during alternation therapy an effective dosage of each agent is administered serially, such as at different times on the same day, on different days, and/or according to different dosing schedules. The dosages will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules may be adjusted over time according to the individual need and the professional judgment of the person administering or supervising the administration of the compositions.

The efficacy of a drug can be prolonged, augmented, or restored by administering the compound in combination or alternation with a second, and perhaps third, agent. Alternatively, the pharmacokinetics, biodistribution or other parameter of the drug can be altered by such combination or alternation therapy. In general, combination therapy is typically preferred over alternation therapy because it induces multiple simultaneous stresses on the condition.

In another embodiment, the active compound is administered in combination or alternation with one or more other non-steroidal anti-inflammatory drug(s) (NSAIDS). Examples of NSAIDS that can be used in alternation or combination therapy are carboxylic acids, propionic acids, fenamates, acetic acids, pyrazolones, oxicans, alkanones, gold compounds and others that inhibit prostaglandin synthesis, preferably by selectively inhibiting cylcooxygenase-2 (COX-2). Some nonlimiting examples of COX-2 inhibitors are Celebrex (celecoxib) and Vioxx (rofacoxib). Some non-limiting examples of NSAIDS are aspirin (acetylsalicylic acid), Dolobid (diflunisal), Disalcid (salsalate, salicylsalicylate), Trisilate (choline magnesium trisalicylate), sodium salicylate, Cuprimine (penicillamine), Tolectin (tolmetin), ibuprofen (Motrin, Advil, Nuprin Rufen), Naprosyn (naproxen, Anaprox, naproxen sodium), Nalfon (fenoprofen), Orudis (ketoprofen), Ansaid (flurbiprofen), Daypro (oxaprozin), meclofenamate (meclofanamic acid, Meclomen), mefenamic acid, Indocin (indomethacin), Clinoril (sulindac), tolmetin, Voltaren (diclofenac), Lodine (etodolac), kctorolac, Butazolidin (phenylbutazone), Tandearil (oxyphenbutazone), piroxicam (Feldene), Relafen (nabumetone), Myochrysine (gold sodium thiomalate), Ridaura (auranofin), Solganal (aurothioglucose), acetaminophen, colchicine, Zyloprim (allopurinol), Benemid (probenecid), Anturane (sufinpyrizone), Plaquenil (hydroxychloroquine), Aceclofenac, Acemetacin, Acetanilide, Actarit, Alclofenac, Alminoprofen, Aloxiprin, Aluminium Aspirin, Amfcnac Sodium, Amidopyrine, Aminopropylone, Ammonium Salicylate, Ampiroxicam, Amyl Salicylate, Anirolac, Aspirin, Auranofin, Aurothioglucose, Aurotioprol, Azapropazone, Bendazac (Bendazac Lysine), Benorylate, Benoxaprofen, Benzpiperylone, Benzydamine hydrochloride, Bomyl Salicylate, Bromfenac Sodium, Bufexamac, Bumadizone Calcium, Butibufen Sodium, Capsaicin, Carbaspirin Calcium, Carprofen, Chlorthenoxazin, Choline Magnesium Trisalicylate, Choline Salicylate, Cinmetacin, Clofexamide, Clofezone, Clometacin, Clonixin, Cloracetadol, Cymene, Diacerein, Diclofenac (Diclofenac Diethylammonium Salt, Diclofenac Potassium, Diclofenac Sodium), Diethylamine Salicylate, Diethylsalicylamide, Difenpiramide, Diflunisal, Dipyrone, Droxicam, Epirizole, Etenzamide, Etersalate, Ethyl Salicylate, Etodolac, Etofenamate, Felbinac, Fenbufen, Fenclofenac, Fenoprofen Calcium, Fentiazac, Fepradinol, Feprazone, Floctafenine, Flufenamic, Flunoxaprofen, Flurbiprofen (Flurbiprofen Sodium), Fosfosal, Furprofen, Glafenine, Glucametacin, Glycol Salicylate, Gold Keratinate, Harpagophytum Procumbens, Ibufenac, Ibuprofen, Ibuproxam, Imidazole Salicylate, Indomethacin (Indomethacin Sodium), Indoprofen, Isamifazone, Isonixin, Isoxicam, Kebuzone, Ketoprofen, Ketorolac Trometamol, Lithium Salicylate, Lonazolac Calcium, Lomoxicam, Loxoprofen Sodium, Lysine Aspirin, Magnesium Salicylate, Meclofenamae Sodium, Mefenamic Acid, Meloxicam, Methyl Butetisalicylate, Methyl Gentisate, Methyl Salicylate, Metiazinic Acid, Metifenazone, Mofebutazone, Mofezolac, Morazone Hydrochloride, Momiflumate, Morpholine Salicylate, Nabumetone, Naproxen (Naproxen Sodium), Nifenazone, Niflumic Acid, Nimesulide, Oxametacin, Oxaprozin, Oxindanac, Oxyphenbutazone, Parsalmide, Phenybutazone, Phenyramidol Hydrochloride, Picenadol Hydrochloride, Picolamine Salicylate, Piketoprofen,

Pirazolac, Piroxicam, Pirprofen, Pranoprofen, Pranosal, Proglumetacin Maleate, Proquazone, Protizinic Acid, Ramifenazone, Salacetamide, Salamidacetic Acid, Salicylamide, Salix, Salol, Salsalate, Sodium Aurothiomalate, Sodium Gentisate, Sodium Salicylate, Sodium Thiosalicylate, Sulindac, Superoxide Dismutase (Orgotein, Pegorgotein, Sudismase), Suprofen, Suxibuzone, Tenidap Sodium, Tenoxicam, Tetrydamine, Thurfyl Salicylate, Tiaprofenic, Tiaramide Hydrochloride, Tinoridine Hydrochloride, Tolfenamic Acid, Tometin Sodium, Triethanolamine Salicylate, Ufenamate, Zaltoprofen, Zidometacin and Zomepirac Sodium.

### VIII. PHARMACEUTICAL COMPOSITIONS

In one embodiment, a pharmaceutical composition is provided that includes a vitamin D in combination with a neuroprotective steroid. In particular embodiments, the vitamin D is provided in an amount effective to reverse vitamin D deficiency, or to reverse vitamin D insufficienct. In specific embodiments, the vitamin D is selected from ergocalciferol, Seocalcitol and cholecalciferol. In specific embodiments, the effective amount is at least 1000 international units (IU) per day, or at least 1500 IU/day, or at least or at least 2000 IU/day, or at least 2500 IU/day, or at least 3000 IU/day, or at least 3500 IU/day, or at least 4000 IU/day, at least 5000 IU/day, at least 10,000 IU/day, at least 25,000 IU/day or at least 50,000 IU/day, or greater. In specific embodiments, the effective amount of neuroprotective steroid is from about 0.1 mg to about 100 mg per kilogram of body weight per day, or from about 0.5 mg to about 50 mg per kilogram of body weight per day, or from about 0.25 gram to about 3.0 grams of the active compound for a subject of about 70 kg of body weight are administered in a 24-hour period. In certain embodiments, the composition is provided for oral or nasal administration, however in other embodiments, the composition is provided for intravenous or intramuscular administration.

The described compounds can be formulated as pharmaceutical compositions and administered for the treatment or prevention of CNS injury, and particularly traumatic brain injury. The compositions can be administered in any of a variety of forms adaptcd to the chosen route of administration, including systemically, such as orally or nasally, or parenterally, by intravenous, intramuscular, topical, transdermal or subcutaneous routes.

The compounds can be included in the pharmaceutically acceptable carrier or diluent in an amount sufficient to deliver to a patient a therapeutically effective amount of compound to treat traumatic CNS injury in vivo without causing serious toxic effects in the patient treated.

The steroid and vitamin D compositions used in the methods of the invention may further comprise an inorganic or organic, solid or liquid, pharmaceutically acceptable carrier. The carrier may also contain preservatives, wetting agents, emulsifiers, solubilizing agents, stabilizing agents, buffers, solvents and salts. Compositions may be sterilized and exist as solids, particulants or powders, solutions, suspensions or emulsions.

The steroid and vitamin D compositions can be formulated according to known methods to prepare pharmaceutically useful compositions, such as by admixture with a pharmaceutically acceptable carrier vehicle. Suitable vehicles and their formulation are described, for example, in Remington's Pharmaceutical Sciences (16th ed., Osol, A. (ed.), Mack, Easton Pa. (1980)). In order to form a pharmaceutically acceptable composition suitable for effective administration, such compositions will contain an effective amount of the compound, either alone, or with a suitable amount of carrier vehicle.

The pharmaceutically acceptable carrier of the present invention will vary depending on the method of drug administration. The pharmaceutical carrier employed may be, for example, either a solid, liquid, or time release. Representative solid carriers are lactose, terra alba, sucorse, talc, geletin, agar, pectin, acacia, magnesium stearate, stearic acid, microcrystallin cellulose, polymer hydrogels, and the like. Typical liquid carriers include syrup, peanut oil, olive oil, cyclodextrin, and the like emulsions. Those skilled in the art are familiar with appropriate carriers for each of the commonly utilized methods of administration. Furthermore, it is recognized that the total amount of neuroprotective steroid administered as a therapeutic effective dose will depend on both the pharmaceutical composition being administered (i.e., the carrier being used) and the mode of administration.

In one embodiment, a steroid and/or vitamin D, or their pharmaceutically acceptable salt, ester or prodrugs, is administered via parenteral administration in a dose of about 0.1 ng to about 100 g per kg of body weight, about 10 ng to about 50 g per kg of body weight, from about 100 ng to about 1 g per kg of body weight, from about 1 ug to about 100 mg per kg of body weight, from about 1 ug to about 50 mg per kg of body weight, from about 1 mg to about 500 mg per kg of body weight; and from about 1 mg to about 50 mg per kg of body weight. Alternatively, the amount of steroid and/or vitamin D administered to achieve a therapeutic effective dose is about 0.1 ng, 1 ng, 10 ng, 100 ng, 1 ug, 10 ug, 100 ug, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater. In some embodiements, the pharmaceutical compositions described herein include an amount of neuroprotective steroid or a pharmaceutically acceptable salt, ester or prodrug thereof that is selected from the group consisting of (i) 0.1 mg to 5000 mg, (ii) 0.5 mg to 1000 mg, and (iii) 1 mg to 500 mg. In some embodiments, the neuroprotective steroid, or pharmaceutically acceptable salt, ester or prodrug thereof is administered intravenously at 12 mg/kg per day, for 3-5 days.

In certain embodiments, the compounds described herein are compounded with a suitable pharmaceutically acceptable carrier in a unit dosage form. A unit dosage form, such as a preselected amount of liquid composition, can, for example, contain the compound in amounts ranging from about 5 to about 1000 mg, or from about 250 to about 750 mg. Expressed in proportions, the active compound is generally present in from about 10 to about 750 mg/ml of carrier. Liquid formulations of progesterone can comprise about 1-100 mg/ml of vehicle. In the case of compositions containing supplementary active ingredients, the dosages are determined by reference to the usual dose and manner of administration of the said ingredients.

The active ingredients can exhibit activity, particularly in treatment or prevention of secondary reactions from brain injuries such as TBI or stroke when administered in amounts ranging from about 0.1 mg to about 100 mg per kilogram of body weight per day. A preferred dosage regimen for optimum results would be from about 0.5 mg to about 50 mg per kilogram of body weight per day, and such dosage units are employed that a total of from about 0.25 gram to about 3.0 grams of the active compound for a subject of about 70 kg of body weight are administered in a 24-hour period. This dosage regimen may be adjusted to provide the optimum therapeutic response and can be administered one to three times a day in dosages of about 600 mg per administration. For example, several divided doses may be administered daily or the dose may be proportionally reduced as indicated by the exigencies of the therapeutic situation.

In one embodiment of the present invention, the neuroprotectivc steroid is administered once or several times a day. The duration of the treatment may be once per day for a period of about 1, 2, 3, 4, 5, 6, 7 days or more. The daily dose can be administered either by a single dose in the form of an individual dosage unit or several smaller dosage units or by multiple administration of subdivided dosages at certain intervals. For instance, a dosage unit can be administered from about 0 hours to about 1 hr, about 1 hr to about 24 hr, about 1 to about 72 hours, about 1 to about 120 hours, or about 24 hours to at least about 120 hours post injury. Alternatively, the dosage unit can be administered from about 0.5, 1, 2, 3, 4, 5, 6, 7, 8, 9, 10, 11, 12, 13, 14, 15, 16, 17, 18, 19, 20, 21, 22, 23, 24, 30, 40, 48, 72, 96, 120 hours or longer post injury. The duration of the constant dosing regimen is about 12, 24, 36, 60, 72, 84, or 120 hours or about 1 to 24 hours, about 12 to 36 hours, about 24 to 48 hours, about 36 to 60 hours, about 48 to 72 hours, about 60 to 96 hours, about 72 to 108 hours, about 96 to 120 hours, or about 108 to 136 hours. Subsequent dosage units can be administered any time following the initial administration such that a therapeutic effect is achieved. For instance, additional dosage units can be administered to protect the subject from the secondary wave of edema that may occur over the first several days post-injury. In specific embodiments, the subject undergoing the therapy with is administered a constant neuroprotective steroid dosing regimen. By “constant dosing regimen” is intended the subject undergoing therapy is administered a constant total hourly infusion dose over the course of treatment.

Administration of the compositions of the invention may be performed by many methods known in the art. The present invention comprises all forms of dose administration including, but not limited to, systemic injection, parenteral administration, intravenous, intraperitoneal, intramuscular, transdermal, buccal, subcutaneous and intracerebroventricular administration. Alternatively, the neuroprotective steroid or vitamin D may be administered directly into the brain or cerebrospinal fluid by any intracerebroventricular technique including, for example, lateral cerebro-ventricular injection, lumbar puncture or a surgically inserted shunt into the cerebro ventricle of a patient. Methods of administering may be by dose or by control release vehicles.

If administered intravenously, carriers include physiological saline or phosphate buffered saline (PBS).

While the methods of the invention are not bound by any theory, it is believed that a traumatic CNS injury, may make the blood/brain barrier more permeable allowing entry of large molecules that would not normally cross the blood/brain barrier to enter the cerebral spinal fluid. For examples of intravenous, intraperitoneal, intramuscular, and subcutaneous administration of neurotrophic agents to treat CNS injuries sec, for example, U.S. Pat. No. 5,733,871 and WO 97/21449, both of which are herein incorporated by reference.

Additional pharmaceutical methods may be employed to control the duration of action. Controlled release preparations may be achieved by the use of polymers to complex or absorb the compounds. The controlled delivery may be exercised by selecting appropriate macromolecules (for example, polyesters, polyamino acids, polyvinyl pyrrolidone, ethylene-vinylacetate, methylcellulose, carboxymethylcellulose, or protamine sulfate). The rate of drug release may also be controlled by altering the concentration of such macromolecules.

Another possible method for controlling the duration of action comprises incorporating the therapeutic agents into particles of a polymeric substance such as polyesters, polyamino acids, hydrogels, poly(lactic acid) or ethylene vinylacetate copolymers. Alternatively, it is possible to entrap the therapeutic agents in microcapsules prepared, for example, by coacervation techniques or by interfacial polymerization, for example, by the use of hydroxymethyl cellulose or gelatin-microcapsules or poly(methylmethacrylate) microcapsules, respectively, or in a colloid drug delivery system, for example, liposomes, albumin, microspheres, microemulsions, nanoparticles, nanocapsules, or in macroemulsions. Such teachings are disclosed in Remington's Pharmaceutical Sciences (1980). Ideally the compounds should be administered to achieve peak plasma concentrations of the active compound of from about 0.2 to 70 μM, preferably about 1.0 to 10 μM. This may be achieved, for example, by the intravenous injection of an appropriate concentration of the active ingredient, optionally in saline, or administered as a bolus of the active ingredient.

The concentration of the compounds in the drug composition will depend on absorption, inactivation and excretion rates of the extract as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The compounds may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The compounds can be administered as a component of an elixir, suspension, syrup, wafer, chewing gum or the like. A syrup may contain, in addition to the active compounds, sucrose as a sweetening agent and certain preservatives, dyes and colorings and flavors. The compounds can also be mixed with other active materials that do not impair the desired action, or with materials that supplement the desired action, such as antibiotics, antifungals, anti-inflammatories, or other anti-autoimmune compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

Formulations suitable for parental administration conveniently comprise a sterile aqueous preparation of the active compound, which can be isotonic with the blood of the recipient.

Nasal spray formulations comprise purified aqueous solutions of the active agent with preservative agents and isotonic agents. Such formulations are preferably adjusted to a pH and isotonic state compatible with the nasal mucous membranes.

Formulations for rectal administration may be presented as a suppository with a suitable carrier such as cocoa butter, or hydrogenated fats or hydrogenated fatty carboxylic acids.

Ophthalmic formulations are prepared by a similar method to the nasal spray, except that the pH and isotonic factors are preferably adjusted to match that of the eye.

Topical formulations comprise the active compound dissolved or suspended in one or more media such as mineral oil, petroleum, polyhydroxy alcohols or other bases used for topical formulations. The addition of other accessory ingredients as noted above may be desirable.

Further, the present invention provides liposomal formulations of the compounds, particularly of the neuroprotective steroid compounds, salts, esters and prodrugs thereof. The technology for forming liposomal suspensions is well known in the art. When the compounds or salts thereof are an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer that forms the structure of the liposome. In either instance, the liposomes that are produced may be reduced in size, as through the use of standard sonication and homogenization techniques. The liposomal formulations containing the progesterone analogue or salts thereof, may be lyophilized to produce a lyophilizate which may be reconstituted with a pharmaceutically acceptable carrier, such as water, to regenerate a liposomal suspension.

Pharmaceutical formulations are also provided which are suitable for administration as an aerosol, by inhalation. These formulations comprise a solution or suspension of the compound or a salt thereof or a plurality of solid particles of the compound or salt. The desired formulation may be placed in a small chamber and nebulized. Nebulization may be accomplished by compressed air or by ultrasonic energy to form a plurality of liquid droplets or solid particles comprising the compounds or salts.

In another embodiment, the compounds are prepared with carriers that will protect them against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid. Methods for preparation of such formulations will be apparent to those skilled in the art.

### IX. SYNTHESIS OF CERTAIN STEROID ANALOGS

Schemes 1-8 below describe the preparation of selected steroid analogues. It is understood that the specific synthetic steps are not limited to the reactions shown in the schemes and that many alternative reaction sequences known in the art are suitable for the preparation of the analogues. Furthermore, it is understood that any naturally occurring or synthetic amino acid in the D, L or D,L configuration may be used. The invention is not limited by the type of protecting group and any suitable protecting group may be used. Protecting groups for amino agroups and ketone groups are well known in the art and described by Greene et al. Protective Groups in Organic Synthesis, John Wiley and Sons, Third Edition, 1999.

### Progesterone Analogues Substituted at the 3-Position:

Compounds derivatized at the 3-position of the steroid ring system to comprise an ester of an amino acid may be prepared using the general process described in Scheme I below. Starting from progesterone, the carbonyl group at the 3-position is selectively reduced to produce the allylic alcohol 2. One example of a selective reduction is the Luche Reduction which use sodium borohydride and cerium trichloride in methanol (see Luche, J.-L. J. Am. Chem. Soc., 1978, 100, 2226). Alcohol 2 is then esterified with a suitable amino acid derivative form the progesterone analogue 3. The protecting group is removed and a suitable salt, such as an HCl salt, may be formed, if desired.

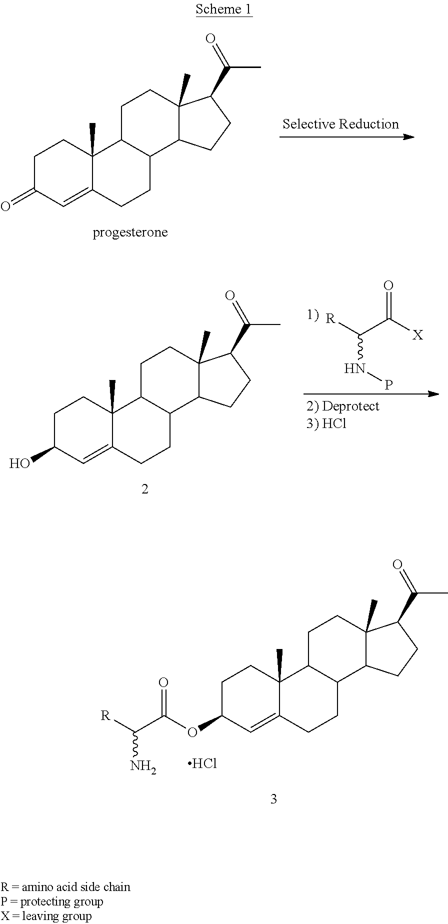

In one embodiment, the ester bond may be formed by reaction of the hydroxyl group of 2 with a protected amino acid acyl halide, where X is chloro, bromo, iodo or fluoro. In another embodiment, the ester bond may be formed by reacting the hydroxyl group with an activated carboxylic acid, where X is an activated leaving group. Many reagents are known that will activate carboxyl groups to react with nucleophiles. For example, a variety of peptide coupling reagents well known in the art are used to activate carboxyl groups in-situ to react with amino groups of amino acids to form peptide bonds. These reagents can also activate carboxylic acids to form reactive intermediates that will react with hydroxy groups on the steroid compound. Non-limiting examples of the carboxyl activating groups include carbodiimide reagents, phosphonium reagents such as benzotriazolyloxy-tris-(dimethylamino) phosphonium hexafluorophosphate (BOP) and the like, uronium or carbonium reagents such as O-(benzotriazol-1-yl)-N,N,N′,N′-tetramethyluronium hexafluorophosphatc (HBTU), benzotri azol-1-yl-oxy-tripyrrolidinophosphoni um hexafluorophosphate (PyBOP) and the like; 1-ethoxycarbonyl-2-ethoxy-1,2-dihydroqunoline (EEDQ); 1-methyl-2-chloropyridinium iodide (Muikaiyama's reagent) and the like. In other embodiments, the ester may be formed by trans-esterification of another ester group including active esters such as a p-nitrophenyl ester, a pentafluorophenylester, an N-hydroxysuccinimidyl ester, a 1-hydroxybenzotriazolyl ester and the like. An acyl azide group may also be used to form the ester bond. In another embodiment, the ester may also be formed by reaction of the hydroxy with a symmetric or mixed anhydride (X is RC(O)O—). Catalysts such as 4-dimethylaminopyridine (DMAP) and the like may be used to facilitate the ester formation.

### Progesterone Analogues Substituted at the 20-Position:

Scheme 2 below illustrates the general synthetic process for the formation of steroid analogues that comprise amino acid residues at the 20-position of the ring. In this process, progesterone is reduced to the diol using a strong reducing reagent, such as lithium aluminum hydride. The allylic hydroxyl group is then selectively oxidized to produce the enone 4, with the C-20 hydroxyl group intact. Any suitable oxidizing agent that will selectively oxidize an allylic alcohol may be used. One non-limiting example is manganese dioxide (MnO₂). The resulting alcohol 3 is then cstcrified to produce the desired steroid analogue 5 comprising an amino acid residue at the 20-position. As described above for Scheme 1, the esterification reaction may be accomplished with a variety of reagents, including a protected amino acid halide or with a protected amino acid using a coupling reagent known in the art to activate carboxylate groups.

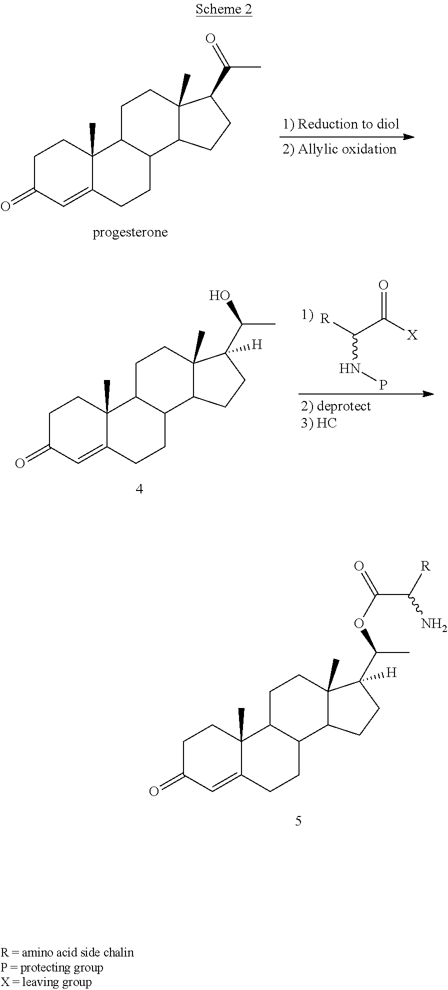

### C-3 Progesterone C═NR Derivatives

Scheme 3 below illustrates the preparation of an analogue of Formula VII, substituted at C-3 with the group ═N—R³ where R³ is —OR¹¹, and R¹¹ is an amino acid residue. The C20 carbonyl is first protected with a suitable protecting group such as the cyclic ketal 6 to prevent reaction with the nucleophilic hydroxylamine. The remaining enone is reacted with hydroxylamine to produce a mixture of E/Z 7a and 7b. The E-oxime 7a is then esterified with a suitably protected amino acid halide or using a protected amino acid with a coupling reagent as described above for Scheme 1 to produce the E-isomer of protected analogue 8a. Removal of the cyclic acetal under acidic conditions followed by removal of the amino protecting group under typically basic conditions provides the C3 analogue 9a, which is converted to the hydrochloride salt upon treatment with HCl. The corresponding Z-isomers 8b and 9b are prepared using Z-oxime 7b in the same manner.

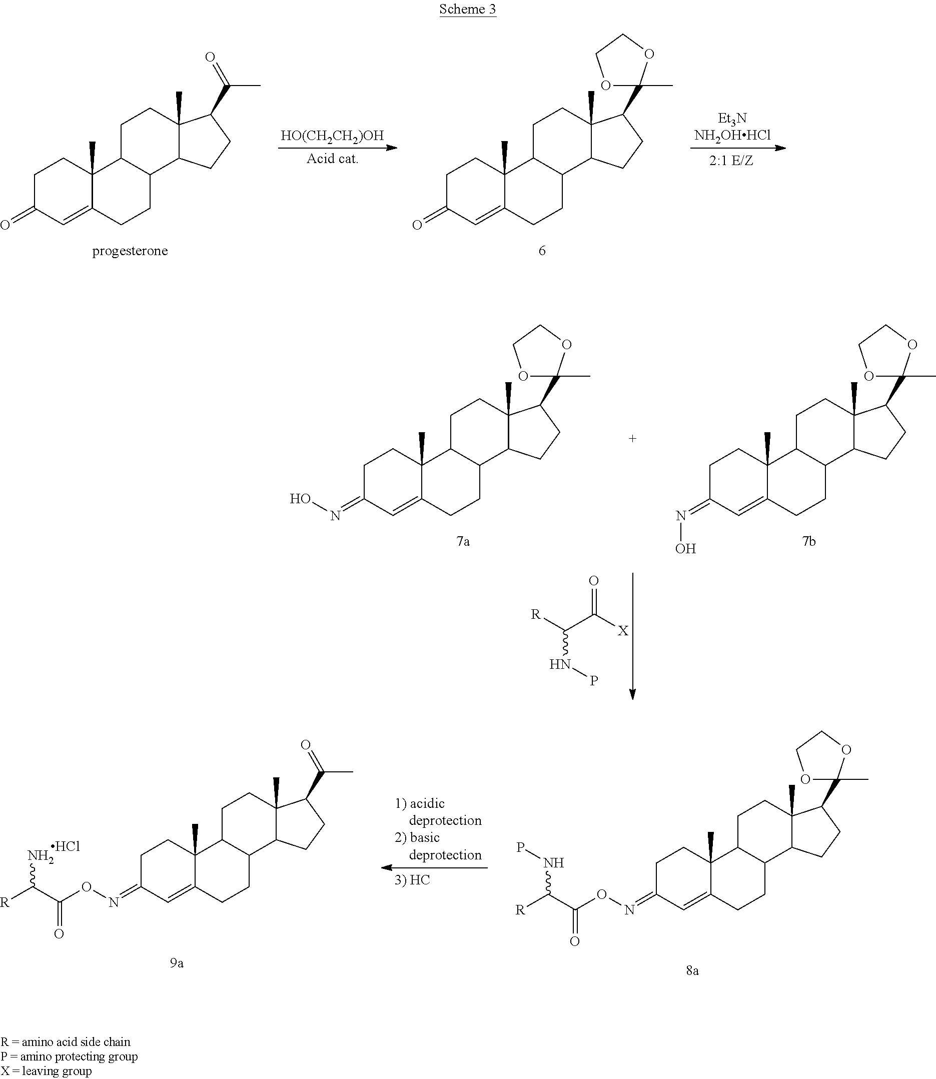

Scheme 4 shows the general synthesis of an analogue of Formula IV substituted at C-3 with the group R⁵—N═C where R⁵ is R—NH—, and R is an amino acid residue. Starting from protected intermediate 6, the C₁₋₃ carbonyl is reacted with hydrazine to produce the hydrazone 10. The hydrazone is then reacted with a suitable reactive amino acid derivative as described above for Scheme 1 to yield the hydrazide 11. The hydrazide may be converted to a pharmaceutically acceptable salt by treatment with a pharmaceutically acceptable acid, such as HCl.

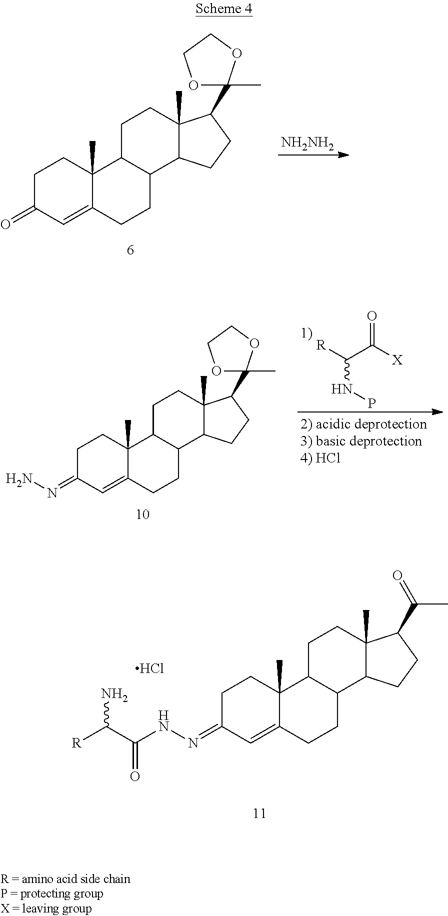

### C-3 Pregnanolone and Allopregnanolone Derivatives

Scheme 5 below shows the preparation of allopregnanolone analogues substituted at C-3 with an amino acid residue. Pregnenolonc is first reduced with hydrogen catalyzed by palladium on carbon to produce compound 12 in the 3-beta, 5-alpha configuration. Compound 12 is then esterified as described for Scheme 1 above with a reactive protected amino acid reagent followed by deprotection to produce compound 13 substituted at the C-3 position with an amino acid residue. The HCl salt is formed by treatment with HCl as before.

To produce compound 15, in which the amino acid substituent has the opposite stereoisomeric configuration at C-3, the stereo configuration of the hydroxyl group in compound 12 is inverted using Mitsunobu conditions (see Mitsunobu et al., Bull. Chem. Soc. Japan 1967, 40, 2380-2382 and Mitsunobu et al., Synthesis 1981, 1-28 and Castro et al., Org. React. 1983, 29, 1) to form compound 14 with the 3-alpha, 5-alpha configuration. Compound 14 is cstcrified as described above to produce compound 15 with the 3-alpha, 5-alpha configuration, followed by treatment with HCl to form the salt.

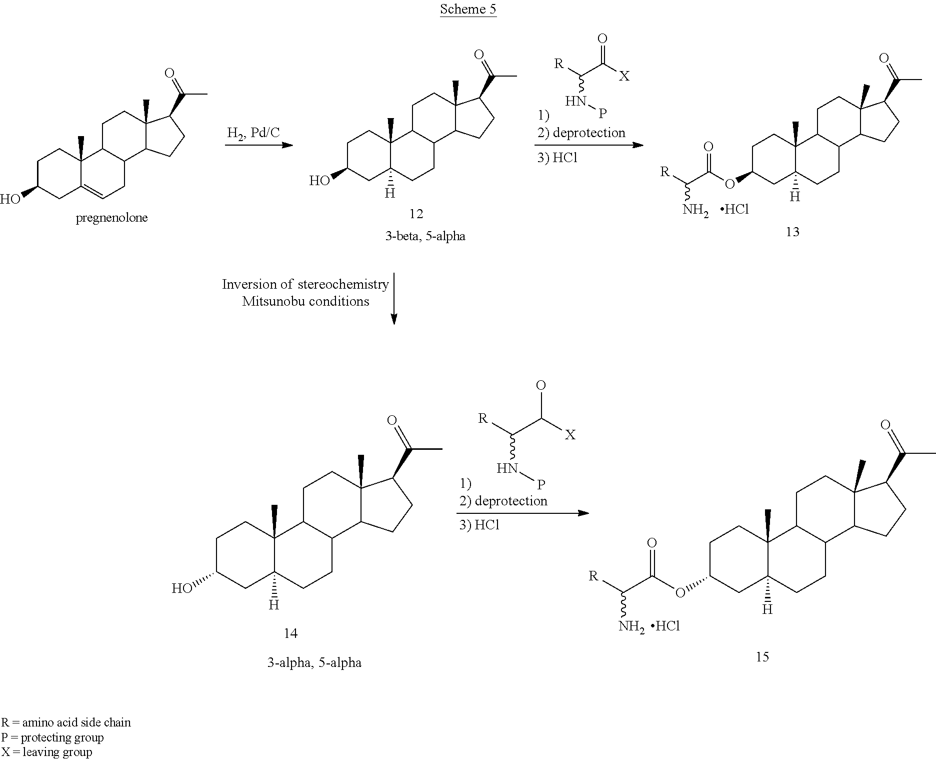

Scheme 6 below shows a general process for the preparation of C-3 substituted pregnanolone analogues in different stereoisomeric configurations. Starting from progesterone, reduction of the enone with hydrogen under palladium on carbon forms compound 16. Reduction of the cyclic ketone, using a suitable reducing agent such as sodium borohydride, forms a mixture of alcohols 17a and 17b. Esterification of alcohols 17a and 17b followed by removal of the protecting group and salt formation provides pregnanolone analogues 18a and 18b.

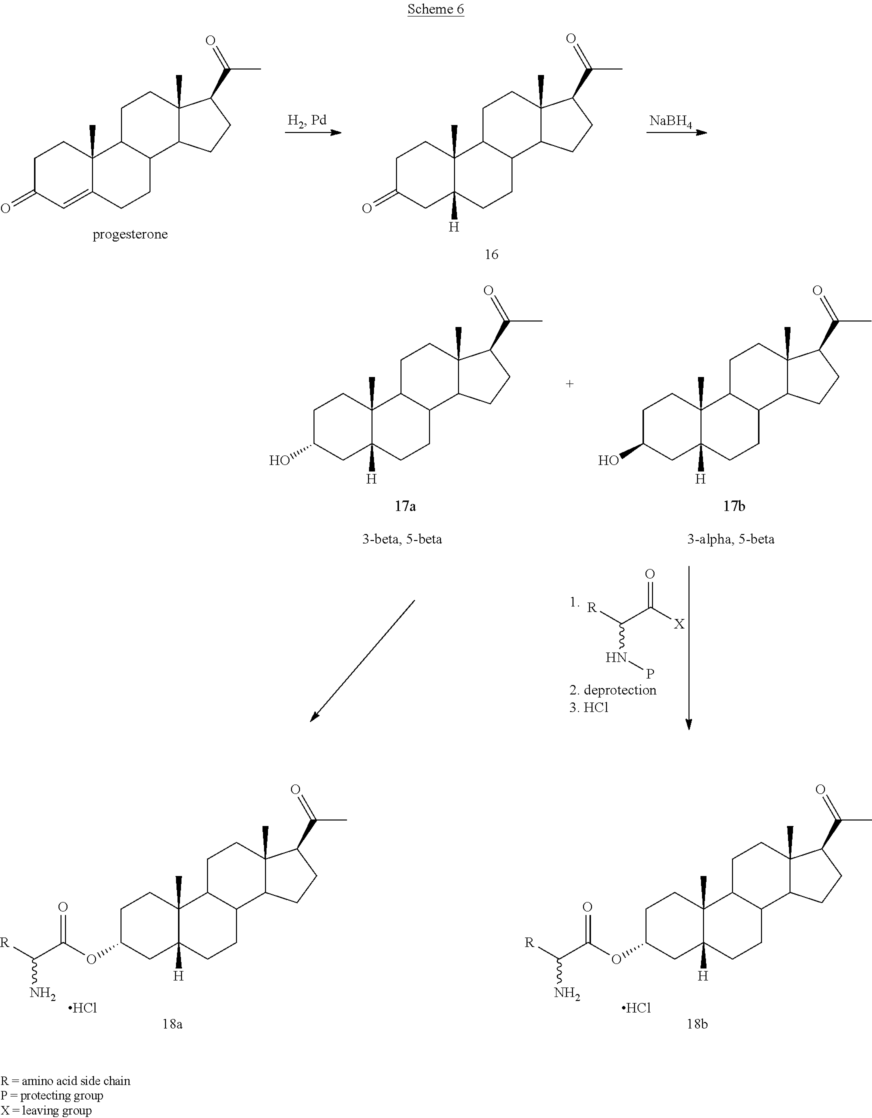

Steroid analogues with a double bond between the C5 and C6 positions may be prepared according to the general process shown in Scheme 7 below.

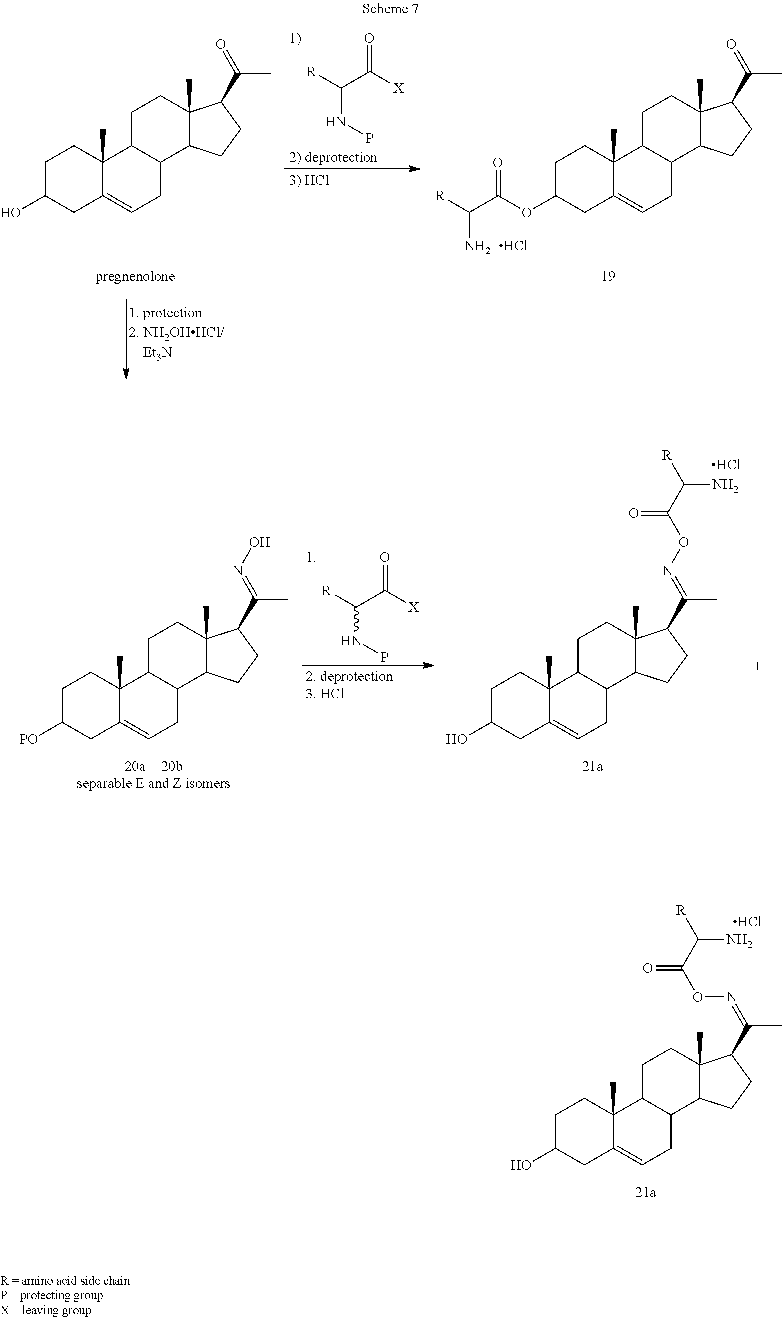

Esterification of pregnenolone with a suitably protected amino acid as described for scheme 1 above provides compound 19, with an amino acid residue at the 3-position. Protection of the hydroxyl of pregnenolone followed by reaction with hydroxylamine provides the E- and Z-isomers 20a and 20 b. If desired, the isomers may be separated at this stage. Reaction of 20a and 20b with a suitably protected amino acid, followed by deprotection and treatment with HCl provides compounds 21a and 21b.

Modification of the process shown in Scheme 7 leads to related compounds with a double bond between C5 and C6. For example, to obtain the steroid analogues corresponding to compounds 21a and 21b in which the C-3 hydroxyl is in the ketone oxidation state, compounds 20a and 20b may be deprotected to the alcohol and oxidized to form the ketone prior to reaction with the activated amino acids reagent. Reduction of compound 19 will provide the corresponding C20 alcohol, which may be esterified as described above to form an analogue substituted at both C3 and C20 positions.

Reduction of protected pregnenolone followed by esterification of the resulting C20 hydroxyl group with a suitably protected activated amino acid will provide the C20 amino acid substituted derivative after removal of the protecting group.

Protection of the C20 ketone, for example as a cyclic ketal, followed by oxidation of the C3 hydroxyl to the corresponding ketone and then reaction with hydroxylamine will provide the corresponding C3 oximes, which can be reacted with suitably protected activated amino acids to prepare the ═N—NR¹¹R¹² compounds.

Analogues with a double bond between the C1 and C2 carbons may be prepared according to the process depicted in Scheme 8 below.

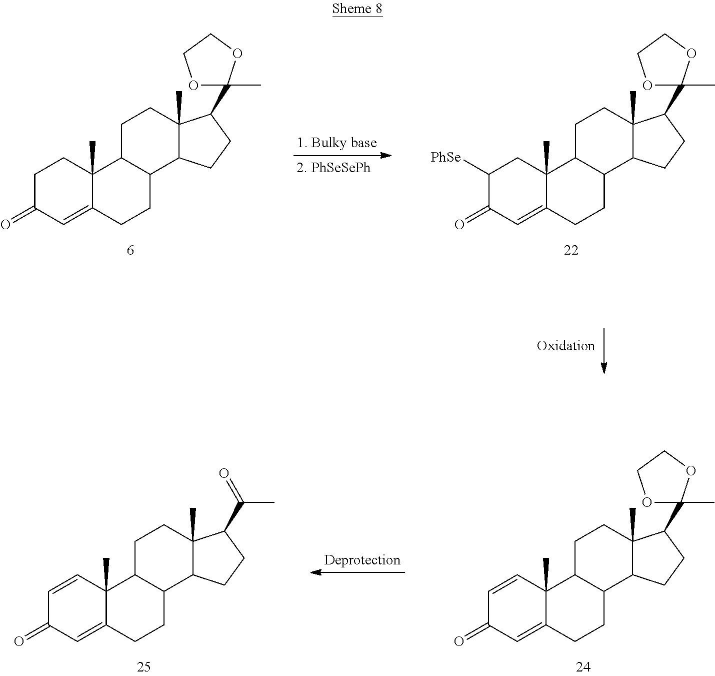

Starting from protected compound 6, treatment with a bulky base such as lithium diisopropylamide (LDA) or the like, to form the enolate species, followed by reaction with a suitable source of electrophilic selenium, such as diphenyldiselenide, provides compound 22. Treatment of compound 22 with a suitable oxidizing agent, such as hydrogen peroxide, provides compound 24, which is deprotected to provide compound 25:

### Enantiomeric Progesterone Compounds

In one embodiment, the invention provides enantiomeric progesterone and neuroprotective steroid compounds. The enantiomer of progesterone (ent-PROG) has shown similar efficacy to progesterone and allopregnanolone across several measures relevant to ncuroprotection, including the reduction of cerebral edema, reduction of pro-inflammatory cytokinc expression, and reduction in proapoptotic p53 protein expression. Ent-PROG treatment was also shown to result in significantly increased glutathione reductase activity, a measure of its potential to minimize oxidative stress following TBI, relative to both progesterone and allopregnanolone. Although it binds with moderate affinity to the classical progesterone receptor (PR), ent-PROG does not activate PR-mediated gene transcription. Thus it is thought that ent-PROG is able to achieve its neuroprotective effects either through transcription-independent PR-mediated signaling or via PR-independent pathways. In light of these findings, and with the goal of providing a compound of improved efficacy relative to PROG or allopregnanolone, the development of a complementary set of ent-PROG based analogue compounds was pursued.

The synthesis of ent-PROG closely followed the methods previously described for thepreparation of 19-nor˜teroids'a˜s˜well as the later extensions to this work byRychnovsky and co-workers in their application to the total synthesis of ent-cholesterol. Addition of methyl vinyl ketone (MVK) to 2-methyl-1,3-cyclopentadione (37, Scheme 9) gave the trikctone 38. The organocatalyst D-proline was then used inorder to achieve asymmetric cyclization of 38 to give the Hajos-Parrish ketone (39). Sodium borohydride reduction of 39 was followed by protection of the newly formedsecondary alcohol 40 as its tevt-butyl ether (41). Introduction of an α-methylene groupwas achieved through initial carbonation of 41 with Stiles' reagent, methyl magncsiumcarbonatc (MMC), in DMF. Selective reduction of the C-4-C-5 double bond ofcompound 43 was immediately followed by decarboxylation of the unstable saturatedintermediate 44 to give the enone 45 with trans ring junction.

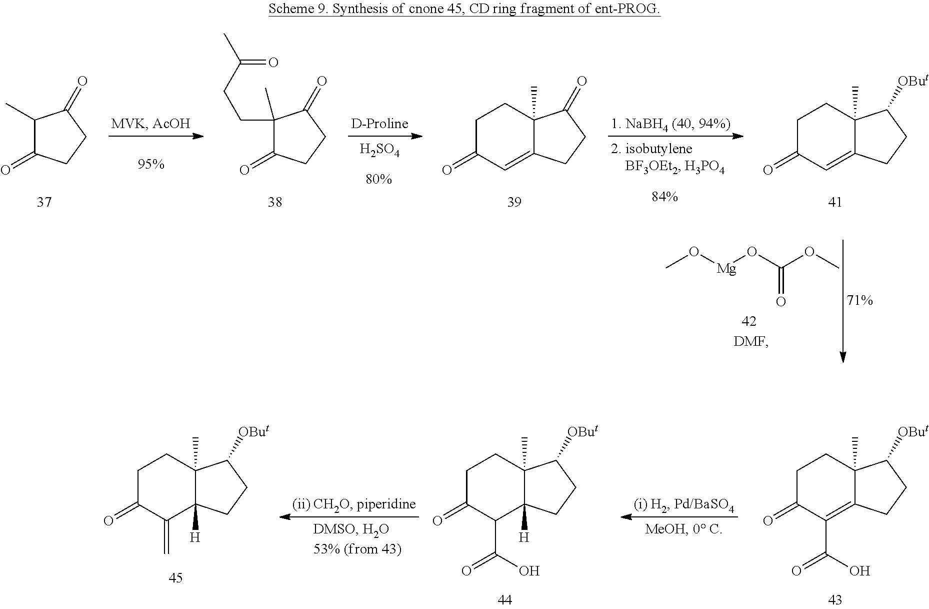

Synthesis of the β-keto ester annulating agent 50 began with ketalization of ethyl-5-oxohexanoate and subsequent reduction of the ester 47 with LiAlH₄ to give alcohol 48 (Scheme 10). Swern oxidation of 48 was followed by tin(11) chloride catalyzed coupling with ethyl diazoacetate to give the P-keto ester 50.

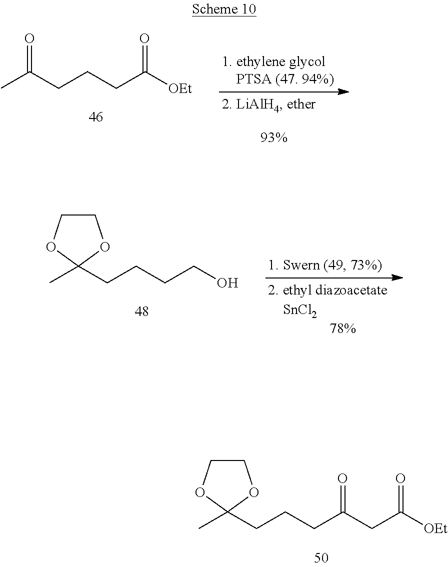

Michael addition of 6-keto ester 50 to enone 45, along with in situ Robinson annulation, saponification, and finally decarboxylation, gave the BCD ring system 51. Reductive alkylation served to introduce what would become the C-19 methyl group of ent-FROG. Reflux of 52 overnight in methanolic HCl gave ent-testosterone (53). Ent-testosterone was then prepared as the C-3 ketal 54 and the C-17 secondary alcohol was oxidized using pyridinium chlorochromate (PCC) to give ketone 55. Treatment of 55 with ethyltriphenylphosphonium bromide under Wittig conditions gave the alkene 56. A final three step sequence involving hydroboration, oxidation, and acid catalyzed removal of the ketal was carried out without intermediate purification steps to give ent-PROG (57) in good yield.

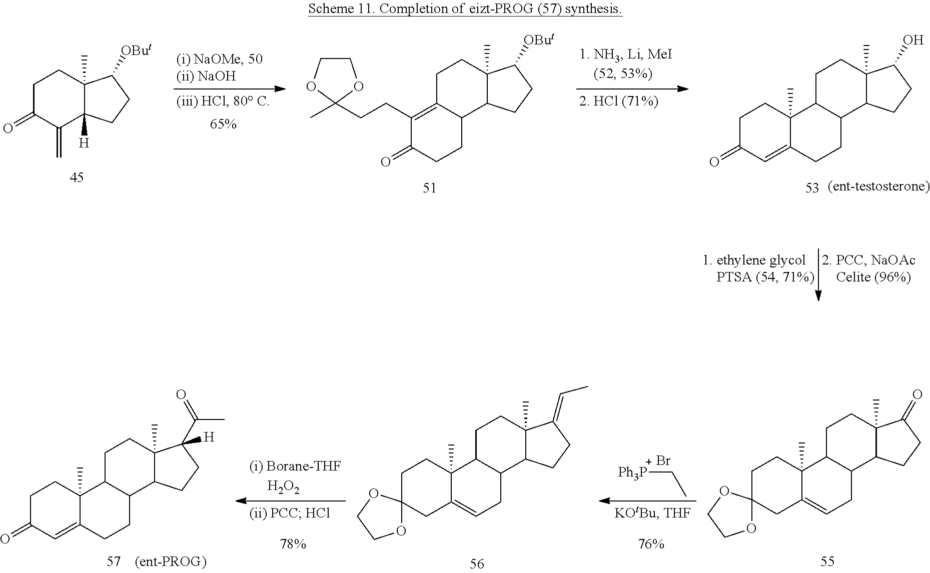

Luche reduction of ent-PROG gave the C-3 a-hydroxy compound 58 (Scheme 12). The same series of reactions involving amino acid coupling, Fmoc cleavage, and HCl salt formation that had been developed for the C-3 nut-PROG series of compounds was applied here to give the ent-PROG derivative P2-13. Additional neuroprotective analogues derived from ent-PROG are prepared according to the description provided above and in the following examples.

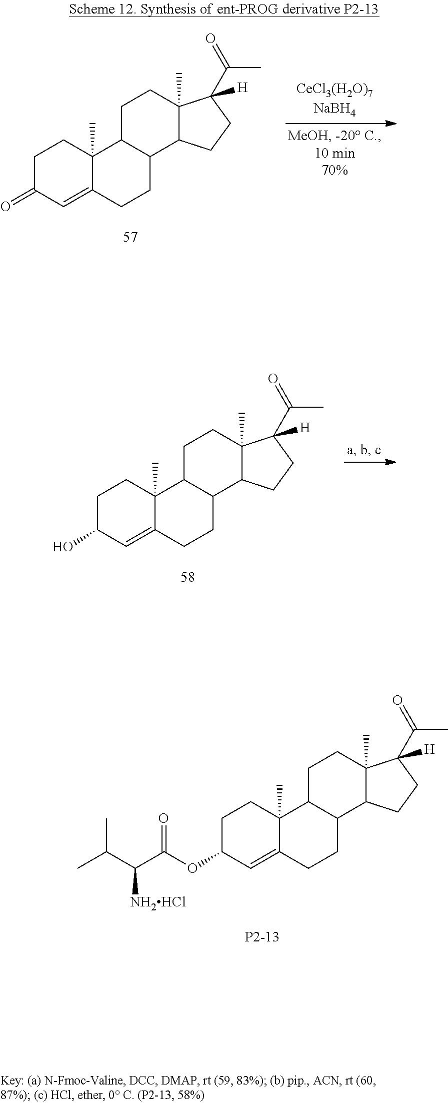

The present invention will be understood more readily by reference to the following examples, which are provided by way of illustration and are not intended to be limiting of the invention.

### X. ILLUSTRATIVE EMBODIMENTS

The following embodiments are illustrative only.

In accordance with the composition embodiments, there are provided pharmaceutical composition comprising (a) a neuroprotective steroid or a pharmaceutically acceptable salt, ester or prodrug thereof and (b) vitamin D, optionally in a pharmaceutically acceptable carrier. In any of the embodiments described herein, the neuroprotective steroid is selected from the group consisting of progesterone and allopregnanolone. In other embodiments, the neuroprotective steroid is represented by formula (I):

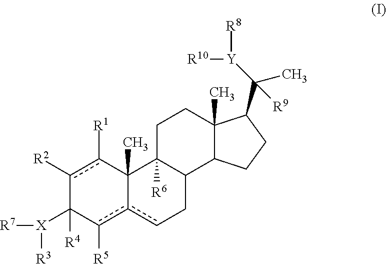

wherein X is O, N or S;

Y is O,N or S;

R¹, R², R⁵ and R⁶ are independently hydrogen, alkyl, halogen, hydroxylcycloalkyl, cycloalkenyl, alkenyl, alkynyl, aryl, alkylaryl, arylalkyl, heterocyclic, heteroaryl, amino, thiol, alkoxy, sulfide, nitro, cyano, azide, sulfonyl, acyl, carboxyl, an ester, an amide, carbamate, carbonate, an amino acid residue or a carbohydrate;

R⁴ is hydrogen or alkyl; or R⁴ and R⁷ together form a double bond;

R³ is hydrogen, optionally substituted acyl, a residue of an amino acid, a carbohydrate, —OR¹¹, —NR¹¹R¹² or R³ is absent;

R⁷ is hydrogen or is absent, or R⁷ together with R⁴ forms a double bond;

R⁸ is hydrogen, optionally substituted acyl, a residue of an amino acid, a carbohydrate, —OR¹¹, —NR¹¹R¹² or R⁸ absent;

R⁹ is hydrogen or alkyl; or R⁹ and R¹⁰ together form a double bond;

R¹⁰ is hydrogen or is absent, or R¹⁰ together with R⁹ forms a double bond;

R¹¹ is the residue of an amino acid, a carbohydrate or an optionally substituted ester or a substituted acyl;

R¹² is hydrogen or alkyl; and

the dotted line indicates the presence of either a single bond or a double bond, wherein the valences of a single bond are completed by hydrogens,

provided that

at least one of XR³R⁷ or YR⁸R¹⁰ is not ═O or OH, and that if the dotted line between C4 and C5 or between C5 and C6 represents a double bond then the other dotted line between C4 and C5 or between C5 and C6 represents a single bond; and with the proviso that neither XR³R⁷ nor YR⁸R¹⁰ represent an ester of aspartic acid, glutamic acid, gama amino butyric acid or a-2-(hydroxyethylamino)-propionic acid; and

with the proviso that when Y is N, R⁸ does not represent aspartic acid, glutamic acid, gama amino butyric acid or a-2-(hydroxyethylamino)-propionic acid.

In other embodiments, the neuroprotective steroid is represented by any one of formulas (II)-(XII) described herein above.

In any of the embodiments described herein, the vitamin D may be selected from the group consisting of ergocalciferol, cholecalciferol, calcitriol, seocalcitol, doxercalciferol and calcipotriene, and in specific embodiments comprises a 1,25-dihydroxyvitamin D₃ (1,25-diOH-D).

In any of the embodiments described herein, the composition may comprise an amount of vitamin D selected from the group consisting of (i) at least 1000 international units (IU), (ii) at least 1500 IU, (iii) at least 2000 IU, (iv) at least 2500 IU, (v) at least 3000 IU, (vi) at least 3500 IU, (vii) at least 4000 IU, (viii) at least 5000 IU, (ix) at least 10,000 IU, (x) at least 25,000 IU, and (xi) at least 50,000 IU.

In any of the embodiments described herein, the composition may comprise an amount of neuroprotective steroid or a pharmaceutically acceptable salt, ester or prodrug thereof selected from the group consisting of (i) 0.1 mg to 5000 mg, (ii) 0.5 mg to 1000 mg, and (iii) 1 mg to 500 mg.

In any of the embodiments described herein, the composition may be formulated for oral, nasal, intravenous, or intramuscular administration.Also within the scope of the invention is the use of any composition as described herein, for treating or preventing nervous system injury or other condition discussed herein in a patient in need thereof.

In accordance with other embodiments, methods of treating or preventing nervous system injury in a patient in need thereof are provided. In general, the methods comprise administering to said patient (i) a neuroprotective steroid or a pharmaceutically acceptable salt, ester or prodrug thereof, and (ii) vitamin D.

In any of the embodiments described herein, the neuroprotective steroid may comprise, or alternatively consist of, progesterone or allopregnanolone. In other embodiments, the neuroprotective steroid is represented by formula (I):

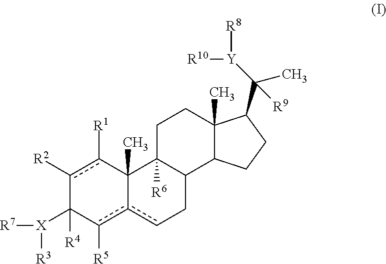

wherein X is O, N or S;

Y is O, N or S;

R¹, R², R⁵ and R⁶ are independently hydrogen, alkyl, halogen, hydroxylcycloalkyl, cycloalkenyl, alkenyl, alkynyl, aryl, alkylaryl, arylalkyl, heterocyclic, heteroaryl, amino, thiol, alkoxy, sulfide, nitro, cyano, azide, sulfonyl, acyl, carboxyl, an ester, an amide, carbamate, carbonate, an amino acid residue or a carbohydrate;

R⁴ is hydrogen or alkyl; or R⁴ and R⁷ together form a double bond;

R³ is hydrogen, optionally substituted acyl, a residue of an amino acid, a carbohydrate, —OR¹¹, —NR¹¹R¹² or R³ is absent;

R⁷ is hydrogen or is absent, or R⁷ together with R⁴ forms a double bond;

R⁸ is hydrogen, optionally substituted acyl, a residue of an amino acid, a carbohydrate, —OR¹¹, —NR¹¹R¹² or R⁸ absent;

R⁹ is hydrogen or alkyl; or R⁹ and R¹⁰ together form a double bond;

R¹⁰ is hydrogen or is absent, or R¹⁰ together with R⁹ forms a double bond;

R¹¹ is the residue of an amino acid, a carbohydrate or an optionally substituted ester or a substituted acyl;

R¹² is hydrogen or alkyl; and

the dotted line indicates the presence of either a single bond or a double bond, wherein the valences of a single bond are completed by hydrogens,

provided that

at least one of XR³R⁷ or YR⁸R¹⁰ is not ═O or OII, and that if the dotted line between C4 and C5 or between CS and C6 represents a double bond then the other dotted line between C4 and C5 or between C5 and C6 represents a single bond; and with the proviso that neither XR³R⁷ nor YR⁸R¹⁰ represent an ester of aspartic acid, glutamic acid, gama amino butyric acid or a-2-(hydroxyethylamino)-propionic acid; and

with the proviso that when Y is N, R⁸ does not represent aspartic acid, glutamic acid, gama amino butyric acid or a-2-(hydroxyethylamino)-propionic acid.

In other embodiments, the neuroprotective steroid is represented by any one of formulas (II)-(XII) described herein above.

In any of the embodiments described herein, the vitamin D may comprise, or alternatively consist of, ergocalciferol, cholecalciferol, calcitriol, seocalcitol, doxercalciferol and/or calcipotriene, and/or 1,25-dihydroxyvitamin D₃ (1,25-diOH-D). In any of the embodiments described herein, the methods may involve administering the neuroprotective steroid and vitamin D in the same composition or in different compositions, at the same time or at different times, by the same route or by different routes.

In any of the embodiments described herein, the methods may comprise administering an amount of vitamin D that is selected from the group consisting of (i) at least 1000 international units (IU), (ii) at least 1500 IU, (iii) at least or at least 2000 IU, (iv) at least 2500 IU, (v) at least 3000 IU, (vi) at least 3500 IU, and (vii) least 4000 IU, (viii) at least 5000 IU, (ix) at least 10,000 IU, (x) at least 25,000 IU, and (xi) at least 50,000 IU or greater.

In any of the embodiments described herein, the methods may comprise administering an amount of the neuroprotective steroid or pharmaceutically acceptable salt, ester or prodrug thereof that is selected from the group consisting of (i) 0.1 mg to 5000 mg, (ii) 0.5 mg to 1000 mg, and (iii) 1 mg to 500 mg, or (i) 0.001 mg/kg/day to 1000 mg/kg/day, (ii) 0.05 mg/kg/day to 500 mg/kg/day, and (iii) 0.1 mg/kg/day to 300 mg/kg/day.

In any of the embodiments described herein, the neuroprotective steroid or pharmaceutically acceptable salt, ester or prodrug thereof may be administered orally, nasally, intravaneously, or intramuscularly, as may the vitamin D.

In any of the embodiments described herein, the methods may commence at a time selected from the group consisting of (i) one day from the nervous system injury; (ii) less than one day from the nervous system injury; (iii) less than 18 hours from the nervous system injury; (iv) less than 12 hours from the nervous system injury; and (v) less than six hours from the nervous system injury.

In any of the embodiments described herein, the nervous system injury to be treated or prevented may be selected from neurodegenerative reactions to injury or disease, traumatic brain injury, ischemic CNS injury, hemorrhagic CNS injury, spinal cord injury, ischemic stroke, hemorrhagic stroke and anterior optic nerve ischemic injury.

In any of the embodiments described herein, the methods may achieve one or more effects such as (i) reduced neurodegeneration due to apoptosis; (ii) enhanced motor function, (iii) reduced loss of motor function, (iv) reduced inflammation, (v) reduced loss of visual function, and (vi) reduced damage from an inflammatory process.

In any of the embodiments described herein, the patient may be suffering from a vitamin D deficiency or insuffuciency. For example, in some embodiments, the patient has a blood serum level of 25-hydroxy-vitamin D (25-OH-D) selected from the group consisting of (i) less than 20 ng/ml, (ii) less than 15 ng/ml, and (iii) less than 12 ng/ml. In specific embodiments, the vitamin D is administered in an amount effective to reverse the vitamin D deficiency or insuffuciency in said patient. In some embodiments, the patient is at least 60 years old.

In accordance with other embodiments, methods are provided that include (A) assessing the risk of vitamin D deficiency in the patient, and (B) administering to said patient: (i) a neuroprotective steroid or a pharmaceutically acceptable salt, ester or prodrug thereof, and (ii) if said patient is determined to suffer from or be at risk of vitamin D deficiency, vitamin D. In some embodiments, a risk of vitamin D deficiency is determined by the blood serum level of 25-hydroxy-vitamin D (25-OH-D) of the patient. For example, in some embodiments, a blood serum level of 25-hydroxy-vitamin D (25-OH-D) in said patient selected from the group consisting of (i) less than 20 ng/ml, (ii) less than 15 ng/ml, and (iii) less than 12 ng/m¹ is indicative of a patient at risk of vitamin D deficiency. In further embodiments, a risk of vitamin D deficiency is determined by the age of the patient being selected from the group consisting of (i) at least 50 years old, (ii) at least 60 years old, and (iii) at least 70 years old.

### EXAMPLES

### Example 1

### Preparation of Steroid Analogs

All reagents were obtained from Aldrich. Reactions requiring anhydrous conditions were performed in oven-dried glassware under dry argon. All solvents used were anhydrous or kept dry over activated 4 Å molecular sieves. Convection was achieved by use of a magnetic stirring bar unless otherwise noted. The following abbreviations may be used: dichloromethane (DCM), diethyl ether (ether), water (DI), hexane (hex), ethyl acetate (ea), dimethylformamide (DMF), acctonitrile (ACN), tetrahydrofuran (THF), round bottomed flask (RBF), hours (h), minutes (min), millimole (mmol), equivalents (eq). Reaction progxess was monitored via thin-layer chromatography (TLC) on pre-coated glass-backed plates (silica gel 60 Å F₂₅₄, 0.25 mm thickness) purchased from EM Science. Flash chromatography was carried out with silica gel 60 Å (230-400 mesh) from Sorbent Technologies. Automated chromatography was performed on an Isco Combiflash Companion. Unless otherwise stated, organic extracts were dried over commercially available magnesium sulfate and the solvents were removed by rotary evaporation. Brine refers to a saturatcd sodium chloride solution. ¹H and ¹³C NMR spectra were recorded on either a 400 MHz Inova spectrometer or 600 MHz Inova spectrometer in deuterated chloroform (CDCl₃) and referenced to the residual solvent peak (¹H δ 7.27 ppm, ¹³C δ 77.23 ppm). Chemical shifts are reported in parts per million (S), and coupling constants are reported in hertz (Hz). The following abbreviations will be used: singlet (s), doublet (d), triplet (t), quartet (q), multiplet (m). Mass spectra were obtained on either a VG 70-S Nier Johnson or JEOL Mass Spectrometer. Elemental analyses were performed by Atlantic Microlab (Norcross, Ga.).

### Example 1a

### C-3 Progesterone Derivatives

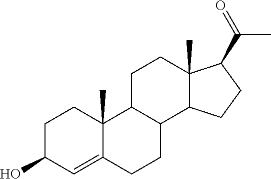

3-β-Hydroxy-progesterone (2). Progesterone (3.14 g, 10.0 mmol) was added with cerium chloride heptahydrate (3.73 g, 10.0 mmol, 1.00 eq) to an oven dried three necked 250 mL RBF with thermometer. Methanol (100 mL) was added under argon and the solution was chilled to −20° C. Sodium borohydride (0.189 g, 5.00 mmol, 0.500 eq) was then added in bulk. Solution temperature raised briefly up to −16° C. After 15 minutes, 37 mL acetone was added and the solution was warmed to ambient temperature. Water (25 mL) was added and the solvent volume was reduced by approximately 100 mL. Ether was added, along with more water, which caused the solution to become clear and colorless. The aqueous layer was extracted with ether. The organic layers were combined, washed with brine, dried, filtered, and concentrated to give 3.14 g white solid. The solid was prepared as a silica cake, loaded onto a 500 mL silica column, and eluted with 3 L 20% ethyl acetate in hexanes, followed by 2 L 25% ethyl acetate in hexanes. Initially eluting pure fractions were combined and concentrated to give 1.56 g white solid that was 90% pure as determined by proton NMR (other 10% was progesterone). (44%) white solid; R_{f}=0.38 (1:1 EA/hex, PMA stain); ¹H NMR (400 MHz, CDCl₃) δ 5.29 (d, 1H, J=1.6 Hz), 4.18-4.12 (m, 1H), 2.51 (t, 1H, J=8.8 Hz), 2.25-0.77 (m, 20H), 2.11 (s, 3H), 1.04 (s, 3H), 0.62 (s, 3H); ¹³C NMR (100 MHz, CDCl₃) δ 209.9, 147.4, 123.8, 68.1, 63.9, 56.5, 54.5, 44.3, 39.1, 37.5, 36.1, 35.6, 33.1, 32.3, 31.7, 29.6, 24.6, 22.9, 21.2, 19.1, 13.6.

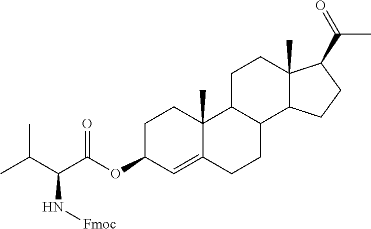

N-Fmoc-L-valine-3-β-progesterone (Fmoc 3a). An oven dried 50 mL RBF was charged with 90% 3-beta-hydroxy-progesterone (0.352 g, 1.00 mmol), N-Fmoc-L-valine (0.339 g, 1.00 mmol, 1.00 eq), and dimethylaminopyridine (DMAP) (0.0244 g, 0.200 mmol, 0.200 eq). The flask was sealed, evacuated, and inert gas flushed and 15 mL anhydrous dichloromethane was added, followed by addition of 1.10 mL (1.10 mmol, 1.10 eq) 1 M dicyclohexylcarbodiimide (DCC) in dichloromethane. The solution was stirred overnight then filtered through Celite. The filtrate was concentrated, prepared as a silica cake and eluted on a 40 g silica column with a 0-25% ethyl acetate in hexanes gradient. The main product was isolated as 0.554 g (87%) clear oil that foamed on drying. R_{f}=0.40 (1:1 EA/hex, PMA stain); ¹H NMR (600 MHz, CDCl₃) δ 7.78 (d, 2H, J=7.2 Hz), 7.63-7.61 (m, 2H), 7.41 (t, 2H, J=7.2 Hz), 7.33 (t, 2H, J=7.2 Hz), 5.35 (d, 1H, J=9.0 Hz), 5.31 (t, 1H, J=7.8 Hz), 5.21 (s, 1H), 4.40 (d, 2H, J=7.2 Hz), 4.31 (dd, 1H, J=9.0, 4.2 Hz), 4.25 (t, 1H, J=7.2 Hz), 2.52 (t, 1H, J=9.0 Hz), 2.23-2.16 (m, 3H), 2.12 (s, 3H), 2.05-1.96 (m, 3H), 1.78-1.55 (m, 6H), 1.50-1.33 (m, 4H), 1.25-1.10 (m, 2H), 1.06 (s, 3H), 1.00 (d, 3H, J=7.2 Hz), 0.93 (d, 3H, J=7.2 Hz), 0.90-0.79 (m, 2H), 0.64 (s, 3H).

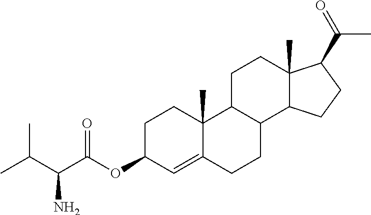

3-β-L-Valine-progesterone (3a). A 25 mL RBF was charged with 0.340 g (0.533 mmol) compound 3a. The flask was evacuated and inert gas flushed and 5 mL each of acetonitrile and dimethylformamide were added. A 0.527 mL (5.33 mmol, 10.0 eq) volume of piperidine was added and the clear colorless solution was stirred at room temperature for 30 min. The solvent was removed with addition of toluene for complete removal of DMF. A white solid formed that was redissolved in a minimum amount of toluene and loaded neat onto a 12 g silica column and eluted with 0-75% ea in hexanes. Main product containing fractions were combined and dried to give 0.196 g (89%) white foam. ¹H NMR (400 MHz, CDCl₃) δ 5.29-5.23 (m, 1H), 5.20 (d, 1H, J=1.6 Hz), 3.27 (d, 1H, J=4.8 Hz), 2.52 (t, 1H, J=9.2 Hz), 2.36-1.93 (m, 6H), 2.11 (s, 3H), 1.79-1.08 (m, 14H), 1.06 (s, 3H), 0.98 (d, 3H, J=6.8 Hz), 0.95-0.77 (m, 3H), 0.90 (d, 3H, J=6.8 Hz), 0.62 (s, 2H); ¹³C NMR (100 MHz, CDCl₃) δ 209.8, 175.6, 149.3, 119.3, 71.3, 63.8, 60.2, 56.4, 54.2, 44.3, 39.0, 37.5, 36.0, 35.2, 33.0, 32.3 (2 C), 31.7, 25.3, 24.6, 22.9, 21.1, 19.6, 19.0, 17.3, 13.6; IR (solid): 2934, 2843, 1724, 1705, 1384, 1354, 1166, 1146, 978, 873, 852 cm⁻¹; HRMS-ESI m/z 416.3156 ([M+H]⁺, C₂₆H₄₂NO₃ requires 416.3159).

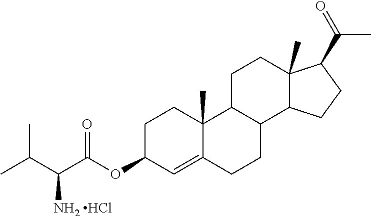

3β-L-Valine-progesterone-HCl (P1-31). A 10 mL RBF with stir bar was charged with 83 mg compound 4 and the flask was evacuated and flushed with argon. Anhydrous ether (2 mL) was added and the solution was chilled in an ice bath. Hydrogen chloride solution (0.10 mL 2.0 M in ether, 0.20 mmol, 1.0 eq) was added dropwise. A white precipitate formed in solution. The precipitate was filtered and washed with chilled ether. The product was recovered as 68 mg (75%) off-white solid.

### Example 1b

### C-20 Progesterone Derivatives

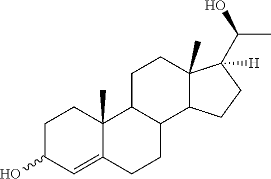

3,20-Hydroxy-progesterone (4a). An oven dried RBF was charged with 25 mL anhydrous THF and chilled in an ice bath. A 4.50 mL volume (9.00 mmol, 2.25 eq) of 2.0 M lithium aluminum hydride in THF was added. A separate ˜10 mL solution of progesterone (1.26 g, 4.00 mmol) in anhydrous THF was prepared in a dry flask. The solution was transferred to the reaction flask dropwise over 30 minutes. The mixture was heated under reflux for 1 h, cooled to room temperature, and quenched by the addition of ethyl acetate, followed by aqueous sodium sulfate. Solid sodium sulfate was added to remove excess water. The remaining salts were filtered and washed with THF. The organic filtrates were combined and concentrated to give 1.24 g (97%, recovered with 8% progesterone) white crystalline solid.

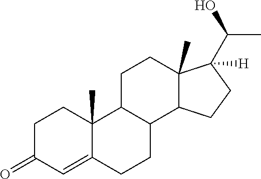

20-S-Hydroxy-progesterone (4). A 100 mL RBF was charged with 1.00 g crude compound 5 and 5.00 g manganese dioxide (activated by heating in oven for 2 days then cooled in a dessicator) and the reactants were suspended in 30 mL chloroform. The mixture was stirred at room temperature overnight. The mixture was then filtered through a pad of Celite and rinsed with chloroform. The clear, colorless filtrate was evaporated to dryness to give an off-white solid. The solid was recrystallized from ethyl acetate/hexane to give 0.565 g (57%) white solid.

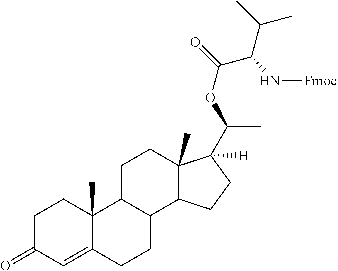

20-S—N-Fmoc-L-valine-progesterone (Fmoc 5a). An oven dried 50 mL RBF was charged with compound 4 (0.250 g, 0.790 mmol), N-Fmoc-L-valine (0.271 g, 0.798 mmol, 1.01 eq), and DMAP (0.010 g, 0.079 mmol, 0.100 eq). The flask was sealed, evacuated, and inert gas flushed and 10 mL anhydrous dichloromethane was added, followed by addition of 0.869 mL (0.434 mmol, 1.10 eq) 1 M DCC in dichloromethane. The solution was stirred overnight and then filtered through Celite and washed with dichloromethane. The crude product was loaded as a silica cake on a 40 g silica column and eluted with a 0-25% ethyl acetate in hexanes gradient over 45 min. Main product containing fractions were combined and dried under vacuum to give 0.436 g (87%) white foam.

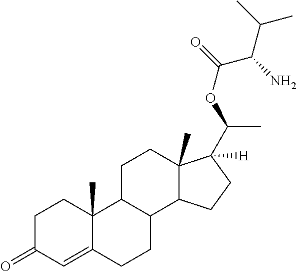

20-S-L-Valine-progesterone (5b). Compound 5a (0.374 g, 0.586 mmol) was dissolved in 6 mL anhydrous acetonitrile in a 25 mL RBF under argon. Piperidine (0.646 mL, 6.54 mmol, 10.0 eq) was added quickly dropwise at room temperature. A white clumping precipitate was observed in solution after 20 minutes. The precipitate was filtered and rinsed with acetonitrile. The filtrate was concentrated and the resulting white solid was redissolved in dichloromethane and concentrated in the presence of 1 g silica. The silica cake was eluted with 0-75% ethyl acetate in hexanes over 45 minutes on a 12 g silica column. Main product containing fractions were combined and concentrated to giveₛ a white solid. The solid was recrystallized from hexanes/ethyl acetate to give 0.097 g (40%) white powdery solid. R_{f}=0.06 (1:1 EA/hex); ¹H NMR (400 MHz, CDCl₃) δ 5.72 (s, 1H), 4.93-4.86 (m, 1 H), 3.23 (d, 1H, J=4.4 Hz), 2.46-2.23 (m, 5H), 2.10-0.80 (m, 18H), 1.17 (s, 3H), 1.16 (d, 3H, J=6.4 Hz), 0.98 (d, 3H, J=7.2 Hz), 0.88 (d, 3H, J=6.8 Hz), 0.68 (s, 3H); ¹³C NMR (100 MHz, CDCl₃) δ 199.8, 175.1, 171.5, 124.0, 73.4, 59.9, 55.4, 55.1, 54.0, 42.5, 39.2, 38.8, 35.9, 35.6, 34.2, 33.0, 32.2 (2 C), 25.6, 24.4, 21.1, 20.0, 19.5, 17.6, 17.1, 12.7; IR (film): 2933, 1721, 1672, 1381, 1187, 1071, 864 cm⁻¹; HRMS-ESI m/z 416.3156 ([M+H]⁺, C₂₆H₄₂NO₃ requires 416.3159).

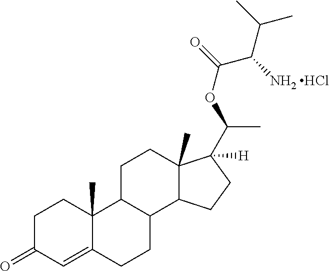

20-S-L-Valine-progesterone HCl salt (P1-57). Compound 8 (62 mg, 0.150 mmol) was dissolved in 1.5 mL anhydrous ether in a 5 mL RBF under argon and the solution was chilled in an ice bath. A 0.158 mL volume (0.158 mmol, 1.05 eq) of 1.00 M hydrochloric acid in diethyl ether was added. A precipitate formed in solution. The precipitate was filtered and washed with chilled ether to give 40 mg (59%) off-white solid.

### Example 1e

### Progesterone PD Series Compounds

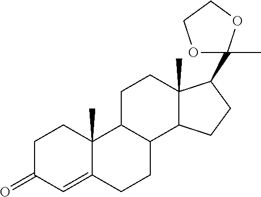

20-Ketal-progesterone (6). Progesterone (25.0 g, 79.5 mmol), oxalic acid (7.16 g, 79.5 mmol, 1.00 eq) and 350 mL benzene were added to a 1 L RBF with stir bar, followed by 75.4 mL (1.35 mol, 17.0 eq) ethylene glycol. The flask was fitted with a condenser topped Dean Stark apparatus and refluxed for 48 h. The solution was cooled and quenched with saturated sodium bicarbonate solution. The aqueous phase was extracted with benzene. The organic layers were combined and washed with DI. The organic layer was treated with magnesium sulfate to the point of free flowing solid and stirred at room temperature overnight. The solution was filtered and concentrated to give a sticky white solid. The solid was recrystallized from petroleum ether/acetone to give 9.30 g (31% at 94% purity) white solid.

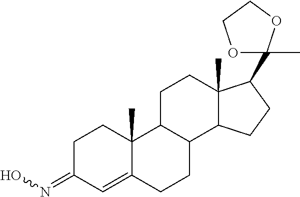

3-Hydroxy-oxime-20-ketal-progesteronc (7a/7b). Hydroxylamine HCl (2.78 g, 40.0 mmol, 4.00 eq) was added to a 100 mL oven dried RBF with 15 mL anhydrous dichloromethane. Triethylaminc (6.97 mL, 50.0 mmol, 5.00 eq) was added and the mixture was stirred for 45 minutes. Compound 6 was dissolved in 20 mL anhydrous DCM and added quickly dropwise to the reaction mixture. The reaction was stirred for 24 h at room temp. The solution was quenched with the addition of DI. The organic layer was washed with water. The aqueous washes were combined and extracted with dichloromethane. The organic layers were combined, dried, filtered, and concentrated with 10 g silica. The silica cake was eluted with a 0-25% ea in hex gradient over 60 minutes on a 120 g silica column. Main product were recovered as 2.23 g (60%) E oxime and 1.33 g (36%) Z oxime, both as white solids.

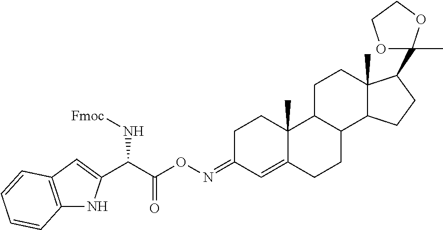

O—N-Fmoc-L-tryptophan-C3-oxime-C20-kctal-progesterone (8a). An oven dried 25 mL RBF was charged with oxime 11 (0.187 g, 0.500 mmol), N-Fmoc-L-tryptophan (0.242 g, 0.22 mmol, 1.05 eq), and DMAP (0.0061 g, 0.021 mmol, 0.10 eq). The flask was sealed, evacuated, and inert gas flushed and 15 mL anhydrous dichloromethane was added, followed after complete dissolution by addition of 0.550 mL (0.23 mmol, 1.10 eq) 1 M DCC in dichloromethane. The solution was stirred for 16 h at room temperature. The mixture was filtered through Celite, the filtrates concentrated, and the crude oil loaded as a silica cake onto 1.17 g silica. The cake was eluted on a 40 g silica column in 0-35% ea in hex over 90 minutes. The main product peak was isolated as 0.383 g (98%) white foam.

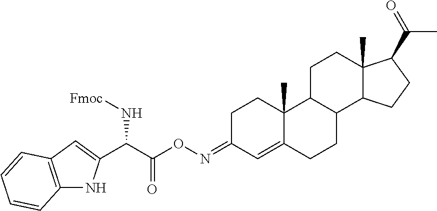

O—N-Fmoc-L-tryptophan-C3-oxime-progesterone (Fmoc 9a). Compound 8a (0.350 g, 0.448 mmol) was dissolved in 15 mL acetone and 0.0193 g (0.112 mmol, 0.250 eq) PTSA was added. The reaction was stirred at room temperature for 2.5 h. Ethyl acetate was added and the solvent was concentrated twice with re-addition of ethyl acetate. The ethyl acetate was washed with water (2×25 mL). The aqueous layers were combined and extracted with ethyl acetate. The organic layers were combined, washed with brine, dried, and concentrated to give a yellow oil that solidified on further drying. The mixture was redissolved in a minimum amount of DCM with toluene and loaded neat onto a 40 g silica column and eluted in a 0-40% ea in hex gradient over 70 minutes to give 0.315 g (95%) pale amber foam.

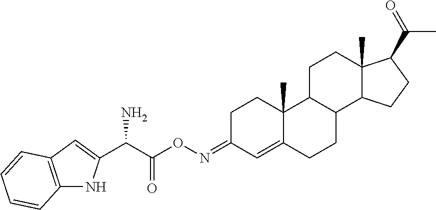

O-L-Tryptophan-C3-oxime-progesterone (9a). Compound Fmoc 9a (0.280 g, 0.379 mmol) was added to an oven dried 25 mL RBF. Anhydrous acetonitrile (7.5 mL) was added, followed by piperidine (0.141 mL, 1.42 mmol, 10.0 eq). The reaction was stirred for 30 minutes at room temperature. The solvent was removed by evaporation and the crude oil redissolved in toluene and reduced to dryness twice in succession. The crude off-white solid was redissolved in a minimum amount of DCM, loaded neat onto a 12 g silica column, and eluted with a 0-95% ea in hex gradient over 60 min. The main product was obtained as 0.120 g (61%) off-white solid.

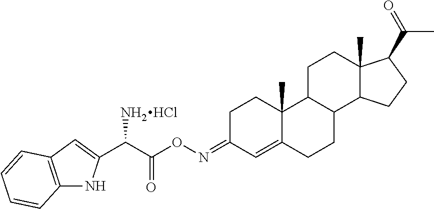

O-L-Tryptophan-C3-oxime-progesterone HCl salt (P1-79). Compound 14 (46 mg, 0.089 mmol) was dissolved in 2.5 mL anhydrous ether in a 10 mL RBF under argon. The solution was chilled in an icc bath and 0.195 mL 1 M HCl solution in ether was added. A white precipitate was observed to have immediately formed in solution. The mixture was filtered and the precipitate was washed with cold ether to give 21 mg (43%) white solid.

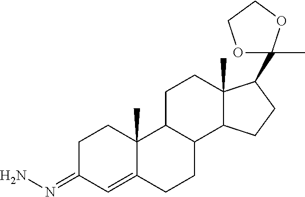

3-Hydrazine-20-ketal-progesterone (10). Compound 6 (0.377 g, 95% w/w, 1.00 mmol) was added to an oven dried 25 mL RBF and 5 mL absolute ethanol was added. Hydrazine (5.00 mL 1.0 M solution in THF, 5.00 mmol, 5.00 eq) was added which served to completely dissolve the starting material. This was stirred at room temperature for 1.5 h and set to reflux overnight. The solution was concentrated and dried under vacuum to give a white foam. Dichloromethane was added and the solution was re-concentrated to generate a solid that was filtered and washed with 3:1 hex/ether to give 0.164 g (44%) pale yellow crystals.

### Example 1d

### Allopregnanolone Derivatives

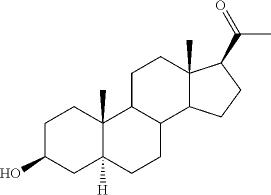

3β-hydroxy-5α-pregnan-20-one (12). An oven dried 500 mL RBF was charged with 10% palladium on carbon (0.400 g) and 5-pregnen-3-beta-ol-20-one (4.00 g, 12.6 mmol) and the flask was evacuated and flushed with argon. A 200 mL volume of absolute ethanol was added and the flask was flushed with hydrogen. The reaction was stirred at room temperature for 4 h. The mixture was filtered through Celite and the recovered clear, colorless filtrate was concentrated to reveal a white solid of mass 4.08 g. The solid was recrystallized from hexane/ethyl acetate (˜3:1 total 175 mL) to give 3.19 g white solid. A second recrystallization provided an additional 0.43 g for a total of 3.62 g (90%) white crystalline solid.

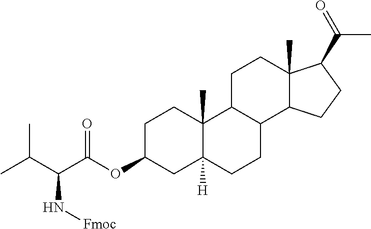

3β-N-Fmoc-L-valine-5α-pregnan-20-one (Fmoc 13a). An oven dried 25 mL RBF was charged with compound 20 (0.318 g, 1.00 mmol), N-Fmoc-L-valine (0.356 g, 1.05 mmol, 1.05 eq) and DMAP (12 mg, 0.100 mmol, 0.10 eq). The flask was sealed, evacuated and inert gas flushed, and 9 mL anhydrous DCM was added, followed after complete substrate dissolution by 1.10 mL (1.10 mmol, 1.10 eq) 1.0 M DCC in DCM. The reaction mixture was stirred at room temperature for 24 h. The mixture was filtered through Celite and rinsed with DCM. The sample was prepared as a silica cake and eluted on a 40 g silica column with 0-25% ea in hex over 45 min. Main product containing fractions were combined and isolated as 0.578 g (90%) white foam.

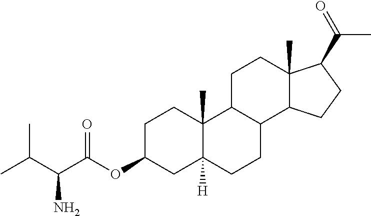

3β-L-vallne-5α-pregnan-20-one (13a). A 25 mL RBF was charged with compound Fmoc 13a (0.488 g, 0.725 mmol) and 7.5 mL acetonitrile. Piperidine (0.716 mL, 7.25 mmol, 10.0 eq) was added and the solution was stirred at room temperature for 30 min. Toluene was added and the solution was concentrated 3 times with addition of toluene. The resulting white solid was redissolved in a minimum amount of toluene and loaded onto a 12 g silica column. The column was eluted with 0-100% ea in hex over 40 minutes. The main product was obtained as 0.317 g (99%) clear/white semi-solid.

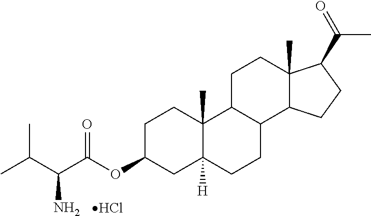

3β-L-valine-5α-pregnan-20-one HCl salt (P1-123). Compound 22 (0.317 g, 0.759 mmol) was dissolved in ˜2:1 anhydrous ether/DCM (6 mL total) under argon. The clear, slightly amber solution was chilled in an ice bath and 0.759 mL (0.759 mmol, 1.0 eq) 1 M HCl in ether solution was added slowly dropwise. A white precipitate was observed in solution. The solution was stirred at 0° C. for 30 min and then filtered. The precipitate was washed with ice chilled ether. The product was recovered as a slightly off-white solid of mass 0.175 g (51%).

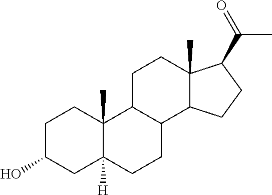

3α-hydroxy-5α-pregnan-20-one (14). An oven dried 100 mL RBF with magnetic stir bar was charged with 1.59 g (5.00 mmol) compound 20 and 15 mL anhydrous THF. Diethylazodicarboxylate (2.85 mL 40% soln. in toluene, 6.25 mmol, 1.25 eq) was added, followed by trifluoroacetic acid (0.482 mL, 6.25 mmol, 1.25 eq) and the flask was set in a room temperature water bath. To this pale amber suspension was added triphenylphosphine (1.64 g, 6.25 mmol, 1.25 eq). Sodium benzoate (0.901 g, 6.25 mmol, 1.25 eq) was then added and the suspension was stirred under argon for 24 h at room temperature. The THF was completely removed with methanol addition/cvaporation. Methanol (20 mL) was then added. The flask was fitted with a drying tube topped condenser and set for reflux. After 24 h, the methanol was removed and the remaining solid was redissolved in DCM. The organic layer was washed with DI (3×20 mL). The aqueOus layers were combined and extracted with DCM. The organic layers were combined, dried, filtered, and concentrated to give a white solid. The solid was prepared as a silica cake and eluted with 0-35% ea in hex on a 120 g silica column over 40 min. Main product containing fractions were combined and concentrated to give 1.46 g (92%) white solid.

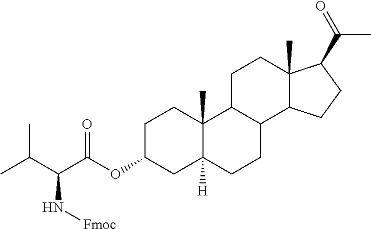

3α-N-Fmoc-L-valine-5α-pregnan-20-one (Fmoc 15a). An oven dried 50 mL RBF was charged with compound 14 (0.478 g, 1.50 mmol), N-Fmoc-L-valine (0.535 g, 1.58 mmol, 1.05 eq), and DMAP (18 mg, 0.150 mmol, 0.10 eq). The flask was sealed, evacuated and inert gas flushed, and 15 mL anhydrous DCM was added, followed after complete substrate dissolution by 1.65 mL (1.65 mmol, 1.10 eq) 1.0 M DCC in DCM. The flask was stirred at room temperature for 24 h. The mixture was filtered through Celite and rinsed with DCM. Silica (˜3 g) was added and the mixture was concentrated. The silica cake was eluted on a 40 g silica column with 0-25% ea in hex over 45 min. The main product was isolated as 0.834 g (87%) white foam.

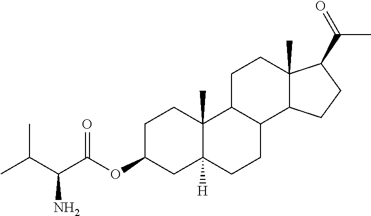

3α-L-Valine-5α-pregnan-20-one (15a). A 25 mL RBF was charged with compound Fmoc 15a (0.320 g, 0.500 mmol), 5 mL ACN, and 3 mL DMF. Piperidine (0.494 mL, 5.00 mmol, 10.0 eq) was added. The solution was stirred at room temperature for 30 minutes. Toluene was added and the solution was concentrated 3 times with addition of toluene. The pale amber oil was loaded in a minimum amount of toluene onto a 12 g silica column. The column was eluted with 0-100% ea in hex over 40 minutes. Main product fractions were combined to give 0.196 g (94%) sticky white solid.

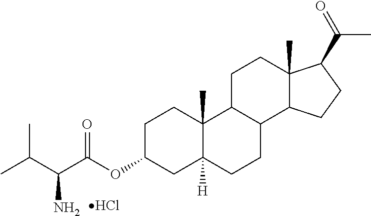

3α-L-Valine-5α-pregnan-20-one HCl salt (P1-131). Compound 25 (0.251 g, 0.600 mmol) was dissolved in 6 mL anhydrous ether under argon. The clear solution was chilled in an ice bath and 0.300 mL (0.600 mmol, 1.0 eq) 2.0 M HCl/ether solution was added slowly dropwise. A white precipitate was observed in solution. The solution was stirred at 0° C. for 30 min and then filtered. The precipitate was washed with ice chilled ether. The product was recovered as 0.150 g (55%) slightly off-white solid.

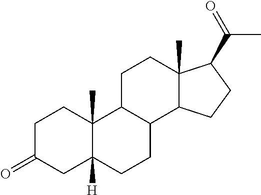

5β-Pregnane-3,20-dione (16). A three necked 500 mL RBF was charged with progesterone (2.00 g, 6.36 mmol), 5% Pd/CaCO₃ (0.180 g, 9% w/w), 200 mL absolute ethanol, and KOH (0.360 g in 1 mL DI). The flask was evacuated and flushed with hydrogen and the reaction stirred for 1 h. The ethanol was removed and the residue was redissolved in ether and washed with water. The water layer was extracted with ether (2×50 mL). The aqueous layer was then acidified to pH<3 with 1 M HCl and extracted with ether. The organic layers were combined, dried, filtered, and concentrated to give an off-white solid of mass 2.08 g. The sample was loaded in a minimum amount of toluene onto a 120 g silica column and eluted with 0-35% ea in hex gradient. The main product was recovered as 1.20 g (60%) white solid.

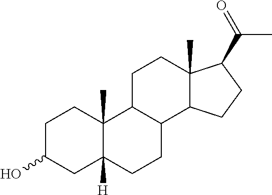

3-Hydroxy-5β-pregnane-20-one (17a/17b). A 250 mL RBF was charged with compound 26 (1.00 g, 3.16 mmol) and 40 mL absolute ethanol. The solution was warmed in an oil bath to 50° C. and sodium borohydride (0.179 g, 4.74 mmol, 1.50 eq) was added. The reaction was stirred for 10 min and 75-100 mL hot water was added until a slight cloudiness remained in solution. The solution was then allowed to cool gradually to room temperature and chilled in a 4° C. freezer for 3 h. The mixture was filtered and the white solid was washed with 30% ethanol in DL After drying, the recovered solids were loaded in a minimum amount of DCM onto a 120 g silica column and eluted with 0-25% ea/hex over 60 min. Main product containing fractions were combined and concentrated to give 0.710 g (71%) 3α-hydroxy-5β-pregnane-20-one and 0.110 g (11%) 3β-hydroxy-5β-pregnane-20-one isomer.

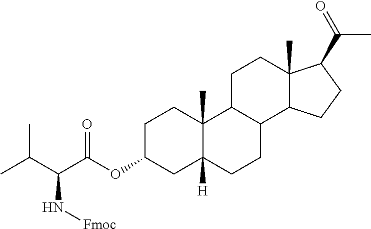

3α-N-Fmoc-L-valine-5β-1-pregnane-20-one (Fmoc 18a). An oven dried 25 mL RBF was charged with compound 27 (0.333 g, 1.05 mmol), N-Fmoc-L-valine-OH (0.373 g, 1.10 mmol, 1.05 eq), and DMAP (12.8 mg, 0.10 mmol, 0.10 eq). The flask was sealed, evacuated and inert gas flushed and 9 mL anhydrous DCM was added, followed after complete substrate dissolution by 1.15 mL (1.15 mmol, 1.10 eq) 1.0 M DCC in DCM. A white precipitate appeared in solution during DCC addition. The flask was stirred at room temperature for 24 h. The mixture was filtered through Celite and rinsed with DCM. The filtrate was concentrated with 2 g silica and the silica cake was eluted on a 40 g silica column with 0-25% ea in hex over 45 min. The main product was recovered as 0.541 g (81%) white foam.

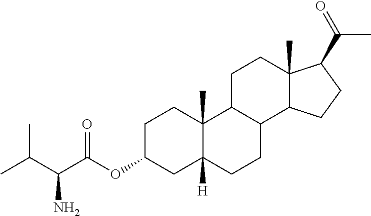

3α-L-valine-5β-pregnane-20-one (18a). A 25 mL RBF was charged with compound Fmoc 18a (0.500 g, 0.742 mmol) and 7 mL ACN. Piperidine (0.733 mL, 7.42 mmol, 10.0 eq) was added and the solution was stirred at room temperature for 30 minutes. A flaky white precipitate appeared in solution. The precipitate was filtered and washed with ACN. The organic layers were combined with toluene and the solution was concentrated 3 times with addition of toluene. The white solid was redissolved in a minimum amount of toluene and loaded onto a 12 g silica column. The column was eluted with 0-75% ea in hex over 40 minutes. The main product was isolated as 0.301 g (97%) white solid.

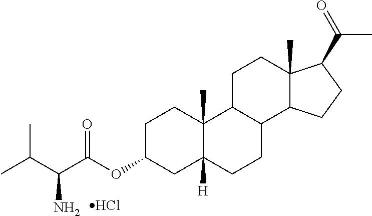

3α-L-Valine-5β-pregnane-20-one HCl salt (P1-133). Compound 30 (0.155 g, 0.371 mmol) was dissolved in 4 mL anhydrous ether under argon. The clear solution was chilled in an ice bath and 0.186 mL (0.371 mmol, 1.0 eq) 2 M HCl in ether solution was added slowly dropwise. A white precipitate was observed in solution. The solution was stirred at 0° C. for 30 minutes and then filtered. The precipitate was washed with ice chilled 2:1 hex/ether. The product was recovered as a slightly off-white solid of mass 0.120 g (71%).

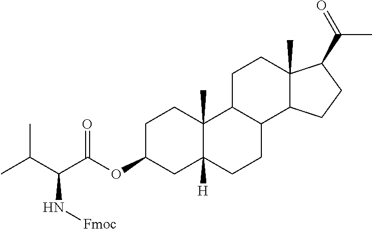

3β-N-Fmoc-L-valine-5β-pregnane-20-one (Fmoc 18b). An oven dried 25 mL RBF was charged with compound 28 (0.234 g, 0.735 mmol), N-Fmoc-L-valine (0.262 g, 1.10 mmol, 1.05 eq) and DMAP (9 mg, 0.10 mmol, 0.10 eq). The flask was sealed, evacuated and inert gas flushed, and 8 mL anhydrous DCM was added, followed after complete substrate dissolution by 0.808 mL (0.808 mmol, 1.10 eq) 1.0 M DCC in DCM. The flask was stirred at room temperature for 24 h. The mixture was concentrated with 1.5 g silica, and the silica cake was eluted on a 40 g silica column with 0-25% ea in hex over 45 min. The main product was isolated as 0.345 g (73%) white foam.

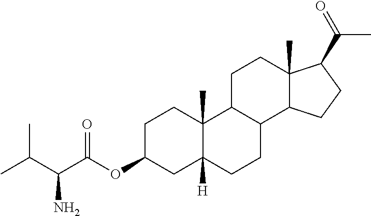

3β-L-Valine-5β-pregnane-20-one (18b). A 25 mL RBF was charged with compound 31 (0.307 g, 0.456 mmol) and dissolved in 7 mL ACN. Piperidine (0.450 mL, 4.56 mmol, 10.0 eq) was added and the solution was stirred at room temperature for 15 minutes. The precipitate was filtered and washed with ACN. The organic layers were combined with toluene and the solution was concentrated 3 times. The white solid was redissolved in a minimum amount of toluene, loaded onto a 12 g silica column, and eluted with 0-75% ea in hex over 35 minutes. The product was obtained as 0.176 g (93%) white foam.

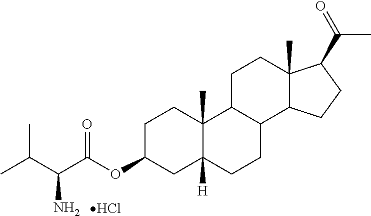

3β-L-Valine-5β-pregnane-20-one HCl salt (P1-135). Compound 32 (0.123 g, 0.290 mmol) was dissolved in 3 mL anhydrous ether under argon. The clear solution was chilled in an ice bath and 0.15 mL (0.29 mmol, 1.0 eq) 2 M HCl in ether solution was added slowly dropwise. A white precipitate was observed in solution. The solution was stirred at 0° C. for 30 minutes and then filtered. The precipitate was washed with ice chilled 2:1 hex/ether. The product was recovered as a slightly off-white solid of mass 0.052 g (39%).

### Example 2

### Effectiveness of Certain Steroid Analogues in Reducing Post-Injury Edema

The methodology used is described in VanLandingham et al., Neuropharmacology, 2006, 51, 1078-1085.

### Surgery:

Sterile surgical procedures were used to prevent animal infection. Rats (20-month old Fischer 344 rats, which are the “human” equivalent of about 60 years old) were anesthetized and maintained on isoflurane and an equivalent amount of NO₂ and O₂ for 3 min prior to surgery. After brain contusion, O₂ levels were doubled compared to NO₂ and maintained through the reainder of the surgery procedure. A stereotaxic apparatus was used to stabilize the head in a horizontal position. Core body temperature was monitored and maintained at 37° C. using a Harvard homeothermic blanket (Harvard Apparatus, Holliston, Mass.). There was no direct measure used to deterct brain temperature. Blood oxygen and heart rate were maintained using a SurgiVet monitor (SurgiVet, Waukesha, Wis.) and maintained above 90% and 340 bpm respectively. A midline incision was made and the scalp retracted. A bilateral 6-mm craniotomy was performed with surgical drill centered at 3 mm rostral to bregma. The stainless steel impactor was positioned over the MFC at 3.0 mm A/P and 0.0 M/L. These coordinates represent the MFC as described by Paxinos and Watson (The Rat Brain in Stereotaxic Coordinates, Academic Press, San Diego, 1986). The cortical injury was induced using a pneumatically controlled device (Hoffman et al., J. Neurotrauma, 1994, 11, 417-431). Brain impact duration was 0.5 s. using a 5-mm impactor tip with a velocity set at 2.125 m/s and a cortical depth of 2 mm. Following the contusion, bleeding was halded and fascia and scalp were sutured shut. After surgery, animals were allowed to recover from anesthesia on a homeothermic heating blanket in a holding cage until awake. sham surgeries controlled for anesthesia and stress. All surgical procedures were the same, except that sham rates were not given a craniotomy or cortical injury. Previous studies using craniotomy as a control found no differences between shams with or without this procedure. (Goss et al., Pharmacol. Biochem. Behay. 2003, 76(2), 231-242).

All experimental treatments given by injection (progesterone and progesterone analogues # 31, #57 and #79) were made in stock solutions using 2-Hydroxypropyl-b-cyclodextrin (HBC; 45% w/v solution in H₂O) as the solvent. These experimental solutions were then diluted 1:1 with sterile water for a final concentration of HBC of 22.5%.

### Treatment Protocol:

All injections were done at the same time with brain harvesting at 24 h post-injury. Rats in each group were weightd prior to treatment to ensure proper dosage. The first injection at 1 h after surgery was given intraperitoneally to ensure rapid adsorption. All subsequent injections were made subcutaneously for gradual adsorption at 6 and 24 h. Injection times and neurosteroid doses were based on previous results of neurosteroid treatment (Roof et al., Twenty First Annual Meeting of the Society for Neuroscience, Miami Beach Fla., p. 191 and He et al., Exp. Neurol. 2004, 189, 404-412). The sterioids were dissolved in vehicle (22.5% 2-hydroxypropyl-b-cyclodextrin solution) at 4 mg/kg. The sham group received no treatment and injury control group received vehicle only.

### Cerebral Edema Analysis:

At 24 h after traumatic brain injury flresh brains were extracted from the skull and the dorsal cerebrum was separated along the line of the lateral fissure. Four 3-mm coronal sections were cut rostral to caudal, placed in pre-weighed 1.5 mL tubes and re-weighed (wet weight). Tubes were then left uncapped and placed in a vacuum oven set at 60° C. with an atmospheric pressure of 0.3 for 48 h. Following tube recapping, the tissue samples were again weighed (dry weight). Cerebral edema (% water content) was determined as the difference in wet and dry weights divided by wet weight (Roof and Stein Restor. Neurol. Ncurosci., 1992, 4, 425-427). Edema measures are reflective of the difference inwater content between at the average of the two most rostral (injury region) segments and most caudal (occipital cortex) segments of the dorsal sections of the brain.

### Results:

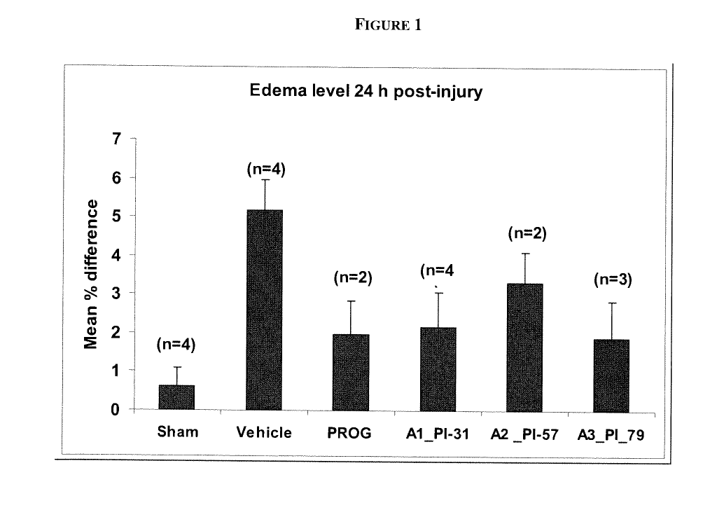

FIG. 1 shows the % difference edema results for brain tissue after 24 hours post brain injury. The mean % difference calculated for sham, vehicle, progesterone, Compound 31, Compound 57 and Compound 79 subjects were 0.6%, 1.2%, 2.0%, 2.2%, 3.3% and 1.9%, respectively. Samples treated with progesterone, and Compounds 31, 57 and 79 all showed a decrease in edema compared to subjects treated with vehicle.

### Example 3

### Effects of Vitamin D Deficiency on Efficacy of Progesterone Treatment—Materials and Methods

Eighty-seven 20-month-old male Fischer 344 rats (the “human” equivalent of about 60 years old) weighing 450-550 g at the time of injury were used in this experiment. Animals were housed and handled as previously described (Cutler et al., 2007). The animals in this study were separated into two groups, vitamin D normal (D-normal) and vitamin D deficient (D-deficient). The D-normal group was given standard rat chow used in our animal care facility (Rodent Diet 5001, LabDiet®, St. Louis, Mo.). The D-dcficient group was fed a vitamin D-null version of the same diet (Diet 5A4Y, modified 5001 with no D3, TestDiet®, Richmond, Ind.); all rats were weighed daily to ensure constant energy intake. Animals in the D-deficient group were maintained on the diet for at least 21 days prior to surgery. Eight days has been shown to be sufficient time to induce a circulating 25-hydroxyvitamin D3 level consistent with deficiency (Narayanan et al., 2004), but the time period was extended to allow the sequelae of the D-deficiency to become apparent and to provide a better model for the human population. For this same reason the null diet was not altered in any other way, and the rats assigned to the D-deficient group were maintained on it until they were killed for harvesting of brain tissue. Since vitamin D is activated by UVB light (280-315 nm wavelength), the overhead lights were modified to not produce radiation in this range.

### Surgery and Contusion Injury

Rats were anesthetized using isoflurane gas (5.0% induction, 1.0-1.5% maintenance, 700 mmHg N2O, 500 mmHg O2) and surgery was performed using aseptic techniques as previously described (Cutler et al., 2007). Briefly, a 6 mm diameter mid-sagittal bilateral craniotomy was performed 3 mm anterior to bregma and a cortical contusion injury (CCl) was produced in the medial frontal cortex (MFC) by a pneumatic cortical contusion device (5 mm diameter) with impact velocity of 2.25 m/s, impact time of 500 ms, and depth of 3.5 mm ventral to bregma. The incision was sutured closed after all bleeding had fully stopped. In the sham group, the incisions were sutured closed after comparable time under anesthesia. Animals dehydrated due to blood loss were given 3 mL of lactated Ringer's solution subcutaneously within 6 hours of injury.

### Treatment

Animals were assigned to D-normal or D-deficicnt groups. Normal animals were assigned to one of three groups (n=5/group): Sham (SHAM), Vehicle (VH), and Progesterone (PROG). Deficient animals were assigned to one of five groups (n=5/group): Sham (SHAM), Vehicle (VH), Progesterone (PROG), Progesterone with VDH (D+PROG), and VDH alone (D). The same assignment was followed for both 24-hour and 72-hour survival groups. The treatments were: VH: 22.5% 2-hydroxypropyl-β-cyclodextrin; PROG: 16 mg/kg PROG (P0130, Sigma-Aldrich, St. Louis, Mo.); D+PROG: 16 mg/kg PROG combined with 5 mg/kg VDH (D1530, Sigma-Aldrich) for the first injection and 16 mg/kg PROG with equivalent volume VII for the rest; D: 5 mg/kg VDH for the first injection and vehicle for the rest. A previously published treatment protocol was used (Cutler et al., 2007) consisting of an intraperitoneal injection 1 hour post-injury followed by subcutaneous injections at 6 hours, 24 hours, and every 24 hours thereafter until the animals were killed. All drug treatments were dissolved in vehicle, and injection volume was equally proportional to each animal's weight across all groups. The intact sham (SHAM) groups served to provide baseline data and therefore received no injury or injections. 16 mg/kg PROG was used because previous research demonstrated it to be the most effective dosage in young and aged rats (Cutler et al., 2007; Goss et al., 2003). Animals receiving VDH treatment were given only a single 5 mg/kg VDH injection 1 hour post-injury based on the evidence that a single megadose of VDH can reverse deficiency (Diamond et al., 2005).

### Tissue Preparation and Western Blot Analysis

Animals were killed 24 or 72 hours after surgery with a lethal dose of Nembutal (1 mL) and decapitated. Their brains were prepared for protein analysis and Western blots were performed as previously described (Cutler et al., 2007), using 15 μL of each sample (30 μg protein) per well in 18-well 4-20% Tris-HCL acrylamide Criterion Gels (BioRad, Hercules, Calif.). The primary antibodies used in this experiment were TNFα (AB1837P, Millipore/Chemicon, Temecula, Calif.), IL-1β (ab9787, Abcam Inc., Cambridge, Mass.), IL-6 (Abeam, ab6672), NFκB p65 (#3034, Cell Signaling Inc., Danvers, Mass.), COX-2 (Abeam, ab6665), p53 (Cell Signaling, #9282), cleaved caspase-3 (Asp175; Cell Signaling, #9661S), and β-actin (Abeam, ab37063).

### Statistical Analysis

All results were expressed as the mean+/− the standard error of the mean (SEM). Statistical significance was set a priori at p<0.05 and data were analyzed using ttests, Pearson correlations, one-way analysis of variance (ANOVA) with Tukey-HSD post hoc tests, and general linear models (GLMs). All analyses were calculated using SPSSTM 15.0 statistical analysis software.

### Example 4

### Vitamin D Deficiency Increases CNS Inflammatory Responses

The Vitamin D deficient animals were observed to be more “frail” in comparison with rats fed the normal diet. Although these observations were not always blinded, deficient animals generally bled longer (indicating a possible coagulation problem), displayed softer bones (i.e., the skull was easier to drill through), showed less stable vital signs during surgery, and required a lower concentration of isoflurane to become unconscious. They also took longer to recover after surgery and were observed to be less active when handled for treatment, injections and weighing.

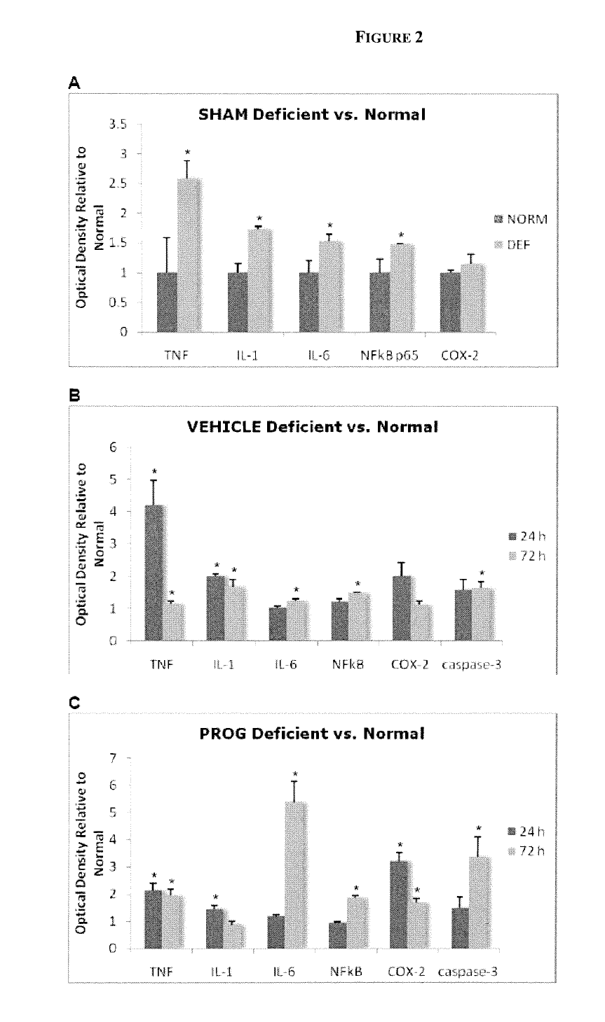

FIG. 2A shows the relative levels of inflammatory proteins (TNFα, IL-1β, IL-6, NFκB p65, COX-2) in the MFC of animals maintained on a D-deficient diet compared to animals fed a normal diet. All cytokines were normalized respectively to those found in normal (vertical axis value=1) and are shown as the ratio of deficient:normal±SEM. T-test p-values comparing deficient versus normal animals were: TNFα (p=0.026), IL-1β (p=0.002), IL-6 (p=0.047), NFκB p65 (p=0.036), COX-2 (p=0.26). With the exception of COX-2, all inflammatory cytokines measured were significantly elevated in the intact D-deficient rats compared to intact D-normal animals.

### Example 5

### Vitamin D Deficiency Exacerbates Injury in Animals with TBI

FIG. 2B shows the results for each of the inflammatory proteins identified above 24 and 72 hours after injury. The data were normalized to the respective cytokine at the same time-point in D-normal animals (vertical axis value=1) and are shown as the ratio deficient:normal±SEM. At 24 and 72 hours, respectively, the t-test p-values comparing normal and deficient animals were: TNFα (p=0.029; p=0.039), IL-1β (p=0.015; p=0.044), IL-6 (p=0.35; p=0.013), NFκB p65 (p=0.22; p<0.001), COX-2 (p=0.097; p=0.20), cleaved caspase-3 (p=0.035; p=0.009). At 24 hours after injury, only TNFα and IL-1β were significantly elevated in D-deficient animals treated with vehicle compared to their D-normal counterparts. By 72 hours, however, all inflammatory markers with the exception of COX-2 were significantly higher in vehicle-treated D-deficient versus D-normal animals.

A similar result was seen in animals treated with PROG. FIG. 2C shows the results for the same proteins in deficient versus normal PROG-treated animals 24 and 72 hours after TBI. The data are normalized to the respective cytokine at the same time point in normal animals (vertical axis value=1) and are shown as the ratio deficient:normal±SEM. At 24 and 72 hours the t-test p-values were: TNFα (p=0.006; p=0.006), IL-1β (p=0.016; p=0.30), IL-6 (p=0.11; p=0.001), NFκB p65 (p=0.17; p=0.003), COX-2 (p=0.001; p=0.02), cleaved caspase-3 (p=0.23; p=0.019). At 24 hours after injury, TNFα, IL-1β, and COX-2 are elevated in D-deficient versus D-normal animals treated with PROG, but by 72 hours all except IL-1β are higher in the deficient group. This may suggest that effects of D-deficiency become more pronounced as the injury evolves over time.

### Example 6

### Vitamin D Deficiency Attenuates the Beneficial Effects of PROG after TBI, but Cotreatment with VDH Improves Outcome in Deficient Animals

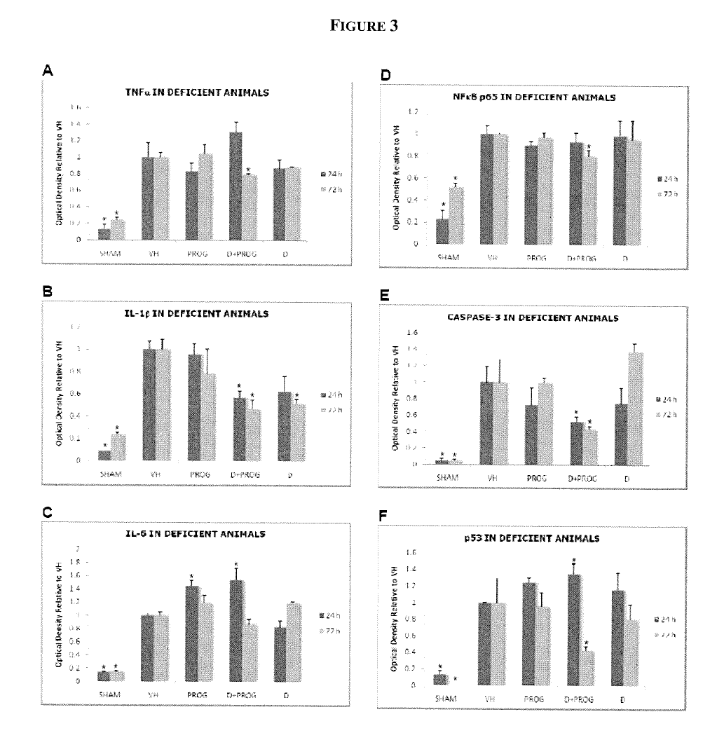

FIG. 3 shows that PROG treatment in D-deficient animals alone results in mild improvement compared to vehicle-treated D-dcficicnt animals, but its effects were minimal compared to the significant improvements seen when it is given with VDH. Panels A-D in FIG. 3 show the relative levels for several cytokines 24 h and 72 h after TBI in D-deficient animals. All values are normalized to the vehicle-treated group average for each timepoint: TNFα (FIG. 3A 24 h: F=8.530, p=0.001; 72 h: F=26.931, p<0.001), IL-1β (FIG. 3B, 24 h: F=11.781,p=0.001; 72 h: F=9.555, p=0.001), IL-6 (FIG. 3C, 24 h: F=16.481, p<0.001; 72 h: F=32.067, p<0.001), NFκB p65 (FIG. 3D, 24 h: F=9.960, p=0.001; 72 h: F=9.707, p<0.001). In most cases, only D+PROG treatment resulted in significant reduction of inflammation by 72 hours after injury, suggesting vitamin D may interact with both the injury process and PROG treatment.

### Example 7

### Administration of VDH with PROG in Vitamin D Deficient Animals Reduces Cell Death and DNA Damage Compared to Vehicle, VDH, or PROG Alone

The two molecular endpoints examined in this study were levels of activated caspase-3, the final effector in the apoptotic pathway, and p53, a cell-cycle control protein elevated by DNA damage and involved in the cellular choice between apoptotic cell death and DNA repair processes (Offer et al., 2002). Since vitamin D is known to increase p53 expression (Gupta et al., 2007), we measured the ratio of altered to normal p53 as an indicator of DNA damage (Offer et al., 2002). Our results (FIGS. 3E, F) show a significant decrease in activated caspase-3 (24 h, F=6.681, p=0.007; 72 h, F=10.756, p=0.001) and a bidirectional effect on p53-DNA interaction (24 h, F=6.563, p=0.003; 72 h, F=6.181, p<0.001) only in animals treated with D+PROG. These results suggest that the combined D+PROG treatment is the most effective in reducing cell death and DNA damage after TBI in D-deficient animals.

Interestingly, at 24 h, TNFα IL-6, and NFκB p65 were negatively correlated with p53 (p<0.05), suggesting that higher levels of these proteins may be beneficial in the very short term. This effect was reversed at 72 h, when these proteins were positively correlated with DNA damage (p<0.05) and cell death (p<0.01).

### Example 8

### Combined Treatment with PROG and VDH Improves Behavioral Function Compared to Treatment with Vehicle, PROG, or VDH Alone

In addition to molecular measures of inflammatory cytokines, the behavioral effects of the various treatments were examined. Since this study was limited to the short term effects on inflammation, only short-term Spontaneous Locomotor Activity was used.

Spontaneous locomotor activity was performed as previously described (Cutler et al., 2007). The spontaneous locomotor activity task has previously been shown to be sensitive to our model of TBI and to the effects of PROG treatment (Cutler et al., 2007), as well as to potential behavioral and motor derangements due to D-deficiency in open-field testing (Kalueff et al., 2004b).

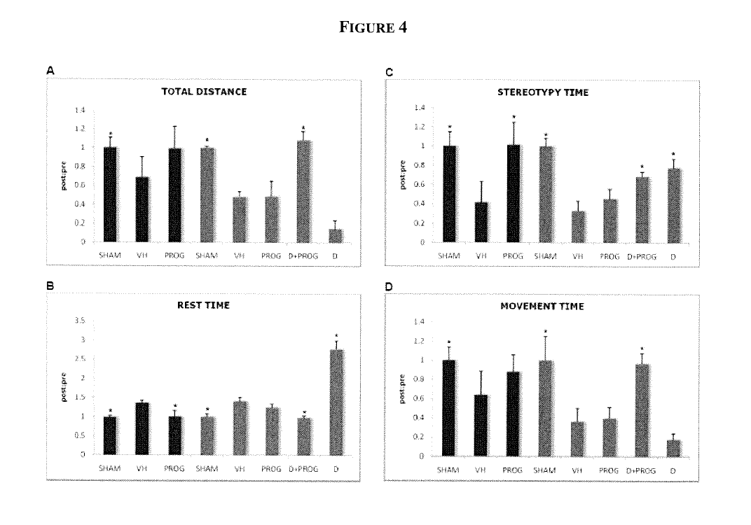

The results are shown by the panels in FIG. 4 as the ratios of post-injury:preinjury measurements and are normalized to sham animals to control for the variability in different animal squads. The basic parameters examined were total distance (FIG. 4A) (TOTDIST, F=3.356, p=0.014), resting time (FIG. 4B) (RESTIME, F=26.340, p<0.001), stereotypy time (FIG. 4C) (STRTIME, F=4.017, p=0.006), and movement time (FIG. 4D) (MOVTIME, F=2.806, p=0.028) 72 hours after injury. Significant improvement in locomotor activity with combination D+PROG treatment were observed, but little or no benefit with either PROG or VDH alone. Most behavioral parameters showed significant negative correlations with both cleaved caspase-3 and p53 (p<0.05). Regression analyses further showed that the various behavioral parameters were well accounted for (p<0.05) by models using deficiency/injury/treatment as fixed factors with normalized molecular measures as covariates, suggesting a relationship between the molecular acute inflammatory response and behavioral performance.

To summarize, (1) vitamin D deficiency increases baseline inflammation in the brains of uninjured aged rats, potentially establishing a detrimental underlying condition; (2) vitamin D deficiency increases a number of inflammatory markers after injury in aged rats treated with vehicle at both 24 and 72 hours; (3) in aged rats with brain injury, progresterone is effective in reducing acute inflammation, a key indicator of survival in human patients; (4) vitamin D deficiency increases acute phase inflammation and attenuates the benefits of progesterone treatment in aged rats with TBI, suggesting that such a deficiency could increase mortality after brain injury in human patients; (5) a combination of progesterone and vitamin D exhibited non-linear synergistic effect, and partially reverses the effects of vitamin D deficiency and reduces post-TBI acute inflammation in old rats; (6) in vitamin D deficient aged rats with TBI, the only treatment that reduced proteins measured (TNFα, IL-1β, IL-6, NFκB p65, activated caspase-3, p53) in all cases by 72 hours after injury was the combination of progesterone and vitamin d (5 mg/kg in a single dose) compared to vehicle or either compound given alone; (7) the combination treatment was also the only one that dramatically improved behavioral parameters, which statistical models (not shown) showed to be strongly correlated with systemic inflammation and levels of TNFα and IL-6.

Thus, vitamin D deficiency can significantly exacerbate acute CNS inflammation and attenuate the benefits of progesterone treatment after TBI. Progesterone regains its efficacy, however, when the deficiency is corrected by co-treatment with vitamin D. Thus, a combination treatment with progestereone and vitamin D given to patients (particularly the cicderly or others at risk of vitamin D deficiency) with TBI should improve survival over progesterone given alone to the same population.

### Example 9

### Dosing Evaluation with PROG and VDH on E18 Rat Primary Cortical Neurons

### A. Summary

In this study, E18 rat primary cortical neurons were pre-treated with different concentrations of progesterone (PROG) and 1,25-dihydroxyvitamin D3 hormone (VDH) separately or in combination for 24 hours and then exposed to glutamate (0.5 plvI) for the next 24 hours. Lactate dehydrogenase (LDH) release and 3-(4,5-dimethylthiazol-2-yl)-2,5-diphenyltetrazolium bromide (MTT) reduction assays were used to measure cell death.

Both PROG and VDH significantly (P<0.001) reduced neuronal loss when tested independently. Primary cortical cultures treated with VDH exhibited a “U-shaped” concentration-response curve. PROG at 20 μM and VDH at 100 nM concentration were most neuroprotective. When the drugs were combined, the “best” doses of PROG (20 μM) and VDH (100 nM), used individually, did not show substantial efficacy; rather, the lower dose of VDH (20 nM) was most effective when used in combination with PROG (P<0.01).

The effect of combinatorial treatment on MAPK activation as a potential neuroprotective mechanism was also studied. It was shown that PROG and VDH activated MAPK alone and in combination. The best combination dose of FROG and VDH (20 μM and 20 nM, respectively), as observed in cell death assays (LDH and Men), resulted in more increase in MAPK activation when compared with either the most individually neuroprotective concentration of PROG (20 μM) and VDH (100 nM) or the combination of these individual best doses.

### B. Materials and Methods

1. Neuronal Culture

NeuroPure™ E18 primary rat cortical cells were commercially procured (Catalogue # N200200, Genlantis, San Diego, Calif., USA) as micro-surgically dissected regions from day 18 embryonic Sprague-Dawley rat brain. The tissues were processed for culturing according to manufacturer specifications. Briefly, enzymatic pre-treatment of the tissue was done prior to mechanical dissociation by incubating the tissues in sterile NeuroPapain enzyme solution at 30° C. for 30 minutes. Following incubation, the cells were centrifuged and transferred to fresh plating medium, where they were then dissociated into isolated neurons using a P-1000 pipettor with a sterile 1 ml plastic tip (0.8-1.0 mm diameter opening). The cells were again centrifuged and seeded in multi-well plates pre-coated with poly-D-lysine (0.15 ml/cm², 50 μg/ml) and maintained at 37° C. in a humidified 5% CO2 atmosphere. All experiments were performed after 9-10 days in culture.

2. Induction of Glutamate Excitotoxicity and Drug Treatment

Twenty-four hours before glutamate exposure, cultures were pre-treated with both PROG (Cat. #P3972; Sigma Aldrich, St. Louis, Mo., USA) and VDH(Cat. #D1530; Sigma) separately or in combination with VDH at various concentrations. Stock solutions of PROG and VDH were prepared in dimethylsulfoxide (DMSO; Cat. #D2650; Sigma) and ethanol respectively, both of which were further diluted in culture medium so that the final concentrations of DMSO and VDH were <50 l/ml and 0.01% respectively. Glutamate was diluted in phosphate-buffered saline (PBS, pH 7.4). All reagents were filter sterilized before being added to cultures.

At Day 11, cortical neurons in fresh media were separated into five treatment groups: (i) control; (ii) 24 hour treatment with 0.5 μM glutamate (Sribnick E A, et al., (2004) J Neurosci Res 76: 688-696); (iii) 24 hour pre-treatment with different concentrations of PROG (1, 5, 10, 20, 40, 80 nM) with subsequent exposure to glutamate for 24 hours; (iv) 24 hour pre-treatment with VDH (1, 5, 10, 20, 40, 80, 100 nM) with subsequent exposure to glutamate for 24 hours; (v) 24 hour pre-treatment with different combinations of PROG and VDH (PROG: 20 μM+VDH: 1, 5, 10, 20, 40, 80, 100 nM) with subsequent exposure to glutamate for 24 hours.

3. Evaluation of Neuronal Death

Two widely accepted assays (LDH release and MTT reduction assay) for the measurement of cell viability were used. These assays are considered very reliable and reproducible with high predictive validity and are widely used in various pharmacological studies (Nilsen J, et al., (2002) Endocrinology 143: 205-212.).

The LDH assay was performed as follows. Cytotoxicity was assessed 24 hours after the start of the exposure by quantitative measurement of LDH in the bathing medium, an index that is proportional to the total number of neurons damaged by excitotoxic exposure (Koh J Y, et al., (1987) J Neurosci Methods 20: 83-90). LDH activity was measured using a Cytotoxicity Detection Kit (Roche Molecular Biochemicals, Indianapolis, Ind., USA) and quantitated by measuring absorbance at 490 nm. Data were normalized against the amount of LDH activity released from vehicle-treated control cultures receiving no glutamate.

Neuronal death was also assessed by MTT [3-(4,5-dimethylthiazol-2-yl)-2,5-diphenyltetrazolium bromide] assay, which is based on the cleavage of the tetrazolium ring of the pale yellow MTT into dark blue formazan crystals by mitochondrial dehydrogenase enzyme in viable cells. These blue formazan crystals accumulate within the cells due to their impermeability to cell membrane, and are then solubilized by adding DMSO. The intensity of blue colored formazan solution is directly proportional to the number of surviving cells. Concentrations were determined by photometric analysis. Briefly, 10 μl of MTT were added per well and incubated at 37° C. for 4 hours until purple precipitate was visible. DMSO (50 μl) was added to solubilize the crystals and the absorbance was read at 570 nm.

4. Morphological Analysis of Cortical Cultures

Changes in the morphology of neurons treated with different drugs in various groups were observed using a phase-contrast microscope (Nikon). Primary cultures were mainly observed for neurite outgrowth, a hallmark feature of healthy cells, and the density of healthy cells in different groups.

5. MAPK Phosphorylation

PROG and VDH were added to the primary cultures, as described above, for 30 minutes (Nilsen J, et al., (2002) Endocrinology 143: 205-212) and the cells were lysed using RIPA lysis buffer kit (sc-24948, Santa Cruz, Calif., USA). Protein was determined in cell lysates by bicinchoninic acid (BCA) protein assay (Cat. # 23225, Pierce, Rockford, Ill., USA). Cell lysates (40 μg protein each sample) were separated under reducing and denaturing conditions by 12.5% acrylamide Criterion gel (BioRad, Hercules, Calif., USA) at 200V for 1 hour and transferred to a polyvinylidene difluoride (PVDF) membrane at 100V for 30 minutes. The non-specific binding sites of the membrane were blocked with 5% non-fat dry milk in PBS-T (phosphate buffered saline containing 0.05% Tween-20). For MAPK phosphorylation, membrane was probed with p-ERK1/2 antibody (sc-101761, Santa Cruz) recognizing the dual threonine (Thr 202) and tyrosine (Tyr 204) phosphorylation sequence from MAPK. Total ERK1/2 protein was detected using ERK2 (C-14) antibody (sc-154, Santa Cruz). Membranes were then incubated in horseradish peroxidase (HRP)-conjugated secondary antibody (Goat anti-rabbit IgG; 074-1506, KPL, Gaithersburg, Md., USA). β-actin was probed as a loading control. Blots were developed using a chemiluminescent substrate (Pierce) for 5 minutes. Chemiluminescent bands were detected on a Kodak autoradiography film in a dark room and their densities were measured using Bio-Rad Gel-Doc software “Quantity-One 4.6.1.” MAPK activation was calculated by normalizing p-ERK1/2 with total ERK1/2 protein values.

6. Statistical Analysis of Data

Analysis of variance (ANOVA) and post-hoc tests were employed. The Neuman-Keuls test was used for independent comparisons among groups. The significance of results was set at P<0.05 two-tailed. All data are presented as mean±standard error of the mean (SEM).

### C. Results

1. Neuroprotective Effect of PROG Against Glutamate-Induced Excitotoxic Cell Death

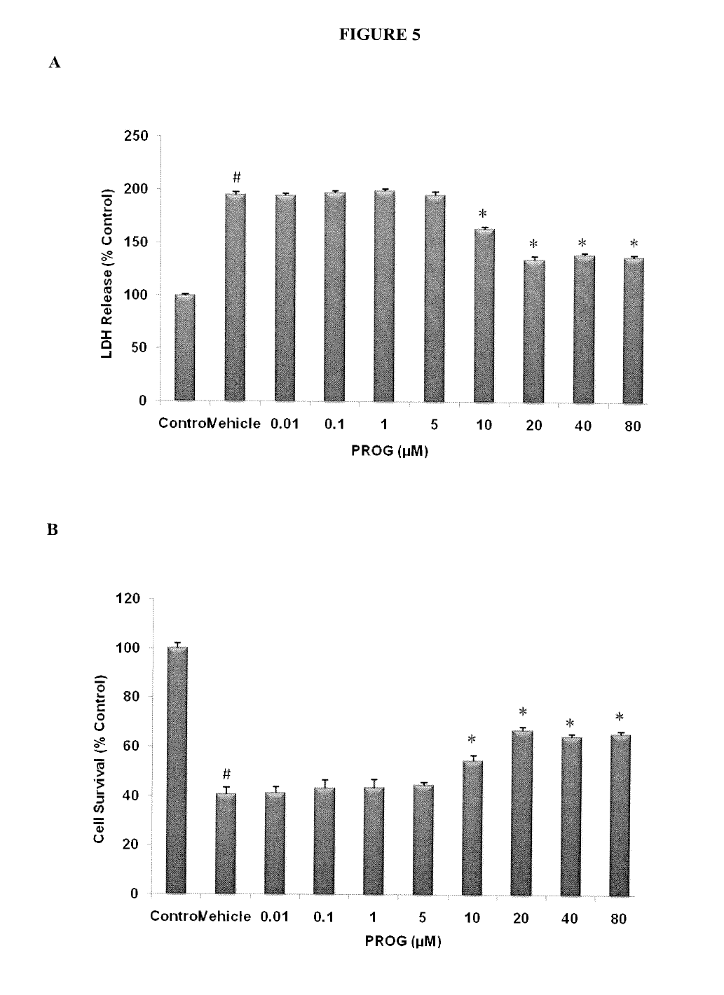

Glutamate exposure (0.5 μM for 24 h) resulted in a significant (P<0.001) increase in cell death in primary cortical neurons as compared to control cells exposed to solvent. The concentration-response curve for PROG against glutamate-induced cell death revealed that PROG at lower concentrations (0.01, 0.1, 1, 5 μM) did not show any decrease in cell death compared to the vehicle-only group. At higher concentrations (10, 20, 40, and 80 μM), a significant reduction (P<0.001) in cell death was observed as measured by both LDH and MTT assays (FIGS. 5A, 5B). The best concentration of PROG (alone) against glutamate-induced neuronal death was found to be 20 μM.

2. Neuroprotective Effect of VDH Against Glutamate-Induced Excitotoxic Cell Death

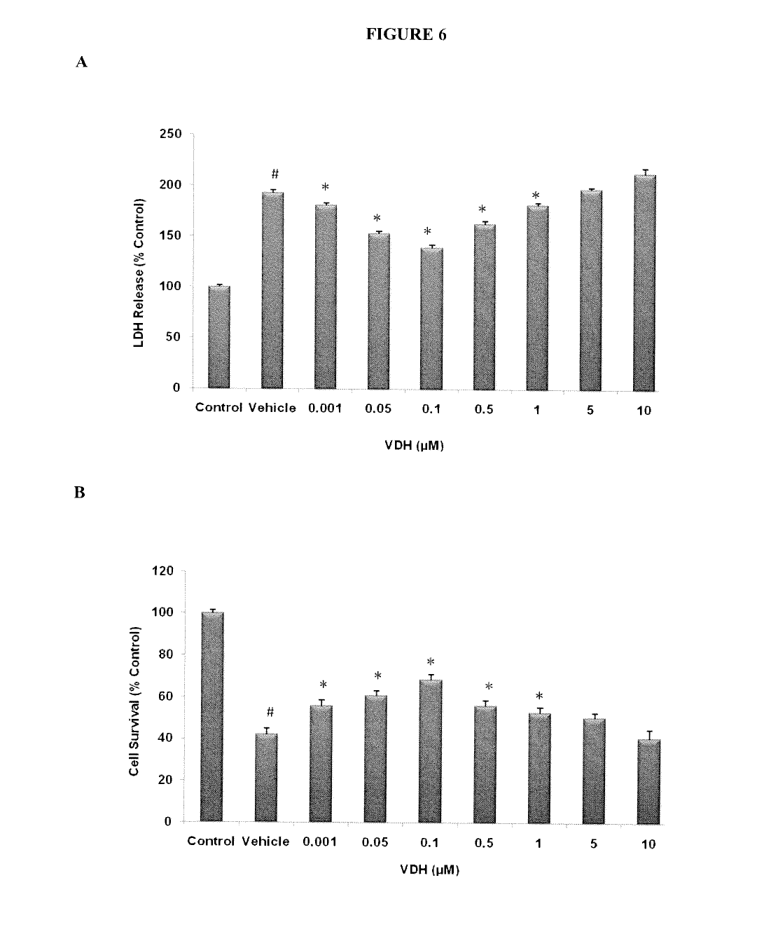

Different concentrations of VDH were tested against glutamate insult in primary cortical neurons. It was observed that VDH exhibited a “U-shaped” concentration response curve for neuroprotection against glutamate toxicity. Lower concentrations (0.001-0.5 μM) were significantly protective (P<0.001), while higher concentrations (1-10 μM) did not prevent neuronal loss compared to the vehicle-only control group. Both cell death assays suggested that VDH (alone) is most effective at 0.1 μM concentration (FIGS. 6A, 6B).

3. Combined Effect of PROG and VDH Against Glutamate-Induced Excitotoxic Cell Death

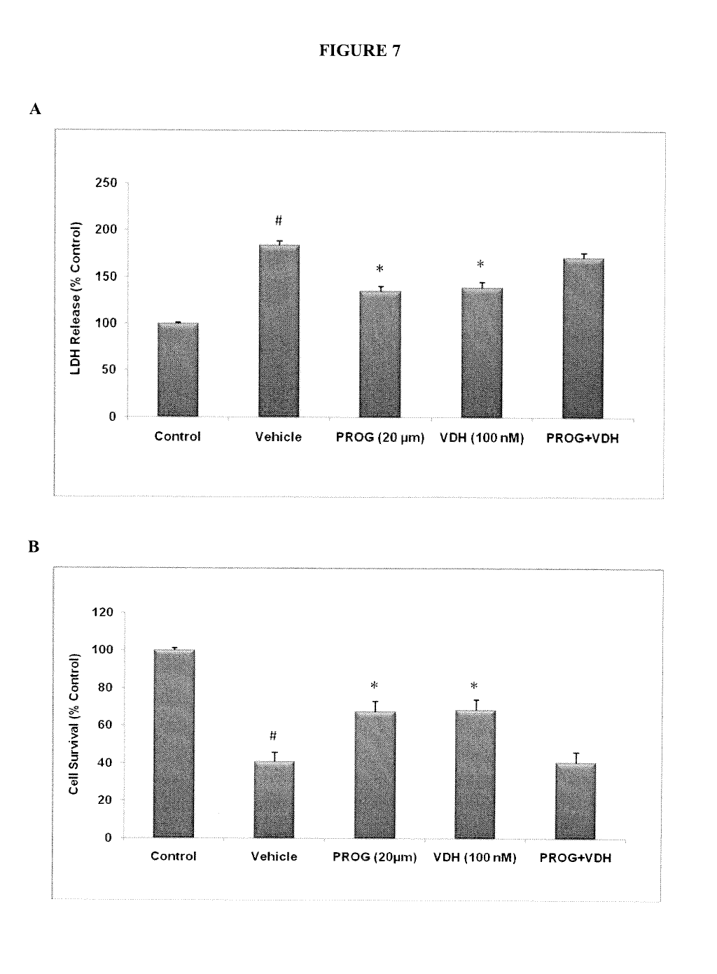

On the basis of the concentration-response curves obtained as described above, the most effective concentrations of PROG (20 μM) and VDH (0.1 μM) were combined and tested against glutamate toxicity in primary cortical neurons. The rationale behind combining only the most effective concentrations was that both the drugs were most neuroprotective individually at these concentrations (P<0.001) and therefore likely to show an additive or synergistic effect in combination at the same concentrations. It was found, however, that this combination treatment with PROG and VDH did not prevent cell death compared to vehicle (FIGS. 7A, 7B). In light of this finding and because VDH showed a U-shaped response curve against glutamate toxicity, it was deemed unlikely that higher concentrations of VDH would have a better outcome in combination with most effective concentration of PROG.

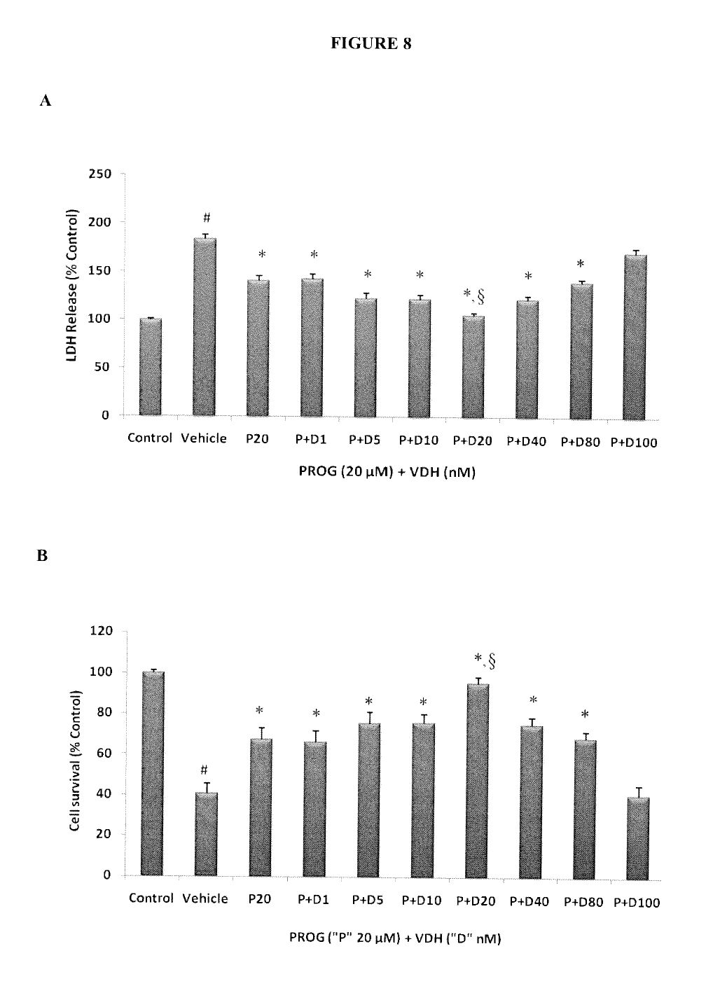

In antoher study, the most protective concentration of PROG (20 μM) was combined with different, lower concentrations of VDH (1, 5, 10, 20, 40, 80, and 100 nM) to evaluate the best combination of VDH with PROG. Both cell death assays showed that PROG and VDH given together produced a U-shaped concentration response curve for neuroprotection against glutamate-induced neuronal death (FIGS. 8A, 8B). The most effective combination was PROG (20 μM)+VDH (20 nM), which significantly reduced neuronal loss (P<0.001) compared to vehicle. Also, this combinatorial effect was significantly better (P<0.01) than the individual effect of either PROG (20 μM) or VDH (0.1 μM) at their most effective concentrations.

4. Morphological Changes In Primary Cortical Cultures After Drug Treatments

Changes in the morphology of neurons treated with the different agents and doses were observed. The control group showed neuritic processes characteristic of neurons in vitro. The glutamate (0.5 μM for 24 h) group showed the loss of neuronal processes and number of neurons. PROG and VDH alone rescued neurons independently but this neuroprotective effect was more pronounced when neurons were exposed to both PROG and VDH in combination. (Data not shown.)

5. Effect of PROG and VDH on MAPK Activation

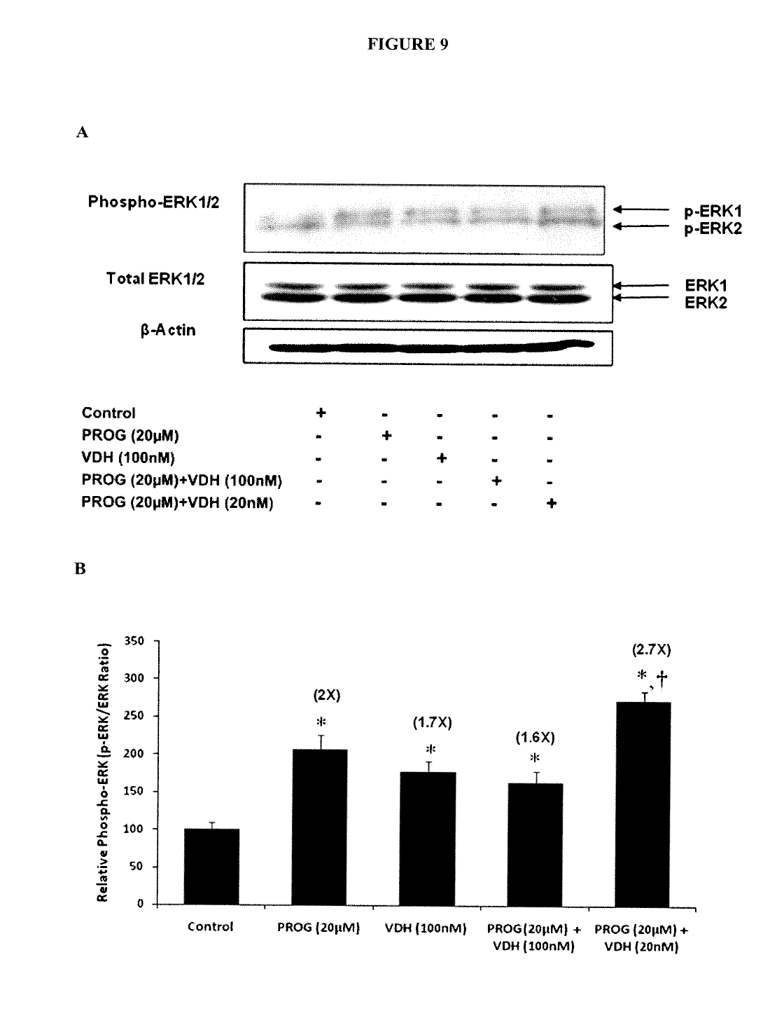

To examine the involvement of MAPK in the synergy of PROG- and VDH-mediated neuroprotection, MAPK phosphorylation was assessed in primary cortical neurons after 30-minute hormone treatment alone or in different combinations. Treatment with the most neuroprotective concentration of PROG (20 μM) and VDH (100 nM) resulted in a 2× and 1.7× increase, respectively, in p-ERK1/2 level compared to base-line phosphorylation values in the control group (FIG. 9B). The combination of these doses showed no additive effect and ERK1/2 phosphorylation was less (1.6×) than for PROG alone. The best combination dose of PROG and VDH (20 μM and 20 nM, respectively) seen in cell death assays (LDH and MTT) resulted in a 2.7× increase in MAPK activation.

### Example 10

### Possible Mechanisms of VDH and Progesterone Action

While not being bound by any theory, it is interesting to note that PROG and VDH affect many of the same as well as a number of divergent processes that are involved in the repair of secondary injury following TBI. Table 2 below summarizes some of the nuroprotective mechanisms of PROG and VDH. Identical mechanisms are identified by light grey shading, while divergent mechanisms are white. In case of a stronger response with reference to one mechanism, a double indicator is used (†\ versus †):

Key: †=increases, ←=decreases, >=greater than skew or bias, ∇=modulates.

TABLE 2
Neuroprotective mechanisms of PROG and VDH

| MECHANISM | PROGESTERONE | VDH |
|---|---|---|
| NEURONAL | ↓ cytochrome c | ↓ cytochrome c |
| APOPTOSIS | ↓ bad, bax, caspase-3 | ↓ cell cycle (neurons) |
| | ↑ bcl-2 | |
| | ∇ mitochondrial | ∇ n-myc, c-myc |
| | function | |
| TROPHIC | ↑ NGF, BDNF | ↑↑ NGF, BDNF |
| FACTORS | | ↑ GDNF, NT-4, TGFβ |
| | | ∇ IGFBPs |
| INFLAM- | ↓ GFAP | ↓ GFAP |
| MATION | ↓ TNFα, IL-1 | ↓ TNFα, IL-1 |
| | ↓ NFκB | ↓ NFκB |
| | T_{H}2 > T_{H}1 | T_{H}2 >> T_{H}1 |
| | | (↑ IL-4, ↓ IL-12, IFNγ) |
| | ↓ complement (C3, C5) | ∇ antigen-presenting |
| | | cells |
| | ∇ coagulation | ∇ immune proliferation |
| OXIDATIVE | ↓ lipid peroxidation | ↓ lipid peroxidation |
| STRESS | ↓ iNOS, NO, nitrites | ↓ iNOS, NO, nitrites |
| | ↓ immune ROS | ↓ immune ROS |
| | ↓ toxicity | ↓ toxicity |
| | (Fe, MPTP, β-amyloid) | (Fe, Zn, 6-OH dopamine) |
| | ↑ glutathione | ↑ glutathione |
| | ↓ MnSOD | ↓ HO-1 |
| | ↑ SOD | ↑ γ-GT |
| EXCITO- | ↑ GABA_{A} | ↓ L-VSCCs |
| TOXICITY/Ca²⁺ | ∇ σ1 receptor | ↑ Ca²⁺ buffering |
| | | (calbindin, parvalbumin) |
| MYELIN/ | ∇ MBP | ↑ axogenesis |
| AXONS | ↑ myelination | ↑ axon diameter |
| | (oligodendrocytes/CNS) | |
| | ↑ rcmyelination | |
| | (Schwann cells/PNS) | |
| OTHER | ∇ AQP4 | ∇ Renin-angiotensin |
| | ∇ Pgp (BBB function) | |
| | ∇ ChAT (NBM) | |
| | ∇ Na⁺, K⁺-ATPase | |
| | ↓ Edema | |

Abbreviations used in Table 2 are as follows:

| | | | |
|---|---|---|---|
| bad | Bcl-2 associated | TH1 | t-helper cell type 1 |
| | death protein | TH2 | t-helper cell type 2 |
| bax | Bcl-2 associated X | IFN γ | interferon γ |
| | protein | iNOS | Inducible nitric oxide |
| bcl-2 | B-cell leukemia 2 | | synthase |
| NGF | nerve growth factor | NOS | nitric oxide synthase |
| BDNF | brain-derived | ROS | reactive oxygen species |
| | neurotrophic factor | MPTP | 1-methyl-4-phenyl- |
| GDNF | glial cell derived | | 1,2,3,6-tetrahydro- |
| | neurotrophic factor | | pyridine |
| NT-4 | neurotrophin 4 | SOD | superoxide dismuatase |
| TGFβ | transforming growth | HO-1 | hemc oxygenase-1 |
| | factor β | γ-GT | γ-glutamyl |
| IGFBP | insulin like growth | | trasnpeptidase |
| | factor binding protein | GABA | γ-aminobutyric acid |
| GFAP | glial fibrillary | L-VSCC | L-type voltage sensitive |
| | acidic protein | | calcium channels |
| TNFα | tumor necrosis factor α | | |
| IL | interleukin | | |
| NF-kB | nuclear factor kappa- | | |
| | light chain enhancer | | |
| | of activated B cells | | |

Mcchanisms by which VDH complements PROG activity include, but are not limited to, the following.

1. Diminishing the effects of glutamate release and calcium influx:

VDH maintains intracellular Ca2+ through downregulating L-VSCCs and upregulating intracellular Ca2+ buffering capacity.

2. Protecting against the toxic effects of heme breakdown products:

VDH has been reported to upregulate glial home oxygenase-1 (HO-1) concomitantly with a reduction in GFAP following focal cortical ischemia. HO-1 is one of the rapidly induced heat shock proteins which metabolizes and thus detoxifies free heme to the powerful endogenous antixodants biliverdin, CO and Fe2+. Studies suggest that HO-1 induction by VDH protects cells from the oxidative toxicity of free hcmc.

3. Enhancing free radical scavenging:

VDH induces the expression of γ-GT and significantly increases intracellular glutathione in response to LPS-induced oxidative stress in astrocytes and protects neurons from chemical toxicity.

4. Modulating the renin-angiotensin system:

VDH plays an important role in the regulation of renin biosynthesis and blood pressure homeostasis. It also functions as an endocrine suppressor of renin biosynthesis and genetic disruption of the VDR results in overstimulation of the renin-angiotensin system (RAS), leading to high blood pressure and cardiac hypertrophy.

5. Protecting the axonal and cytoskeleton infrastructure:

VDH potentiates axon regeneration in a rat model of peripheral nerve injury. Following nerve injury, treatment with vitamin D2 (100 IU/kg/day) significantly increased axogenesis and axon diameter, improved the response of sensory neurons to metabolites such as KCl and lactic acid, and induced a fast-to-slow fiber type transmission of the Tibialis anterior muscle.

Thus, VDH not only shares many CNS repair mechanisms with PROG, but also contributes mechanisms of action that compensate for missing mechanisms in PROG's arsenal.

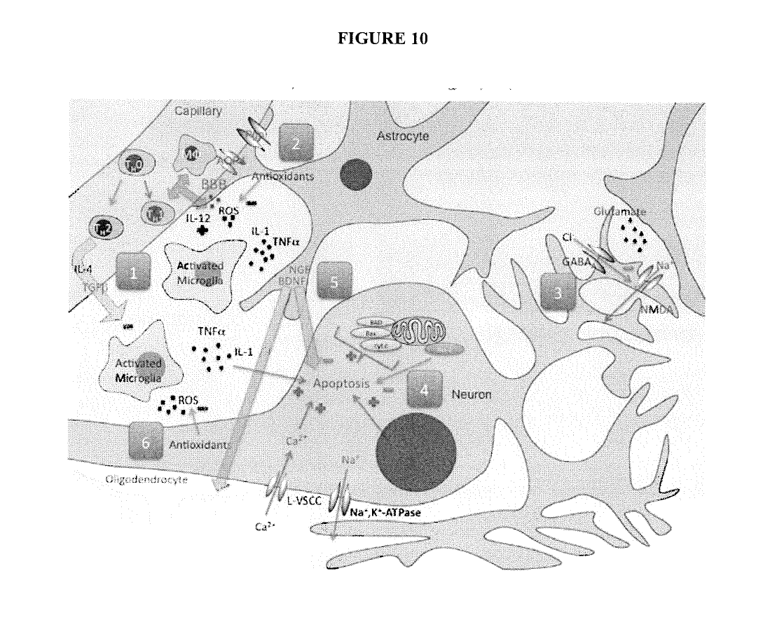

As a further illustration, brain injury processes affected by PROG and VDH are shown in FIG. 10. Both PROG and VDH are plieotropic and affect multiple pathways, which may account for their therapeutic effectiveness. A few of the major pathways involved in injury are shown in FIG. 10:

(1) Inflammatory pathways consisting of immune cell recruitment and infiltration (macrophages; McD), microglial activation and inflammatory cytokine release (TNFα and IL-1), and naive T cell (T_{H}0) differentiation into pro-inflammatory type 1 (T_{H}1) and anti-inflammatory type 2 (T_{H}2). These processes can lead to cell death, edema, and secondary damage.

(2) Maintenance of blood-brain barrier (BBB) integrity, including modulation of the expression of channels and transporters such as P-glycoprotein (Pgp) and aquaporin-4 (AQP4) and antioxidant protection for both capillary endothelium and astrocytes. Failure of BBB function is a key component in the development of edema.

(3) Glutamate excitotoxicity, mediated primarily by NMDA channels, can be toxic to the cell due to Na⁺ influx and severe depolarization. These effects can be counteracted by C1⁻ influx through GABA_{A} channels, leading to repolarization.

(4) The balance of cellular pro- and anti-death mechanisms, including release of pro-apoptotic mitochondrial (Bax, BAD, cytochrome c) and anti-apoptotic (Bcl-2) proteins, caspase-3 activation, maintenance of ionic and energy balance, as well as reduction of Ca²⁺ influx, which is the final common pathway of most mechanisms of cell death including glutamate toxicity. Since the activation of cellular reproductive machinery in terminally differentiated neurons can also lead to apoptosis, arrest of the cell cycle can also be protective.

(5) Upregulation of trophic factors, especially NGF and BDNF, which contribute not only to the maintenance of neurons and astrocytes, but also oligodendrocytes and myelination.

(6) Antioxidant defenses, which reduce the damage of immune and endogenously released reactive oxygen species (ROS) to cellular components and membranes. L-VSCC: L-type voltage-sensitive Ca²⁺ channel: Na⁺, K⁺-ATPase: Na⁺/K⁺ active transport pump.

Insults to the CNS, including TBI, induce neuroinflammatory and oxidative stress reactions, which then induce the secondary cascade of brain damage. As noted above, both FROG and VDH are pleiotropic hormones acting on several common, as well as on independent, CNS pathway mechanisms to reduce CNS damage and enhance CNS repair after TBI. As shown above, given the wide spectrum of action by the two hormones, the combination of the two, operating through unique and slightly different but compatible molecular mechanisms, are synergistic in reducing the cytotoxic events associated with the injury cascade and increasing the neuroprotective events related to anti-apoptotic signaling and brain repair.

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only, with the true scope and spirit of the invention being indicated by the following claims.

1. A pharmaceutical composition comprising:
(a) a neuroprotective steroid or a pharmaceutically acceptable salt, ester or prodrug thereof; and
(b) vitamin D,
optionally in a pharmaceutically acceptable carrier.

2. The composition of claim 1, wherein said neuroprotective steroid is selected from the group consisting of progesterone and allopregnanolone.

3. The composition of claim 1, wherein said neuroprotective steroid is represented by formula (I): 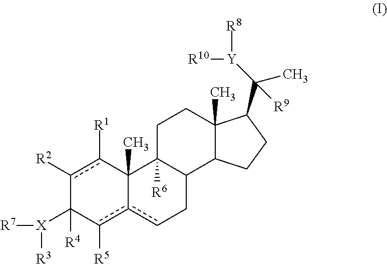
wherein X is O, N or S;
Y is O, N or S;
R¹, R², R⁵ and R⁶ are independently hydrogen, alkyl, halogen, hydroxylcycloalkyl, cycloalkenyl, alkenyl, alkynyl, aryl, alkylaryl, arylalkyl, heterocyclic, heteroaryl, amino, thiol, alkoxy, sulfide, nitro, cyano, azide, sulfonyl, acyl, carboxyl, an ester, an amide, carbamate, carbonate, an amino acid residue or a carbohydrate;
R⁴ is hydrogen or alkyl; or R⁴ and R⁷ together form a double bond;
R³ is hydrogen, optionally substituted acyl, a residue of an amino acid, a carbohydrate, —OR¹¹, —NR¹¹R¹² or R³ is absent;
R⁷ is hydrogen or is absent, or R⁷ together with R⁴ forms a double bond;
R⁸ is hydrogen, optionally substituted acyl, a residue of an amino acid, a carbohydrate, —OR¹¹, —NR¹¹R¹² or R⁸ absent;
R⁹ is hydrogen or alkyl; or R⁹ and R¹⁰ together form a double bond;
R¹⁰ is hydrogen or is absent, or R¹⁰ together with R⁹ forms a double bond;
R¹¹ is the residue of an amino acid, a carbohydrate or an optionally substituted ester or a substituted acyl;
R¹² is hydrogen or alkyl; and
the dotted line indicates the presence of either a single bond or a double bond, wherein the valences of a single bond are completed by hydrogens,
provided that
at least one of XR³R⁷ or YR⁸R¹⁰ is not ═O or OH, and that if the dotted line between C4 and C5 or between C5 and C6 represents a double bond then the other dotted line between C4 and C5 or between C5 and C6 represents a single bond; and with the proviso that neither XR³R⁷ nor YR⁸R¹⁰ represent an ester of aspartic acid, glutamic acid, gama amino butyric acid or a-2-(hydroxyethylamino)-propionic acid; and
with the proviso that when Y is N, R⁸ does not represent aspartic acid, glutamic acid, gama amino butyric acid or a-2-(hydroxyethylamino)-propionic acid.

4. The composition of claim 1, wherein said Vitamin D is selected from the group consisting of ergocalciferol, cholecalciferol, calcitriol, seocalcitol, doxercalciferol and calcipotriene.

5. The composition of claim 1, wherein said Vitamin D comprises a 1,25-dihydroxyvitamin D₃ (1,25-diOH-D).

6. The composition of claim 1, comprising an amount of vitamin D selected from the group consisting of (i) at least 1000 international units (IU), (ii) at least 1500 IU, (iii) at least 2000 IU, (iv) at least 2500 IU, (v) at least 3000 IU, (vi) at least 3500 IU, (vii) at least 4000 IU, (viii) at least 5000 IU, (ix) at least 10,000 IU, (x) at least 25,000 IU, and (xi) at least 50,000 IU.

7. The composition of claim 1, comprising an amount of neuroprotective steroid or a pharmaceutically acceptable salt, ester or prodrug thereof selected from the group consisting of (i) 0.1 mg to 5000 mg, (ii) 0.5 mg to 1000 mg, and (iii) 1 mg to 500 mg.

8. The composition of claim 1, formulated for oral, nasal, intravenous, or intramuscular administration.

9. Use of a composition as claimed in claim 1, for treating or preventing nervous system injury in a patient in need thereof.

10. A method of treating or preventing nervous system injury in a patient in need thereof, comprising administering to said patient:
(i) a neuroprotective steroid or a pharmaceutically acceptable salt, ester or prodrug thereof, and
(ii) vitamin D.

11. The method of claim 10, wherein said neuroprotective steroid is selected from the group consisting of progesterone and allopregnanolone.

12. The method of claim 10, wherein said vitamin D is selected from the group consisting of ergocalciferol, cholecalciferol, calcitriol, seocalcitol, doxercalciferol and calcipotriene.

13. The method of claim 10, wherein said vitamin D comprises a 1,25-dihydroxyvitamin D₃ (1,25-diOH-D).

14. The method of claim 10, wherein said neuroprotective steroid and vitamin D are administered in the same composition or in different compositions.

15. The method of claim 10, wherein said vitamin D is administered in an amount selected from the group consisting of (i) at least 1000 international units (IU), (ii) at least 1500 IU, (iii) at least or at least 2000 IU, (iv) at least 2500 IU, (v) at least 3000 IU, (vi) at least 3500 IU, and (vii) least 4000 IU, (viii) at least 5000 IU, (ix) at least 10,000 IU, (x) at least 25,000 IU, and (xi) at least 50,000 IU.

16. The method of claim 10, wherein the neuroprotective steroid or pharmaceutically acceptable salt, ester or prodrug thereof is administered in an amount selected from the group consisting of (i) 0.1 mg to 5000 mg, (ii) 0.5 mg to 1000 mg, and (iii) 1 mg to 500 mg.

17. The method claim 10, wherein the neuroprotective steroid or pharmaceutically acceptable salt, ester or prodrug thereof is administered in an amount selected from the group consisting of (i) 0.001 mg/kg/day to 1000 mg/kg/day, (ii) 0.05 mg/kg/day to 500 mg/kg/day, and (iii) 0.1 mg/kg/day to 300 mg/kg/day.

18. The method of claim 10, wherein the neuroprotective steroid or pharmaceutically acceptable salt, ester or prodrug thereof is administered orally, nasally, intravaneously, or intramuscularly.

19. The method of claim 10, wherein said method is commenced at a time selected from the group consisting of (i) one day from the nervous system injury; (ii) less than one day from the nervous system injury; (iii) less than 18 hours from the nervous system injury; (iv) less than 12 hours from the nervous system injury; and (v) less than six hours from the nervous system injury.

20.-30. (canceled)
